(19) United States
(12) Patent Application Publication
(10) Pub. No.: US 2021/0115520 A1
(43) Pub. Date: Apr. 22, 2021

(54) SYSTEMS AND METHODS FOR USING PATHOGEN NUCLEIC ACID LOAD TO DETERMINE WHETHER A SUBJECT HAS A CANCER CONDITION

(71) Applicant: GRAIL, Inc., Menlo Park, CA (US)

(72) Inventors: M. Cyrus Maher, San Mateo, CA (US); Anton Valouev, Palo Alto, CA (US); Seyedmehdi Shojaee, San Francisco, CA (US); Oliver Claude Venn, San Francisco, CA (US)

(21) Appl. No.: 17/050,372

(22) PCT Filed: Apr. 24, 2019
(86) PCT No.: PCT/US19/28916

§ 371 Date: Oct. 23, 2020

Related U.S. Application Data
(60) Provisional application No. 62/662,198, filed on Apr. 24, 2018.

(51) Int. Cl. C12Q 1/6886; G16B 40/20; C12Q 1/70; G16B 20/20; G16B 20/10; G16B 30/10

(52) U.S. Cl. CPC C12Q 1/6886; G16B 40/20; C12Q 1/701; G16B 20/20; C12Q 2600/154; G16B 30/10; C12Q 2600/158; C12Q 2600/156; G16B 20/10

(57) ABSTRACT

Methods for screening for a cancer condition in a subject are provided. A biological sample from the subject is obtained. The sample comprises cell-free nucleic acid from the subject and potentially cell-free nucleic acid from a pathogen in a set of pathogens. The cell-free nucleic acid in the biological sample is sequenced to generate a plurality of sequence reads from the subject. A determination is made, for each respective pathogen in the set of pathogens, of a corresponding amount of the plurality of sequence reads that map to a sequence in a pathogen target reference for the respective pathogen, thereby obtaining a set of amounts of sequence reads, each respective amount of sequence reads in the set of amounts of sequence reads for a corresponding pathogen in the set of pathogens. The set of amounts of sequence reads is used to determine whether the subject has the cancer condition.

### CROSS REFERENCE TO RELATED APPLICATION

This application is related to U.S. Provisional Patent Application No. 62/662,198 entitled “Systems and Methods for Using Pathogen Nucleic Acid Load to Determine Whether a Subject Has a Cancer Condition,” filed Apr. 24, 2018, which is hereby incorporated by reference.

### TECHNICAL FIELD

This specification describes using cell free nucleic acid obtained from a subject to classify a disease state or condition of the subject.

### BACKGROUND

It is estimated that approximately one in five cancers worldwide is linked to an infectious agent. See, de Flora, 2011, Carcinogenesis 32:787-795. Oncogenic viruses include hepatitis virus B and C (HBV and HCV), human papillomavirus (HPV), Epstein-Barr virus (EBV), human T-cell lymphoma virus 1 (HTLV-1), Merkel cell polyomavirus (MCPyV), and Kaposi's sarcoma virus also known as human herpes virus 8 (KSVH or HHV8)]. Oncogenic bacterium includes Helicobacter pylori. Oncogenic parasites include Schistosoma haematobium, Opithorchis viverrini, and Clonorchis sinensis. See, Vandeven, 2014, Cancer Immunol. Res. 2(1):9-14, and FIGS. 3A and 3B, reproduced from Vandeven.

Viruses can cause cellular transformation by expression of viral oncogenes, by genomic integration to alter the activity of cellular proto-oncogenes or tumor suppressors, and by inducing inflammation that promotes oncogenesis. See, Tang,” et al., 2013, Nature Communications 4:2513. For instance, as illustrated in FIG. 4 reproduced from Tang, Tang discloses RNA-seq-derived expression levels for 28 viruses (vertical axis) detected at 42 p.p.m. of total library reads in at least one tumor, across 178 virus-positive tumors from 19 cancer types (horizontal axis). In Tang, as summarized in FIG. 9 reproduced from Tang, non-human reads were matched to a database of 3,590 RefSeq viral genomes, that was complemented with 12 additional known and 2 partial novel genomes detected by de novo assembly of viral reads. Tang identified 178 tumors with FVR (viral expression) 42 p.p.m., but found that most positive cases had considerably higher levels (on average 168 and up to 854 p.p.m.).

Viral load is particularly evident in cervical carcinoma (CESC), which is almost exclusively caused by high-risk human papillomaviruses (HPV), and in hepatocellular carcinoma (LIHC), where infection with hepatitis B virus (HBV) or hepatitis C virus (HCV) is the predominant cause in some countries. See, Williams, 2006, Hepatology 44, 521-526. Additionally, cancers having a strong viral component include Epstein-Barr virus (EBV)/human herpes virus (HHV) 4 in most Burkitt's lymphomas. Advances in the prevention of virus-associated cancer has been made through vaccination programs against HPV and HBV, second only to smoke cessation in the number of yearly cancer cases prevented worldwide. See, Strong et al., 2008, Eur. J. Cancer Prev. 17, 153-161.

Cells infected with virus typically respond with an innate immune response that often includes releasing cytokines, which have been linked to oxidative stress, and stimulation of pro-growth transduction factors. Cytokines are known to trigger AID/APOBEC expression. It is known that the resulting AID/APOBEC proteins can cause hypermutation within the infected cells. Therefore, AID/APOBEC expression serves as a potential link between viral infection and malignant transformation. See, Siriwardena et al., 2016, Chem Rev, 116(20): 12688-12710. There are several reports linking APOBEC proteins to virus-driven tumor development, in particular, HPV and HBV: expression of APOBEC and mutational signatures occurs with high frequency in HPV-positive cervical and head-and-neck cancer (see Alexandrov et al., 2013, Nature, 500(7463), 415-421), and HBV driven hepatocellular carcinoma (see Deng et al., 2014, Cancer Lett. 343(2):161-71).

Virus-tumor associations to date have been determined by low-throughput methodologies in the pre-genomic era. However, massively parallel sequencing, including next generation sequencing, is now showing promise for efficient unbiased detection of viruses in tumor tissue. Such sequencing efforts led to the discovery of a new polyomavirus as the cause of most Merkel cell carcinomas. See, Feng et al., 2008, Science 319, 1096-1100. As an additional example, techniques for detection of viruses using high-throughput RNA or DNA sequencing are disclosed in Isakov et al., 2011, Bioinformatics 27, 2027-2030 and Kostic et al., 2012, Genome Res. 22, 292-298). As another example, massively parallel sequencing has been used to survey sites of genomic integration of HBV in hepatocellular carcinoma. See, Sung et al., 2012, Nat. Genet. 44, 765-769, and Jiang et al., 2012, Genome Res. 22, 593-601. Similarly, viral integration sites have been mapped in a number of cervical and head and neck carcinomas by detecting host-virus fusions in transcriptome sequencing (RNA-seq) data from The Cancer Genome Atlas (TCGA). See, Chen et al., 2013, Bioinformatics 29, 266-267. These studies provide important insights and clearly demonstrate the potential of using massively parallel sequencing to detect association between viruses and cancer conditions. However, such efforts are just beginning, and better assays and diagnostic algorithms are needed to make better use of the potential wealth of information regarding viruses and their association with cancer.

Given the above background, robust techniques for using information regarding viral load in subjects to identify a cancer condition in subjects are needed in the art.

### SUMMARY

The present disclosure addresses the shortcomings identified in the background by providing robust techniques for using information regarding viral load in subjects to identify a cancer condition in subjects are needed in the art.

I. Detection of pathogen load by itself (e.g., using targeted panel sequencing, whole genome sequencing, or whole genome bisulfite sequencing). One aspect of the present disclosure provides a method of screening for a cancer condition in a test subject based on genetic material that is derived from one or more pathogens. As disclosed herein, a pathogen can be a virus, a bacterium, a parasite, or any organism that is external to the test subject organism. As disclosed herein, a virus or a viral load is often used to illustrate the concepts. However, such illustration should not limit the scope in any way. The method comprises obtaining a first biological sample from the test subject. The first biological sample comprises cell-free nucleic acid from the test subject and potentially cell-free nucleic acid from at least one pathogen in a set of pathogens. In the method, the cell-free nucleic acid in the first biological sample is sequenced (e.g., by whole genome sequencing, targeted panel sequencing: methylation or non-methylation related, or whole genome bisulfite sequencing, etc.) to generate a plurality of sequence reads from the test subject. Further in the method, for each respective pathogen in the set of pathogens, a corresponding amount of the plurality of sequence reads that map to a sequence in a pathogen target reference for the respective pathogen is determined, thereby obtaining a set of amounts of sequence reads. Each respective amount of sequence reads in the set of amounts of sequence reads is for a corresponding pathogen in the set of pathogens. In the methods, the set of amounts of sequence reads is used to determine whether the test subject has the cancer condition or a likelihood that the test subject has the cancer condition.

In some embodiments, the method further comprises evaluating the plurality of sequence reads to obtain an indication as to whether an APOBEC induced mutational signature associated with a first pathogen in the set of pathogens is present or absent. In such embodiments, the indication as to whether the APOBEC induced mutational signature associated with the first pathogen is present or absent along with the set of amounts of sequence reads is used to determine whether the test subject has the cancer condition or the likelihood that the test subject has the cancer condition.

In some embodiments, the method further comprises evaluating, via k-mer analysis, the plurality of sequence reads to obtain an indication as to whether an APOBEC induced mutational signature is present or absent. In such embodiments, the indication as to whether the APOBEC induced mutational signature associated with the first pathogen is present or absent along with the set of amounts of sequence reads is used to determine whether the test subject has the cancer condition or the likelihood that test subject has the cancer condition.

In some embodiments, the method further comprises analyzing the first or second biological sample from the test subject for an expression of an APOBEC protein associated with a first pathogen in the set of pathogens. In such embodiments, the expression of the APOBEC protein and the set of amounts of sequence reads is used to determine whether the test subject has the cancer condition or the likelihood that the test subject has the cancer condition.

In some embodiments, the method relies upon a targeted gene panel that includes genetic markers corresponding to target sequences from various pathogens. For instance, in some such embodiments, the pathogen target reference for the respective pathogen consists of a targeted panel of sequences from the reference genome for the respective pathogen and the determining step limits, for a respective pathogen, the mapping of each sequence read in the plurality of sequence reads to the corresponding targeted panel of sequences from the reference genome of the respective pathogen.

In one aspect, an amount reflecting a viral load is compared to a reference/cutoff value. For example, values are computed for each subject in a training set to construct standard specificity and sensitivity curves (e.g., where the x-axis represents values of viral loads). The reference/cutoff value is chosen based on a desired target specificity. Alternatively, the overall viral loads or pathogen-based individual viral loads can be used directly as input to a classifier (e.g., a logistic regression based classifier). In some embodiments, the using set of amounts of sequence reads to determine whether the test subject has the cancer condition or a likelihood that the test subject has the cancer condition comprises determining a reference amount of sequence reads for a first pathogen in the set of pathogens associated with a predetermined percentile of a first distribution. In such embodiments, each respective subject in a first cohort of subjects contributes to the first distribution an amount of sequence reads from the respective subject that map to a sequence in the pathogen target reference for the first pathogen. Each subject in a first portion of the first cohort of subjects has the cancer condition, and each subject in a second portion of the first cohort of subjects does not have the cancer condition. Then, what is compared is (i) a first amount that is the amount of the plurality of sequence reads that map to a sequence in the pathogen target reference for the first pathogen from the test subject and (ii) a second amount that is the reference amount of sequence reads for the first pathogen in the set of pathogens associated with the predetermined percentile of the first distribution. When the first amount exceeds the second amount (a reference/cutoff value is chosen based on a desired target specificity) by a threshold amount the likelihood that the test subject has the cancer condition is specified or a determination is made that the test subject has the cancer condition.

As disclosed herein, an amount (e.g., the first or second amount) can be a value reflecting an abundance level of nucleic acid fragments in the cell-free nucleic acid sample that are derived from a pathogen. For example, an amount here can be a concentration, a ratio of viral-derived sequence reads over sequence reads derived from the test subject (e.g., a human), or any suitable measure where the viral-derived sequence reads are evaluated within a context.

In one aspect, a normalized pathogen load is compared to a reference/cutoff value. For example, a training set and a control healthy set are used. The training set includes both healthy and diseased subjects. In some embodiments, the control healthy set can be a subset of the training set. In some embodiments, pathogen loads are normalized by a certain percentile in pathogen loads of healthy samples in the healthy set to render a normalized viral load for each pathogen type. In some embodiments, the normalized loads are then summed to provide an overall pathogen load. The training set is used to construct specificity and sensitivity curves (e.g., where the x-axis represents values of overall pathogen load or a normalized load for a given pathogen). A reference/cutoff value is chosen based on a desired target specificity. Alternatively, the overall viral loads or pathogen-based individual viral loads can be used directly as input to a classifier (e.g., a logistic regression based classifier). In some such embodiments, the using the set of amounts of sequence reads to determine whether the test subject has the cancer condition or a likelihood that the test subject has the cancer condition comprises determining a reference amount of sequence reads for a first pathogen in the set of pathogens associated with a predetermined percentile of a first distribution (e.g., 90%, 95%, 98%, or another suitable percentage). In some such embodiments, the using the set of amounts of sequence reads to determine whether the test subject has the cancer condition or a likelihood that the test subject has the cancer condition comprises determining a reference amount of sequence reads for a first pathogen in the set of pathogens associated with a predetermined percentile of a first distribution. Each respective subject in a first cohort of subjects that do not have the cancer condition contributes to the first distribution an amount of sequence reads from the respective subject that map to a sequence in the pathogen target reference for the first pathogen.

In one aspect, instead of using cut off values, the ratios from each subject in the training set or the normalized pathogen load values from each subject in the training set are used as input in a binomial or multinomial classification algorithm. In some such embodiments, the using the set of amounts of sequence reads to determine whether the test subject has the cancer condition or a likelihood that the test subject has the cancer condition comprises applying the set of amounts of sequence reads to a classifier to thereby determine either (i) whether the test subject has the cancer condition or (ii) the likelihood that test subject has the cancer condition.

In some embodiments, the determining step comprises thresholding the corresponding amount of the plurality of sequence reads that map to a sequence in the pathogen target reference for the respective pathogen based on an amount of sequence reads associated with a predetermined percentile of a respective distribution. Each respective subject in a respective cohort of subjects that do not have the cancer condition contributes to the respective distribution an amount of sequence reads from the respective subject that map to a sequence in the pathogen target reference for the respective pathogen, thereby determining a scaled respective amount of the plurality of sequence reads from the test subject. In such embodiments, the test subject is determined to have the cancer condition or the likelihood that the test subject has the cancer condition when a classifier inputted with at least each scaled respective amount of the plurality of sequence reads from the test subject indicates that the test subject has the cancer condition. In some such embodiments, the classifier is based on a logistic regression algorithm that individually weights each scaled respective amount of the plurality of sequence reads based on a corresponding amount of sequence reads mapping to a sequence in the pathogen target reference of the corresponding pathogen observed in a training cohort of subjects that includes subjects that have the cancer condition and subjects that do not have the cancer condition. In some such embodiments, the set of pathogens comprises between 2 and 100 pathogens.

II. Detection of a pathogen load in conjunction with another type of analysis (e.g., copy number aberration analysis by whole genome sequencing or methylation analysis by whole genome bisulfate sequencing). Another aspect of the present disclosure provides a method of screening for a cancer condition in a test subject. The method comprises obtaining a first biological sample from the test subject that comprises test-free nucleic acid from the test subject and potentially cell-free nucleic acid from at least one pathogen in a set of pathogens. The method further comprises performing a first assay comprising measuring an amount of a first feature of the cell-free nucleic acid in the first biological sample. The method further comprises performing a second assay comprising i) sequencing the cell-free nucleic acid in a second biological sample to generate a plurality of sequence reads from the test subject, where the second biological sample is from the test subject, and where the second biological sample comprises cell-free nucleic acid from the test subject and potentially cell-free nucleic acid from at least one pathogen in the set of pathogens, and ii) determining, for each respective pathogen in the set of pathogens, a corresponding amount of the plurality of sequence reads that map to a sequence in a pathogen target reference for the respective pathogen, thereby obtaining a set of amounts of sequence reads, each respective amount of sequence reads in the set of amounts of sequence reads for a corresponding pathogen in the set of pathogens. The method further comprises screening for the cancer condition based on the first and second assay, where the test subject is deemed to have a likelihood of having the cancer condition or to have the cancer condition when either the first assay or the second assay, or both the first assay and the second assay, indicate that the test subject has or does not have the cancer condition or provides a likelihood that the test subject has or does not have the cancer condition.

In some embodiments, the method further comprises evaluating the plurality of sequence reads to obtain an indication as to whether an APOBEC induced mutational signature associated with a first pathogen in the set of pathogens is present or absent. In such embodiments, the screening uses (i) the indication as to whether the signature fragment signature associated with a first pathogen is present or absent, (ii) the amount of the first feature, and (iii) the indication as to whether the APOBEC induced mutational signature associated with the first pathogen is present or absent to determine whether the test subject has the cancer condition or the likelihood that the test subject has the cancer condition.

In some embodiments, the screening uses (i) the indication as to whether the signature fragment signature associated with a first pathogen is present or absent, (ii) the amount of the first feature, and (iii) the indication as to whether the APOBEC induced mutational signature associated with the first pathogen is present or absent to determine whether the test subject has the cancer condition or the likelihood that the test subject has the cancer condition.

In some embodiments, the indication as to whether the APOBEC induced mutational signature associated with the first pathogen is present or absent further includes a measure of enrichment of the APOBEC induced mutational signature. In such embodiments, the screening uses (i) the indication as to whether the signature fragment signature associated with a first pathogen is present or absent, (ii) the amount of the first feature, and (iii) the measure of enrichment of the APOBEC induced mutational signature to determine whether the test subject has the cancer condition or the likelihood that the test subject has the cancer condition.

In one aspect, the second assay comprises determining an amount reflecting a viral load by comparing it to a reference/cutoff value. For example, values are computed for each subject in a training set to construct standard specificity and sensitivity curves (e.g., where the x-axis represents values of viral loads). The reference/cutoff value is chosen based on a desired target specificity. Alternatively, the overall viral loads or pathogen-based individual viral loads can be used directly as input to a classifier (e.g., a logistic regression based classifier). In some embodiments, the second assay further comprises determining a reference amount of sequence reads for a first pathogen in the set of pathogens associated with a predetermined percentile of a first distribution. Each respective subject in a first cohort of subjects contributes to the first distribution an amount of sequence reads from the respective subject that map to a sequence in the pathogen target reference for the first pathogen. Each subject in a first portion of the first cohort of subjects has the cancer condition and each subject in a second portion of the first cohort of subjects does not have the cancer condition. A first amount that is the amount of the plurality of sequence reads that map to a sequence in a pathogen target reference for the first pathogen from the test subject is compared to a second amount that is the reference amount of sequence reads for the first pathogen in the set of pathogens associated with the predetermined percentile of the first distribution. When the first amount exceeds the second amount by a threshold amount the second assay dictates a likelihood that the test subject has the cancer condition or determines that the test subject has the cancer condition.

In one aspect, the second assay comprises determining a normalized pathogen load, which is then compared to a reference/cutoff value. For example, a training set and a control healthy set are used. The training set includes both healthy and diseased subjects. In some embodiments, the control healthy set can be a subset of the training set. In some embodiments, pathogen loads are normalized by a certain percentile in pathogen loads of healthy samples in the healthy set to render a normalized pathogen load for each pathogen type. In some embodiments, the normalized loads are then summed to provide an overall pathogen load. The training set is used to construct specificity and sensitivity curves (e.g., where the x-axis represents values of overall pathogen load or a normalized load for a given pathogen). A reference/cutoff value is chosen based on a desired target specificity. Alternatively, the overall pathogen loads or pathogen-based individual pathogen loads are used directly as input to a classifier (e.g., a logistic regression based classifier). In some embodiments, a reference amount of sequence reads for a first pathogen in the set of pathogens associated with a predetermined percentile of a first distribution (e.g., 90%, 95%, 98%, or another suitable percentage) is determined. Each respective subject in a first cohort of subjects that do not have the cancer condition contributes to the first distribution an amount of sequence reads from the respective subject that map to a sequence in the pathogen target reference for the first pathogen. The amount of the plurality of sequence reads that map to a sequence in a pathogen target reference for the first pathogen from the test subject is thresholded by the reference amount of sequence reads for the first pathogen in the set of pathogens associated with the predetermined percentile of the first distribution to thereby form a scaled amount of the plurality of sequence reads. The scaled amount of the plurality of sequence reads is compared to a scaled amount of the plurality of sequence reads associated with a predetermined percentile of a second distribution. Each respective subject in a second cohort of subjects contributes to the second distribution a scaled amount of sequence reads from the respective subject that map to a sequence in the pathogen target reference for the first pathogen. Each subject in a first portion of the subjects in the second cohort have the cancer condition and each subject in a second portion of the subjects in the second cohort do not have the cancer condition.

In one aspect, in the second assay, instead of using cutoff values, the ratios from each subject in the training set or the normalized pathogen load values from each subject in the training set can be used as input in a binomial or multi-normal classification algorithm. In some embodiments the performing the second assay further comprises applying the corresponding amount of sequence reads that map to a sequence in the pathogen target reference for the respective pathogen to a classifier to thereby have the second assay call either (i) whether the test subject has the cancer condition or (ii) a likelihood that test subject has the cancer condition.

In one aspect, the second assay comprises pathogen load analysis performed in combination with the present of a test subject derived signature for cancer detection (e.g., a signature for copy number aberration analysis, a signature for somatic mutation analysis, or a signature for methylation analysis). In one aspect, pathogen load analysis is performed in combination with the presence of a pathogen specific signature, and further in combination with the presence of a test subject derived signature for cancer detection (e.g., a signature for copy number aberration analysis, a signature for somatic mutation analysis, or a signature for methylation analysis). In some embodiments, the method further comprises evaluating the plurality of sequence reads to obtain an indication as to whether a sequence fragment signature associated with a first pathogen in the set of pathogens is present or absent. The method further comprises evaluating the plurality of sequence reads to obtain an indication as to whether a methylation signature associated with the first pathogen in the set of pathogens is present or absent. In such embodiments, the screening for the cancer condition uses (i) the indication as to whether the signature fragment signature associated with the first pathogen is present or absent, (ii) an indication as to whether a methylation signature associated with the first pathogen is present or absent, (iii) the amount of the first feature, and (iv) the set of amounts of sequence reads to determine whether the test subject has the cancer condition or the likelihood that test subject has the cancer condition.

In some embodiments, the performing the second assay further comprises, for each respective pathogen in the set of pathogens, thresholding the corresponding amount of the plurality of sequence reads that map to a sequence in the pathogen target reference for the respective pathogen on an amount of sequence reads associated with a predetermined percentile of a respective distribution. In such embodiments, each respective subject in a respective cohort of subjects that do not have the cancer condition contributes to the respective distribution an amount of sequence reads from the respective subject that map to a sequence in the pathogen target reference for the respective pathogen, thereby determining a scaled respective amount of the plurality of sequence reads from the test subject. In such embodiments, the test subject is deemed by the second assay to have the likelihood of having the cancer condition or to have the cancer condition when a classifier inputted with at least each scaled respective amount of the plurality of sequence reads from the test subject indicates that the test subject has the cancer condition.

In some embodiments, the classifier is a logistic regression that individually weights each scaled respective amount of the plurality of sequence reads based on a corresponding amount of sequence reads mapping a sequence in the pathogen target reference for the respective pathogen observed in a training cohort of subjects that includes subjects that have the cancer condition and subjects that do not have the cancer condition.

In some embodiments, the performing the second assay further comprises, for each respective pathogen in the set of pathogens, thresholding the corresponding amount of the plurality of sequence reads that map to a sequence in the pathogen target reference for the respective pathogen on an amount of sequence reads associated with a predetermined percentile of a respective distribution, where each respective subject in a respective cohort of subjects that do not have the cancer condition contributes to the respective distribution an amount of sequence reads from the respective subject that map to a sequence in the pathogen target reference for the respective pathogen, thereby determining a scaled respective amount of the plurality of sequence reads from the test subject. In such embodiments, each scaled respective amount of the plurality of sequence reads from the test subject is summed to determine an overall oncopathogen load. The second assay indicates that the test subject has the cancer condition when the overall oncopathogen load satisfies a threshold cutoff condition.

In some embodiments, the threshold cutoff condition is a predetermined specificity for overall oncopathogen load across the set of pathogens determined for a pool of subjects that do not have the cancer condition. In some embodiments, the predetermined specificity is the 95^{th} percentile.

In some embodiments, the first assay has a sensitivity for a first set of markers indicative of the cancer condition, and the first feature is one of a copy number, a fragment size distribution, a fragmentation pattern, a methylation status, or a mutational status of the cell-free nucleic acid in the first biological sample across the first set of markers.

In some embodiments, the amount of the first feature is thresholded on an amount of the first feature associated with a predetermined percentile of a second distribution to thereby form a scaled amount of the first feature. Each respective subject in a second cohort of subjects that do not have the cancer condition contributes to the second distribution a value for the first feature measured from the respective subject. The test subject is deemed by the first assay to have the cancer condition when the scaled amount of the first feature exceeds the amount of the first feature associated with the predetermined percentile of the second distribution by a second predetermined cutoff value.

In some embodiments the method further comprises providing a therapeutic intervention or imaging of the test subject based on an outcome of the screening for the cancer condition based upon the above disclosed combination of the first assay and the second assay.

III. The presence of viral specific signatures for detection of a cancer condition. Another aspect of the present disclosure provides a method of screening for a cancer condition in a test subject. A first biological sample, comprising cell-free nucleic acid from the test subject and potentially cell-free nucleic acid from at least one pathogen in a set of pathogens, is obtained from the test subject. The cell-free nucleic acid is sequenced to generate a plurality of sequence reads The sequence reads are evaluated to obtain an indication as to whether a sequence fragment signature associated with a respective pathogen in the set of pathogens is present or absent. The indication as to whether the signature fragment signature associated with the respective pathogen is present or absent is used to determine whether the test subject has the cancer condition or the likelihood that test subject has the cancer condition. In some embodiments, the method further comprises evaluating the plurality of sequence reads to obtain an indication as to whether an APOBEC induced mutational signature associated with a first pathogen in the set of pathogens is present or absent. In such embodiments, the indication as to whether the APOBEC induced mutational signature associated with the first pathogen is present or absent along with the indication as to whether the signature fragment signature associated with the respective pathogen is present or absent is used to determine whether the test subject has the cancer condition or the likelihood that the test subject has the cancer condition.

In some embodiments, the indication as to whether the APOBEC induced mutational signature associated with the first pathogen is present or absent along with the indication as to whether the signature fragment signature associated with the respective pathogen is present or absent is used to determine whether the test subject has the cancer condition or the likelihood that the test subject has the cancer condition.

In some embodiments, the measure of enrichment of the APOBEC induced mutational signature along with the indication as to whether the signature fragment signature associated with the respective pathogen is present or absent is used to determine whether the test subject has the cancer condition or the likelihood that the test subject has the cancer condition.

In some embodiments, the expression of the APOBEC protein along with an indication as to whether the signature fragment signature associated with the respective pathogen is present or absent is used to determine whether the test subject has the cancer condition or the likelihood that the test subject has the cancer condition.

In some embodiments, the method further comprises performing an assay comprising measuring an amount of an APOBEC induced mutational signature of the cell-free nucleic acid in the first biological sample. In such embodiments, the amount of the APOBEC induced mutational signature and the set of amounts of sequence reads is used to determine whether the test subject has the cancer condition or the likelihood that the test subject has the cancer condition.

IV. The presence of a methylation signature for detection of a cancer condition. Another aspect of the present disclosure provides a method of screening for a cancer condition in a test subject in which a first biological sample is obtained from the test subject. The first biological sample comprises cell-free nucleic acid from the test subject and potentially cell-free nucleic acid from at least one pathogen in a set of pathogens. The cell-free nucleic acid is sequenced to generate a plurality of sequence reads that are evaluated to obtain an indication as to whether a methylation signature associated with a respective pathogen in the set of pathogens is present or absent. The indication as to whether the methylation signature associated with the respective pathogen is present or absent is used to determine whether the test subject has the cancer condition or the likelihood that test subject has the cancer condition.

V. The presence of a pathogen specific signature and a methylation signature for detection of a cancer condition. Another aspect of the present disclosure provides a method of screening for a cancer condition in a test subject in which a first biological sample is obtained from the test subject. The first biological sample comprises cell-free nucleic acid from the test subject and potentially cell-free nucleic acid from at least one pathogen in a set of pathogens. The cell-free nucleic acid is sequenced to generate a plurality of sequence reads that are evaluated to obtain an indication as to whether a sequence fragment signature associated with a respective pathogen in the set of pathogens is present or absent. The plurality of sequence reads are further evaluated to obtain an indication as to whether a methylation signature associated with a respective pathogen in the set of pathogens is present or absent. The indication as to whether the signature fragment signature associated with a respective pathogen is present or absent and the indication as to whether the methylation signature associated with a respective pathogen is present or absent are used to determine whether the test subject has the cancer condition or the likelihood that test subject has the cancer condition.

In some embodiments, the indication as to whether the APOBEC induced mutational signature associated with the first pathogen is present or absent along with the indication as to whether the methylation signature associated with the respective pathogen is present or absent are used to determine whether the test subject has the cancer condition or the likelihood that the test subject has the cancer condition.

In some embodiments, the indication as to whether the APOBEC induced mutational signature associated with the first pathogen is present or absent along with the indication as to whether the methylation signature associated with the respective pathogen is present or absent are used to determine whether the test subject has the cancer condition or the likelihood that test subject has the cancer condition.

In some embodiments, the measure of enrichment of the APOBEC induced mutational signature along with the indication as to whether the methylation signature associated with the respective pathogen is present or absent are used to determine whether the test subject has the cancer condition or the likelihood that the test subject has the cancer condition.

In some embodiments, the expression of the APOBEC protein along with the indication as to whether the methylation signature associated with the respective pathogen is present or absent are used to determine whether the test subject has the cancer condition or the likelihood that the test subject has the cancer condition.

In some embodiments, the indication as to whether the APOBEC induced mutational signature associated with the first pathogen is present or absent along with the indication as to whether the methylation signature associated with the respective pathogen is present or absent are used to determine whether the test subject has the cancer condition or the likelihood that the test subject has the cancer condition.

In some embodiments, the method proceeds by performing an assay comprising measuring an amount of an APOBEC induced mutational signature of the cell-free nucleic acid in the second biological sample. In such embodiments, the indication as to whether the APOBEC induced mutational signature associated with the first pathogen is present or absent along with the indication as to whether the methylation signature associated with the respective pathogen is present or absent are used to determine whether the test subject has the cancer condition or the likelihood that the test subject has the cancer condition. In some such embodiments, the sequencing is performed by whole genome sequencing, targeted panel sequencing (methylation or non-methylation related), or whole genome bisulfite sequencing.

VI. Pathogen-derived panel for cancer screening. Another aspect of the present disclosure provides a pathogen panel for screening for a test subject to determine a likelihood or indication that the subject has a cancer condition, the viral panel comprising a first and second sequence fragment. In some embodiments, the first sequence fragment encodes at least 100 bases of the genome of the corresponding parasite. In some embodiments, the pathogen panel includes a sequence fragment for at least 4, at least 5, at least 8, or at least 50 different parasites in the set of parasites. In some embodiments, the first sequence fragment encodes a portion of a protein encoded by the genome of the corresponding parasite. In some embodiments, the first sequence fragment encodes a methylation pattern of a portion of the genome of the corresponding parasite.

VII. Methods for screening for a cancer condition based on the presence of cell-free nucleic acid from one or more pathogens. Another aspect of the present disclosure provides a method of screening for a cancer condition in a test subject. The method comprises obtaining a first biological sample from the test subject. The first biological sample comprises cell-free nucleic acid from the test subject and potentially cell-free nucleic acid from a first pathogen in a set of pathogens. The method further comprises performing an assay in which cell-free nucleic acid in the first biological sample are sequenced to generate a plurality of sequence reads from the test subject. The assay further comprises determining an amount of the plurality of sequence reads that align to a reference genome of the first pathogen. The assay further comprises thresholding the amount on an amount of sequence reads associated with a predetermined percentile of a first distribution. Each respective subject in a cohort of subjects that do not have the cancer condition contributes to the first distribution an amount of sequence reads from the respective subject that align to the reference genome of the first pathogen, thereby determining a scaled first amount of the plurality of sequence reads from the test subject. The test subject is deemed to have the cancer condition when a metric based, at least in part, on the scaled first amount of the plurality of sequence reads satisfies a threshold associated with the cancer condition.

In some embodiments, the test subject is deemed to have the cancer condition when a metric, based on the APOBEC induced mutational signature associated with the first pathogen is present or absent and the scaled first amount of the plurality of sequence reads, satisfies a threshold associated with the cancer condition.

In some embodiments, the test subject is deemed to have the cancer condition when a metric, based on the APOBEC induced mutational signature associated with the first pathogen is present or absent and the scaled first amount of the plurality of sequence reads, satisfies a threshold associated with the cancer condition. In some embodiments, the test subject is deemed to have the cancer condition when a metric, based on the measure of enrichment of the APOBEC induced mutational signature and the scaled first amount of the plurality of sequence reads, satisfies a threshold associated with the cancer condition. In some embodiments, the test subject is deemed to have the cancer condition when a metric, based on the expression of an APOBEC protein associated with a first pathogen in the set of pathogens and the scaled first amount of the plurality of sequence reads, satisfies a threshold associated with the cancer condition. In some embodiments, the test subject is deemed to have the cancer condition when a metric, based on the amount of an APOBEC induced mutational signature and the scaled first amount of the plurality of sequence reads, satisfies a threshold associated with the cancer condition. In some embodiments, the test subject is deemed to have the cancer condition when a metric, based on the amount of an APOBEC induced mutational signature and the scaled first amount of the plurality of sequence reads, satisfies a threshold associated with the cancer condition.

In some embodiments, the test subject is deemed by the assay to have the cancer condition when the scaled first amount of the plurality of sequence reads from the test subject exceeds the amount of sequence reads associated with the predetermined percentile of the distribution by a predetermined cutoff value. In some embodiments, the first predetermined cutoff value is a single standard deviation greater than a measure of central tendency of the distribution. In some embodiments, the first predetermined cutoff value is three standard deviations greater than a measure of central tendency of the distribution.

VIII. Methods for screening for multiple cancer conditions based on presence of cell-free nucleic acid from one or more pathogens. Another aspect of the present disclosure provides a method of screening for each cancer condition in a plurality of cancer conditions in a test subject in which a first biological sample is obtained from the test subject. The first biological sample comprises cell-free nucleic acid from the test subject and potentially cell-free nucleic acid from any pathogen in a set of pathogens. The cell-free nucleic acid in the first biological sample is sequenced to generate a plurality of sequence reads from the test subject. The method further comprises performing a procedure, for each respective pathogen in the set of pathogens. The procedure comprises determining a respective amount of the plurality of sequence reads that align to a reference genome of the respective pathogen, and thresholding the respective amount on an amount of sequence reads associated with a predetermined percentile of a respective distribution. Each respective subject in a respective cohort of subjects that do not have a cancer condition in the plurality of cancer conditions contributes to the respective distribution an amount of sequence reads from the respective subject that align to the reference genome of the respective pathogen, thereby determining a scaled respective amount of the plurality of sequence reads from the respective subject. The method further comprises inputting at least each scaled respective amount of the plurality of sequence reads into a classifier thereby obtaining a classifier result that indicates whether the test has a cancer condition in the plurality of cancer conditions.

In some embodiments, the indication as to whether the APOBEC induced mutational signature associated with the first pathogen is present or absent along with each scaled respective amount of the plurality of sequence reads are inputted into the classifier, thereby obtaining a classifier result that indicates whether the test has a cancer condition in the plurality of cancer conditions. In some embodiments, the indication as to whether the APOBEC induced mutational signature associated with the first pathogen is present or absent along with each scaled respective amount of the plurality of sequence reads is inputted into the classifier, thereby obtaining a classifier result that indicates whether the test has a cancer condition in the plurality of cancer conditions. In some embodiments, the measure of enrichment of the APOBEC induced mutational signature along with each scaled respective amount of the plurality of sequence reads are inputted into the classifier, thereby obtaining a classifier result that indicates whether the test has a cancer condition in the plurality of cancer conditions. In some embodiments, the method further comprises analyzing the first biological sample or a second biological sample from the test subject for an expression of an APOBEC protein associated with a first pathogen in the set of pathogens. In such embodiments, the expression of the APOBEC protein along with each scaled respective amount of the plurality of sequence reads are inputted into the classifier, thereby obtaining a classifier result that indicates whether the test has a cancer condition in the plurality of cancer conditions. In some embodiments, the amount of an APOBEC induced mutational signature along with each scaled respective amount of the plurality of sequence reads are inputted into the classifier, thereby obtaining a classifier result that indicates whether the test has a cancer condition in the plurality of cancer conditions.

In some embodiments, the method further comprises obtaining a second biological sample from the test subject, where the second biological sample comprises cell-free nucleic acid from the test subject and potentially cell-free nucleic acid from a first pathogen in the set of pathogens. In such embodiments, the amount of an APOBEC induced mutational signature along with each scaled respective amount of the plurality of sequence reads are inputted into the classifier, thereby obtaining a classifier result that indicates whether the test has a cancer condition in the plurality of cancer conditions.

In some embodiments, the set of pathogens comprises at least two pathogens. In some embodiments, the set of pathogens comprises at least twenty pathogens.

IX. Methods for screening for multiple cancer conditions based on presence of cell-free nucleic acid from one or more pathogens using a plurality of binomial classifiers. Another aspect of the present disclosure provides a method of screening for each cancer condition in a plurality of cancer conditions in a test subject. The method comprises obtaining a first biological sample from the test subject, where the first biological sample comprises cell-free nucleic acid from the test subject and potentially cell-free nucleic acid from any pathogen in a set of pathogens. The method further comprises sequencing of the cell-free nucleic acid in the first biological sample to generate a plurality of sequence reads from the test subject. The method further comprises performing a procedure, for each respective pathogen in the set of pathogens. The procedure comprises determining a respective amount of the plurality of sequence reads that align to a reference genome of the respective pathogen, and thresholding the respective amount on an amount of sequence reads associated with a predetermined percentile of a respective distribution. Each respective subject in a respective cohort of subjects that do not have a cancer condition in the plurality of cancer conditions contributes to the respective distribution an amount of sequence reads from the respective subject that align to the reference genome of the respective pathogen, thereby determining a scaled respective amount of the plurality of sequence reads from the respective subject. The method further comprises inputting at least each scaled respective amount of the plurality of sequence reads into each classifier in a plurality of classifiers, where each classifier in the plurality of classifier indicates whether the respective subject has or does not have a corresponding single cancer condition in the plurality of cancer conditions.

In some embodiments, the inputting step inputs the indication as to whether the APOBEC induced mutational signature associated with the first pathogen is present or absent along with each scaled respective amount of the plurality of sequence reads into each classifier in the plurality of classifiers. Each classifier in the plurality of classifier indicates whether the respective subject has or does not have a corresponding single cancer condition in the plurality of cancer conditions.

In some embodiments, the inputting step inputs the indication as to whether the APOBEC induced mutational signature associated with the first pathogen is present or absent along with each scaled respective amount of the plurality of sequence reads into each classifier in the plurality of classifiers. Each classifier in the plurality of classifier indicates whether the respective subject has or does not have a corresponding single cancer condition in the plurality of cancer conditions.

In some embodiments, the measure of enrichment of the APOBEC induced mutational signature along with each scaled respective amount of the plurality of sequence reads are inputted into each classifier in a plurality of classifiers. Each classifier in the plurality of classifier indicates whether the respective subject has or does not have a corresponding single cancer condition in the plurality of cancer conditions.

In some embodiments, the inputting step inputs the expression of the APOBEC protein along with each scaled respective amount of the plurality of sequence reads into each classifier in the plurality of classifiers. Each classifier in the plurality of classifier indicates whether the respective subject has or does not have a corresponding single cancer condition in the plurality of cancer conditions.

In some embodiments, the inputting step inputs the amount of an APOBEC induced mutational signature along with each scaled respective amount of the plurality of sequence reads into each classifier in the plurality of classifiers. Each classifier in the plurality of classifier indicates whether the respective subject has or does not have a corresponding single cancer condition in the plurality of cancer conditions.

In some embodiments, the inputting step inputs the amount of an APOBEC induced mutational signature along with each scaled respective amount of the plurality of sequence reads into each classifier in the plurality of classifiers. Each classifier in the plurality of classifier indicates whether the respective subject has or does not have a corresponding single cancer condition in the plurality of cancer conditions.

Other embodiments are directed to systems, portable consumer devices, and computer readable media associated with methods described herein. As disclosed herein, any embodiment disclosed herein when applicable can be applied to any aspect. Additional aspects and advantages of the present disclosure will become readily apparent to those skilled in this art from the following detailed description, where only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications herein are incorporated by reference in their entireties. In the event of a conflict between a term herein and a term in an incorporated reference, the term herein controls.

### BRIEF DESCRIPTION OF THE DRAWINGS

The implementations disclosed herein are illustrated by way of example, and not by way of limitation, in the figures of the accompanying drawings. Like reference numerals refer to corresponding parts throughout the several views of the drawings.

FIG. 1 illustrates an example block diagram illustrating a computing device in accordance with some embodiments of the present disclosure.

FIGS. 2A, 2B, 2C, 2D, 2E, 2F, 2G, 2H, 2I, 2J, 2K, 2L, and 2M collectively illustrate an example flowchart of a method of screening for a cancer condition in a test subject in accordance with some embodiments of the present disclosure.

FIGS. 3A and 3B illustrate the association of various cancers with pathogens such as viruses (e.g., hepatitis virus B and C (HBV and HCV), human papillomavirus (HPV), Epstein-Barr virus (EBV), human T-cell lymphoma virus 1 (HTLV-1), Merkel cell polyomavirus (MCPyV), and Kaposi's sarcoma virus), oncogenic bacterium including Helicobacter pylori, and oncogenic parasites including Schistosoma haematobium, Opithorchis viverrini, and Clonorchis sinensis, as disclosed in Vandeven, 2014, Cancer Immunol. Res. 2(1):9-14.

FIG. 4 illustrates the RNA-seq-derived expression levels for 28 viruses detected in 178 tumors in which the (vertical axis) detected at 42 p.p.m of total library reads in at least one tumor, across 178 virus-positive tumors from 19 cancer types (horizontal axis) as disclosed in Tang, 2013, Nature Communications 4:2513.

FIG. 5 illustrates the proportion of cancer subjects with detectable sequence reads from a virus as a function of cancer type, as well as the proportion of non-cancer subjects with detectable sequence reads from a virus in accordance with an embodiment of the present disclosure.

FIG. 6 illustrates the proportion of cancer subjects with detectable sequence reads by viral species further by cancer type in accordance with an embodiment of the present disclosure.

FIG. 7 illustrates the number of head and neck cancer cases detected using a viral load assay and a SCNA Z-score assay in accordance with an embodiment of the present disclosure.

FIG. 8 illustrates the number of cancer cases detected using a viral load assay and a SCNA Z-score assay (sensitivity) for various cancers in their early stages and late stage by thresholding against a cohort at 95 percent specificity in accordance with an embodiment of the present disclosure.

FIG. 9 illustrates bar graphs that show the fraction of tumors with strong viral expression (410 p.p.m. viral reads in library) as well as weaker detections (2-10 p.p.m.) and pie charts that show the relative numbers of positive tumors for major virus categories, with strong and weak detections shown separately as disclosed in in Tang, 2013, Nature Communications 4:2513.

FIG. 10 illustrates that among early-stage breast cancers uniquely identified by viral load, read counts using the disclosed techniques are well below the detection threshold of prior art studies.

FIG. 11 illustrates the number of cancer cases detected using a viral load assay and a SCNA Z-score assay (sensitivity) for various cancers in their early stages and late stage by thresholding against a cohort at 95 percent specificity in accordance with an embodiment of the present disclosure.

FIG. 12 illustrates, on a proportional basis, the representation of virus sequences, where the viruses where selected based upon their presence in top performing models for predicting cancer in accordance with an embodiment of the present disclosure.

FIG. 13 illustrates a distribution in which each respective subject in a first cohort of subjects contributes to the first distribution an amount of sequence reads from the respective subject that map to a sequence in the pathogen target reference for a first pathogen in accordance with an embodiment of the present disclosure.

FIG. 14 illustrates a distribution in which each respective subject in a cohort of subjects contributes to the distribution an amount of sequence reads from the respective subject that map to a sequence in the pathogen target reference for a first pathogen in accordance with an embodiment of the present disclosure.

FIG. 15 illustrates a second distribution in which each respective subject in a second cohort of subjects contributes to the second distribution an amount of sequence reads from the respective subject that map to a sequence in the pathogen target reference for a first pathogen in accordance with an embodiment of the present disclosure.

FIG. 16 illustrates a first distribution in which each respective subject in a second cohort of subjects contributes to the first distribution an amount of sequence reads from the respective subject that map to a sequence in the pathogen target reference for a first pathogen in accordance with an embodiment of the present disclosure.

FIG. 17 illustrates a first distribution in which each respective subject in a second cohort of subjects contributes to the second distribution an amount of sequence reads from the respective subject that map to a sequence in the pathogen target reference for a second pathogen in accordance with an embodiment of the present disclosure.

FIG. 18 is a flowchart of a method for obtaining a methylation information for the purposes of screening for a cancer condition in a test subject in accordance with some embodiments of the present disclosure.

FIG. 19 illustrates a flowchart of a method for preparing a nucleic acid sample for sequencing in accordance with some embodiments of the present disclosure.

FIG. 20 is a graphical representation of the process for obtaining sequence reads in accordance with some embodiments of the present disclosure.

### DETAILED DESCRIPTION

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

The implementations described herein provide various technical solutions for screening for a condition. A first assay quantifies an amount of a feature of cell-free nucleic acid in a first biological sample of a test subject. A second assay generate sequence reads from the cell-free nucleic acid in a second biological sample of the test subject. An amount of these sequence reads aligning to the pathogen reference genome is thresholded by an amount of sequence reads associated with a predetermined percentile of a distribution. Each respective subject in a cohort of subjects not having the condition contributes to the distribution an amount of sequence reads aligning to the pathogen reference genome. This results in a scaled amount of the sequence reads from the test subject. Screening for the condition is performed based on the first and second assays, making use of the scaled amount of the test subject sequence reads, in which the test subject is deemed to have the condition when either the first or second assay indicates the subject has the condition.

### Definitions

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within one or more than one standard deviation, per the practice in the art. “About” can mean a range of ±20%, ±10%, ±5%, or ±1% of a given value. The term “about” or “approximately” can mean within an order of magnitude, within 5-fold, or within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

As used herein, the term “assay” refers to a technique for determining a property of a substance, e.g., a nucleic acid, a protein, a cell, a tissue, or an organ. An assay (e.g., a first or second assay) can comprise a technique for determining the copy number variation of nucleic acids in a sample, the methylation status of nucleic acids in a sample, the fragment size distribution of nucleic acids in a sample, the mutational status of nucleic acids in a sample, or the fragmentation pattern of nucleic acids in a sample. Any assay known to a person having ordinary skill in the art can be used to detect any of the properties of nucleic acids mentioned herein. Properties of a nucleic acids can include a sequence, genomic identity, copy number, methylation state at one or more nucleotide positions, size of the nucleic acid, presence or absence of a mutation in the nucleic acid at one or more nucleotide positions, and pattern of fragmentation of a nucleic acid (e.g., the nucleotide position(s) at which a nucleic acid is fragmented). An assay or method can have a particular sensitivity and/or specificity, and their relative usefulness as a diagnostic tool can be measured using ROC-AUC statistics.

As used herein, the term “biological sample,” “patient sample,” or “sample” refers to any sample taken from a subject, which can reflect a biological state associated with the subject, and that includes cell free DNA. Examples of biological samples include, but are not limited to, blood, whole blood, plasma, serum, urine, cerebrospinal fluid, fecal, saliva, sweat, tears, pleural fluid, pericardial fluid, or peritoneal fluid of the subject. A biological sample can include any tissue or material derived from a living or dead subject. A biological sample can be a cell-free sample. A biological sample can comprise a nucleic acid (e.g., DNA or RNA) or a fragment thereof. The term “nucleic acid” can refer to deoxyribonucleic acid (DNA), ribonucleic acid (RNA) or any hybrid or fragment thereof. The nucleic acid in the sample can be a cell-free nucleic acid. A sample can be a liquid sample or a solid sample (e.g., a cell or tissue sample). A biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. A biological sample can be a stool sample. In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free). A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis.

As used herein the term “cancer” or “tumor” refers to an abnormal mass of tissue in which the growth of the mass surpasses and is not coordinated with the growth of normal tissue. A cancer or tumor can be defined as “benign” or “malignant” depending on the following characteristics: degree of cellular differentiation including morphology and functionality, rate of growth, local invasion, and metastasis. A “benign” tumor can be well differentiated, have characteristically slower growth than a malignant tumor and remain localized to the site of origin. In addition, in some cases a benign tumor does not have the capacity to infiltrate, invade, or metastasize to distant sites. A “malignant” tumor can be a poorly differentiated (anaplasia), have characteristically rapid growth accompanied by progressive infiltration, invasion, and destruction of the surrounding tissue. Furthermore, a malignant tumor can have the capacity to metastasize to distant sites.

The term “classification” can refer to any number(s) or other characters(s) that are associated with a particular property of a sample. For example, a “+” symbol (or the word “positive”) can signify that a sample is classified as having deletions or amplifications. In another example, the term “classification” can refer to an amount of tumor tissue in the subject and/or sample, a size of the tumor in the subject and/or sample, a stage of the tumor in the subject, a tumor load in the subject and/or sample, and presence of tumor metastasis in the subject. The classification can be binomial (e.g., positive or negative) or have more levels of classification (e.g., a scale from 1 to 10 or 0 to 1). The terms “cutoff” and “threshold” can refer to predetermined numbers used in an operation. For example, a cutoff size can refer to a size above which fragments are excluded. A threshold value can be a value above or below which a particular classification applies. Either of these terms can be used in either of these contexts.

As used herein, the terms “cell free nucleic acid(s),” “cell free DNA(s),” and “cfDNA(s)” interchangeably refer to nucleic acid fragments that circulate in a subject's bodily fluids (e.g., blood, whole blood, plasma, serum, urine, cerebrospinal fluid, fecal, saliva, sweat, sweat, tears, pleural fluid, pericardial fluid, or peritoneal fluid) and originate from one or more healthy cells and/or from one or more cancer cells. Cell-free nucleic acids are used interchangeably as circulating nucleic acids. Examples of the cell-free nucleic acids include but are not limited to RNA, mitochondrial DNA, or genomic DNA.

As used herein, the terms “control,” “control sample,” “reference,” “reference sample,” “normal,” and “normal sample” describe a sample from a subject that does not have a particular condition, or is otherwise healthy. In an example, a method as disclosed herein can be performed on a subject having a tumor, where the reference sample is a sample taken from a healthy tissue of the subject. A reference sample can be obtained from the subject, or from a database. The reference can be, e.g., a reference genome that is used to map sequence reads obtained from sequencing a sample from the subject. A reference genome can refer to a haploid or diploid genome to which sequence reads from the biological sample and a constitutional sample can be aligned and compared. An example of constitutional sample can be DNA of white blood cells obtained from the subject. For a haploid genome, there can be only one nucleotide at each locus. For a diploid genome, heterozygous loci can be identified; each heterozygous locus can have two alleles, where either allele can allow a match for alignment to the locus.

As used herein the term “ending position” or “end position” (or just “end”) can refer to the genomic coordinate or genomic identity or nucleotide identity of the outermost base, e.g., at the extremities, of a cell-free DNA molecule, e.g., plasma DNA molecule. The end position can correspond to either end of a DNA molecule. In this manner, if one refers to a start and end of a DNA molecule, both can correspond to an ending position. In some cases, one end position is the genomic coordinate or the nucleotide identity of the outermost base on one extremity of a cell-free DNA molecule that is detected or determined by an analytical method, e.g., massively parallel sequencing or next-generation sequencing, single molecule sequencing, double- or single-stranded DNA sequencing library preparation protocols, polymerase chain reaction (PCR), or microarray. In some cases, such in vitro techniques can alter the true in vivo physical end(s) of the cell-free DNA molecules. Thus, each detectable end can represent the biologically true end or the end is one or more nucleotides inwards or one or more nucleotides extended from the original end of the molecule e.g., 5′ blunting and 3′ filling of overhangs of non-blunt-ended double stranded DNA molecules by the Klenow fragment. The genomic identity or genomic coordinate of the end position can be derived from results of alignment of sequence reads to a human reference genome, e.g., hg19. It can be derived from a catalog of indices or codes that represent the original coordinates of the human genome. It can refer to a position or nucleotide identity on a cell-free DNA molecule that is read by but not limited to target-specific probes, mini-sequencing, DNA amplification. The term “genomic position” can refer to a nucleotide position in a polynucleotide (e.g., a gene, a plasmid, a nucleic acid fragment, a viral DNA fragment). The term “genomic position” is not limited to nucleotide positions within a genome (e.g., the haploid set of chromosomes in a gamete or microorganism, or in each cell of a multicellular organism).

As used herein, the term “false positive” (FP) refers to a subject that does not have a condition. False positive can refer to a subject that does not have a tumor, a cancer, a precancerous condition (e.g., a precancerous lesion), a localized, or a metastasized cancer, a non-malignant disease, or is otherwise healthy. The term false positive can refer to a subject that does not have a condition, but is identified as having the condition by an assay or method of the present disclosure.

As used herein, the term “fragment” (e.g., a DNA fragment), refers to a portion of a polynucleotide or polypeptide sequence that comprises at least three consecutive nucleotides. A nucleic acid fragment can retain the biological activity and/or some characteristics of the parent polynucleotide. In an example, nasopharyngeal cancer cells can deposit fragments of Epstein-Barr Virus (EBV) DNA into the bloodstream of a subject, e.g., a patient. These fragments can comprise one or more BamHI-W sequence fragments, which can be used to detect the level of tumor-derived DNA in the plasma. The BamHI-W sequence fragment corresponds to a sequence that can be recognized and/or digested using the Bam-HI restriction enzyme. The BamHI-W sequence can refer to the sequence 5′-GGATCC-3′.

As used herein, the term “false negative” (FN) refers to a subject that has a condition. False negative can refer to a subject that has a tumor, a cancer, a precancerous condition (e.g., a precancerous lesion), a localized or a metastasized cancer, or a non-malignant disease. The term false negative can refer to a subject that has a condition, but is identified as not having the condition by an assay or method of the present disclosure.

As used herein, the phrase “healthy,” refers to a subject possessing good health. A healthy subject can demonstrate an absence of any malignant or non-malignant disease. A “healthy individual” can have other diseases or conditions, unrelated to the condition being assayed, which can normally not be considered “healthy.”

As used herein, the term “informative cancer DNA fragment” or an “informative DNA fragment” can correspond to a DNA fragment bearing or carrying any one or more of the cancer-associated or cancer-specific change or mutation, or a particular ending-motif (e.g., a number of nucleotides at each end of the DNA fragment having a particular sequence).

As used herein, the term “level of cancer” refers to whether cancer exists (e.g., presence or absence), a stage of a cancer, a size of tumor, presence or absence of metastasis, the total tumor burden of the body, and/or other measure of a severity of a cancer (e.g., recurrence of cancer). The level of cancer can be a number or other indicia, such as symbols, alphabet letters, and colors. The level can be zero. The level of cancer can also include premalignant or precancerous conditions (states) associated with mutations or a number of mutations. The level of cancer can be used in various ways. For example, screening can check if cancer is present in someone who is not known previously to have cancer. Assessment can investigate someone who has been diagnosed with cancer to monitor the progress of cancer over time, study the effectiveness of therapies or to determine the prognosis. In one embodiment, the prognosis can be expressed as the chance of a subject dying of cancer, or the chance of the cancer progressing after a specific duration or time, or the chance of cancer metastasizing. Detection can comprise ‘screening’ or can comprise checking if someone, with suggestive features of cancer (e.g., symptoms or other positive tests), has cancer. A “level of pathology” can refer to level of pathology associated with a pathogen, where the level can be as described above for cancer. When the cancer is associated with a pathogen, a level of cancer can be a type of a level of pathology.

As used herein a “methylome” can be a measure of an amount of DNA methylation at a plurality of sites or loci in a genome. The methylome can correspond to all of a genome, a substantial part of a genome, or relatively small portion(s) of a genome. A “tumor methylome” can be a methylome of a tumor of a subject (e.g., a human). A tumor methylome can be determined using tumor tissue or cell-free tumor DNA in plasma. A tumor methylome can be one example of a methylome of interest. A methylome of interest can be a methylome of an organ that can contribute nucleic acid, e.g., DNA into a bodily fluid (e.g., a methylome of brain cells, a bone, lungs, heart, muscles, kidneys, etc.). The organ can be a transplanted organ.

As used herein the term “methylation index” for each genomic site (e.g., a CpG site) can refer to the proportion of sequence reads showing methylation at the site over the total number of reads covering that site. The “methylation density” of a region can be the number of reads at sites within a region showing methylation divided by the total number of reads covering the sites in the region. The sites can have specific characteristics, (e.g., the sites can be CpG sites). The “CpG methylation density” of a region can be the number of reads showing CpG methylation divided by the total number of reads covering CpG sites in the region (e.g., a particular CpG site, CpG sites within a CpG island, or a larger region). For example, the methylation density for each 100-kb bin in the human genome can be determined from the total number of unconverted cytosines (which can correspond to methylated cytosine) at CpG sites as a proportion of all CpG sites covered by sequence reads mapped to the 100-kb region. This analysis can also be performed for other bin sizes, e.g., 50-kb or 1-Mb, etc. A region can be an entire genome or a chromosome or part of a chromosome (e.g., a chromosomal arm). A methylation index of a CpG site can be the same as the methylation density for a region when the region only includes that CpG site. The “proportion of methylated cytosines” can refer the number of cytosine sites, “C's,” that are shown to be methylated (for example unconverted after bisulfite conversion) over the total number of analyzed cytosine residues, e.g., including cytosines outside of the CpG context, in the region. The methylation index, methylation density, and proportion of methylated cytosines are examples of “methylation levels.”

As used herein, the term “methylation profile” (also called methylation status) can include information related to DNA methylation for a region. Information related to DNA methylation can include a methylation index of a CpG site, a methylation density of CpG sites in a region, a distribution of CpG sites over a contiguous region, a pattern or level of methylation for each individual CpG site within a region that contains more than one CpG site, and non-CpG methylation. A methylation profile of a substantial part of the genome can be considered equivalent to the methylome. “DNA methylation” in mammalian genomes can refer to the addition of a methyl group to position 5 of the heterocyclic ring of cytosine (e.g., to produce 5-methyl cytosine) among CpG dinucleotides. Methylation of cytosine can occur in cytosines in other sequence contexts, for example 5′-CHG-3′ and 5′-CHH-3′, where H is adenine, cytosine, or thymine. Cytosine methylation can also be in the form of 5-hydroxymethylcytosine. Methylation of DNA can include methylation of non-cytosine nucleotides, such as N6-methyladenine.

As used herein, the term “mutation,” refers to a detectable change in the genetic material of one or more cells. In a particular example, one or more mutations can be found in, and can identify, cancer cells (e.g., driver and passenger mutations). A mutation can be transmitted from apparent cell to a daughter cell. A person having skill in the art will appreciate that a genetic mutation (e.g., a driver mutation) in a parent cell can induce additional, different mutations (e.g., passenger mutations) in a daughter cell. A mutation generally occurs in a nucleic acid. In a particular example, a mutation can be a detectable change in one or more deoxyribonucleic acids or fragments thereof. A mutation generally refers to nucleotides that is added, deleted, substituted for, inverted, or transposed to a new position in a nucleic acid. A mutation can be a spontaneous mutation or an experimentally induced mutation. A mutation in the sequence of a particular tissue is an example of a “tissue-specific allele.” For example, a tumor can have a mutation that results in an allele at a locus that does not occur in normal cells. Another example of a “tissue-specific allele” is a fetal-specific allele that occurs in the fetal tissue, but not the maternal tissue.

As used herein, the terms “nucleic acid” and “nucleic acid molecule” are used interchangeably. The terms refer to nucleic acids of any composition form, such as deoxyribonucleic acid (DNA, e.g., complementary DNA (cDNA), genomic DNA (gDNA) and the like), and/or DNA analogs (e.g., containing base analogs, sugar analogs and/or a non-native backbone and the like), all of which can be in single- or double-stranded form. Unless otherwise limited, a nucleic acid can comprise known analogs of natural nucleotides, some of which can function in a similar manner as naturally occurring nucleotides. A nucleic acid can be in any form useful for conducting processes herein (e.g., linear, circular, supercoiled, single-stranded, double-stranded and the like). A nucleic acid in some embodiments can be from a single chromosome or fragment thereof (e.g., a nucleic acid sample may be from one chromosome of a sample obtained from a diploid organism). In certain embodiments nucleic acids comprise nucleosomes, fragments, or parts of nucleosomes or nucleosome-like structures. Nucleic acids sometimes comprise protein (e.g., histones, DNA binding proteins, and the like). Nucleic acids analyzed by processes described herein sometimes are substantially isolated and are not substantially associated with protein or other molecules. Nucleic acids also include derivatives, variants and analogs of DNA synthesized, replicated or amplified from single-stranded (“sense” or “antisense,” “plus” strand or “minus” strand, “forward” reading frame or “reverse” reading frame) and double-stranded polynucleotides. Deoxyribonucleotides include deoxyadenosine, deoxycytidine, deoxyguanosine, and deoxythymidine. A nucleic acid may be prepared using a nucleic acid obtained from a subject as a template.

As used herein, a “pathogen” can be a virus, a bacterium, a parasite, or any organism that is external to the test subject organism. As disclosed herein, a virus or a viral load is often used to illustrate the concepts. However, such illustration should not limit the scope in any way.

As used herein, the term “reference genome” refers to any particular known, sequenced, or characterized genome, whether partial or complete, of any organism or virus that may be used to reference identified sequences from a subject. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species' set of genes. In some embodiments, a reference genome comprises sequences assigned to chromosomes. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38).

As used herein, the term “sequence reads” or “reads” refers to nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads). The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median, or average length of about 1000 bp, 2000 bp, 5000 bp, 10,000 bp, or 50,000 bp or more. Nanopore sequencing, for example, can provide sequence reads that can vary in size from tens to hundreds to thousands of base pairs. Illumina parallel sequencing can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

As used herein, the terms “sequencing,” “sequence determination,” and the like as used herein refers generally to any and all biochemical processes that may be used to determine the order of biological macromolecules such as nucleic acids or proteins. For example, sequencing data can include all or a portion of the nucleotide bases in a nucleic acid molecule such as a DNA fragment.

As used herein the term “sequencing depth” refers to the number of times a locus is covered by a sequence read aligned to the locus. The locus can be as small as a nucleotide, as large as a chromosome arm, or as large as an entire genome. Sequencing depth can be expressed as “Yx”, e.g., 50×, 100×, etc., where “Y” refers to the number of times a locus is covered with a sequence read. Sequencing depth can also be applied to multiple loci, or the whole genome, in which case Y can refer to the mean number of times a loci or a haploid genome, or a whole genome, respectively, is sequenced. When a mean depth is quoted, the actual depth for different loci included in the dataset can span over a range of values. Ultra-deep sequencing can refer to at least 100× in sequencing depth at a locus.

As used herein, the term “sensitivity” or “true positive rate” (TPR) refers to the number of true positives divided by the sum of the number of true positives and false negatives. Sensitivity can characterize the ability of an assay or method to correctly identify a proportion of the population that truly has a condition. For example, sensitivity can characterize the ability of a method to correctly identify the number of subjects within a population having cancer. In another example, sensitivity can characterize the ability of a method to correctly identify the one or more markers indicative of cancer.

As used herein, the term “single nucleotide variant” or “SNV” refers to a substitution of one nucleotide to a different nucleotide at a position (e.g., site) of a nucleotide sequence, e.g., a sequence read from an individual. A substitution from a first nucleobase X to a second nucleobase Y may be denoted as “X>Y.” For example, a cytosine to thymine SNV may be denoted as “C>T.”

As used herein, the terms “size profile” and “size distribution” can relate to the sizes of DNA fragments in a biological sample. A size profile can be a histogram that provides a distribution of an amount of DNA fragments at a variety of sizes. Various statistical parameters (also referred to as size parameters or just parameter) can distinguish one size profile to another. One parameter can be the percentage of DNA fragment of a particular size or range of sizes relative to all DNA fragments or relative to DNA fragments of another size or range.

As used herein, the term “specificity” or “true negative rate” (TNR) refers to the number of true negatives divided by the sum of the number of true negatives and false positives. Specificity can characterize the ability of an assay or method to correctly identify a proportion of the population that truly does not have a condition. For example, specificity can characterize the ability of a method to correctly identify the number of subjects within a population not having cancer. In another example, specificity can characterize the ability of a method to correctly identify one or more markers indicative of cancer.

As used herein, the term “subject” refers to any living or non-living organism, including but not limited to a human (e.g., a male human, female human, fetus, pregnant female, child, or the like), a non-human animal, a plant, a bacterium, a fungus or a protist. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a male or female of any stage (e.g., a man, a women or a child).

As used herein, the term “tissue” can correspond to a group of cells that group together as a functional unit. More than one type of cell can be found in a single tissue. Different types of tissue may consist of different types of cells (e.g., hepatocytes, alveolar cells or blood cells), but also can correspond to tissue from different organisms (mother vs. fetus) or to healthy cells vs. tumor cells. The term “tissue” can generally refer to any group of cells found in the human body (e.g., heart tissue, lung tissue, kidney tissue, nasopharyngeal tissue, oropharyngeal tissue). In some aspects, the term “tissue” or “tissue type” can be used to refer to a tissue from which a cell-free nucleic acid originates. In one example, viral nucleic acid fragments can be derived from blood tissue. In another example, viral nucleic acid fragments can be derived from tumor tissue.

As used herein, the term “true negative” (TN) refers to a subject that does not have a condition or does not have a detectable condition. True negative can refer to a subject that does not have a disease or a detectable disease, such as a tumor, a cancer, a precancerous condition (e.g., a precancerous lesion), a localized, or a metastasized cancer, a non-malignant disease, or a subject that is otherwise healthy. True negative can refer to a subject that does not have a condition or does not have a detectable condition, or is identified as not having the condition by an assay or method of the present disclosure.

As used herein, the term “APOBEC” refers to an enzyme in a family of cytidine deaminases. See Smith et al., 2012, Semin Cell Dev Biol 23(3): 258-268. Cytidine deaminases are responsible for multiple maintenance processes of DNA, and are induced by cytokines associated with the inflammatory response. See Siriwardena et al., 2016, Chem Rev 116(20): 12688-12710. APOBEC enzymes play important roles in gene regulation during the inflammatory response and are involved in the response to various pathogens. APOBEC activity can also result in somatic hypermutation, which in some circumstances is beneficial in providing variability in antibodies generated by cells. However, in some cases, APOBEC-associated mutations (referred to as APOBEC induced mutational signatures herein) have been linked to the presence of cancers. See Seplyarskiy et al., 2016, Genome Res 26(2): 174-182. In particular, mutation signature types 2 and 13 are highly correlated with different cancers. See Alexandrov et al., 2013, Nature, 500(7463), 415-421. Further, the expression levels of certain members of the APOBEC protein family have also been correlated to cancer. See Wang et al., 2018, Oncogene 37:3924-3936.

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

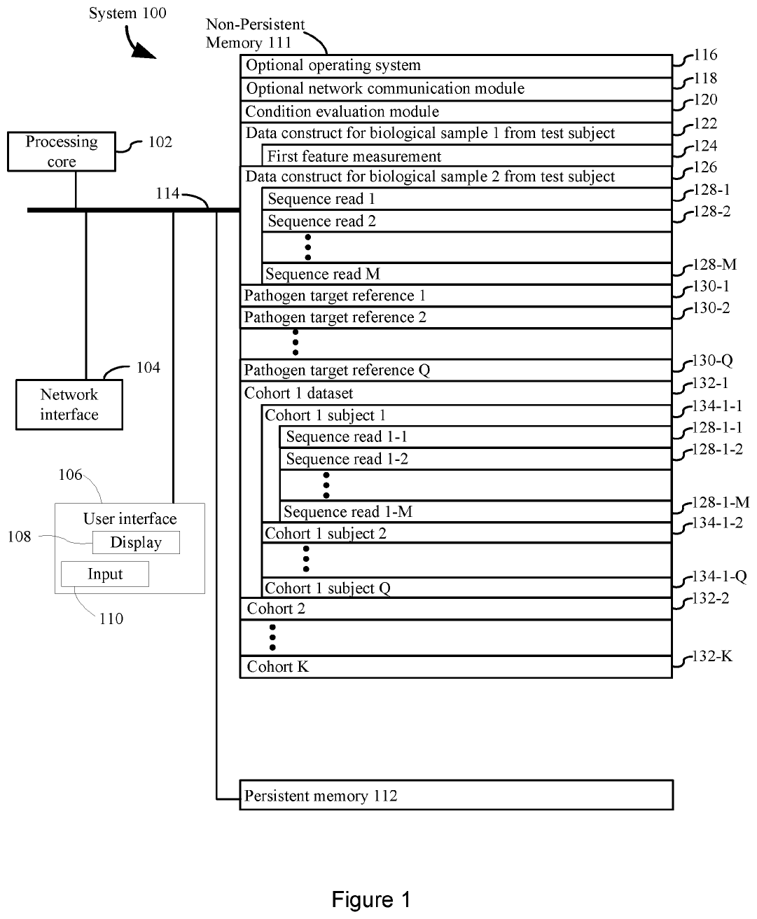

Exemplary System Embodiments. Details of an exemplary system are now described in conjunction with FIG. 1. FIG. 1 is a block diagram illustrating a system 100 in accordance with some implementations. The device 100 in some implementations includes one or more processing units CPU(s) 102 (also referred to as processors), one or more network interfaces 104, a user interface 106, a non-persistent memory 111, a persistent memory 112, and one or more communication buses 114 for interconnecting these components. The one or more communication buses 114 optionally include circuitry (sometimes called a chipset) that interconnects and controls communications between system components. The non-persistent memory 111 typically includes high-speed random access memory, such as DRAM, SRAM, DDR RAM, ROM, EEPROM, flash memory, whereas the persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 112 optionally includes one or more storage devices remotely located from the CPU(s) 102. The persistent memory 112, and the non-volatile memory device(s) within the non-persistent memory 112, comprise non-transitory computer readable storage medium. In some implementations, the non-persistent memory 111 or alternatively the non-transitory computer readable storage medium stores the following programs, modules and data structures, or a subset thereof, sometimes in conjunction with the persistent memory 112:
an optional operating system 116, which includes procedures for handling various basic system services and for performing hardware dependent tasks;
an optional network communication module (or instructions) 118 for connecting the system 100 with other devices, or a communication network;
a condition evaluation module 120 for screening for a cancer condition in a test subject;
a data construct 122 for a first biological sample from a test subject, the data construct 122 comprising a first feature measurement 124;
a data construct 126 for a second biological sample from the test subject, the data construct 126 comprising information regarding a plurality of sequence reads 128 measured from cell-free nucleic acid obtained from the second biological sample;
a pathogen target reference 130 for each pathogen (e.g., virus species) in a plurality of pathogens; and
one or more cohort datasets 132, each respective cohort dataset 132 comprising information for a plurality of subjects 134 of the respective cohort dataset including sequence read 128 data.

In various implementations, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. In some implementations, the non-persistent memory 111 optionally stores a subset of the modules and data structures identified above. Furthermore, in some embodiments, the memory stores additional modules and data structures not described above. In some embodiments, one or more of the above identified elements is stored in a computer system, other than that of visualization system 100, that is addressable by visualization system 100 so that visualization system 100 may retrieve all or a portion of such data when needed.

Although FIG. 1 depicts a “system 100,” the figure is intended more as functional description of the various features that may be present in computer systems than as a structural schematic of the implementations described herein. In practice, and as recognized by those of ordinary skill in the art, items shown separately could be combined and some items could be separated. Moreover, although FIG. 1 depicts certain data and modules in non-persistent memory 111, some or all of these data and modules may be in persistent memory 112.

While a system in accordance with the present disclosure has been disclosed with reference to FIG. 1, methods in accordance with the present disclosure are now detailed. It will be appreciated that any of the disclosed methods can make use of any of the assays or algorithms disclosed in U.S. patent application Ser. No. 15/793,830, filed Oct. 25, 2017 and/or International Patent Publication No. PCT/US17/58099, having an International Filing Date of Oct. 24, 2017, each of which is hereby incorporated by reference, in order to determine a cancer condition in a test subject or a likelihood that the subject has the cancer condition. For instance, any of the disclosed methods can work in conjunction with any of the disclosed methods or algorithms disclosed in U.S. patent application Ser. No. 15/793,830, filed Oct. 25, 2017, and/or International Patent Publication No. PCT/US17/58099, having an International Filing Date of Oct. 24, 2017.

I. Detection of pathogen load by itself (e.g., using targeted panel sequencing, whole genome sequencing, or whole genome bisulfite sequencing). One aspect of the present disclosure provides a method of screening for a cancer condition in a test subject based on genetic material that is derived from one or more pathogens. The method comprises obtaining a first biological sample from the test subject. The first biological sample comprises cell-free nucleic acid from the test subject and potentially cell-free nucleic acid from at least one pathogen in a set of pathogens. In the method, the cell-free nucleic acid in the first biological sample is sequenced (e.g., by whole genome sequencing, targeted panel sequencing, or whole genome bisulfite sequencing, etc.) to generate a plurality of sequence reads 128 from the test subject. Further in the method, for each respective pathogen in the set of pathogens, a corresponding amount of the plurality of sequence reads that map to a sequence in a pathogen target reference 130 for the respective pathogen is determined, thereby obtaining a set of amounts of sequence reads. Each respective amount of sequence reads in the set of amounts of sequence reads is for a corresponding pathogen in the set of pathogens. In the methods, the set of amounts of sequence reads is used to determine whether the test subject has the cancer condition or a likelihood that the test subject has the cancer condition. It will be appreciated that the pathogen target reference 130 may have several different sequences. In typical embodiments, the sequence read from the test subject need only map onto one of these sequences in order to count as mapping onto a sequence in the pathogen target reference. Thus, a sequence read 1 from the test subject that maps to a sequence 1 of the pathogen target reference will contribute to the amount of sequence reads that map onto a sequence in the pathogen target reference as will a sequence read 2 from the test subject that maps to a sequence 2 of the pathogen target reference, whereas a sequence read 3 from the test subject that does not map onto any sequence of the pathogen target reference will not contribute to the amount of sequence reads that map onto a sequence in the pathogen target reference.

In some embodiments, the method includes information regarding the presence of APOBEC induced mutational signatures in the test subject.

In some embodiments, the method relies upon a targeted viral panel. That is, in such embodiments, the pathogen target reference 130 for a particular pathogen is limited to a set of sequences from the genome of the respective pathogen. In some embodiments, the pathogen target reference 130 for a particular pathogen is limited to 100 sequences or less, 50 sequences or less, or 25 or less from the genome of the respective pathogen. Thus, in some such embodiments, the pathogen target reference 130 for the respective pathogen consists of a targeted panel of sequences from the reference genome for the respective pathogen and the determining step limits, for a respective pathogen, the mapping of each sequence read in the plurality of sequence reads (from the target subject) to the corresponding targeted panel of sequences from the reference genome of the respective pathogen.

In some embodiments, the pathogen target reference 130 for each of the set of pathogens are pooled together into a single pool and the step of mapping to a sequence in a pathogen target reference 130 for the respective pathogen is performed concurrently across the entire set of pathogens. In some such embodiments, separate counters are used to track matches between sequence reads from the target subject and sequences in the single pool of pathogen sequences.

In some embodiments, the mapping of sequence reads from the test subject to a sequence in a pathogen target reference 130 for a respective pathogen comprises a sequence alignment between (i) one or more sequence reads in the plurality of sequence reads (from the test subject) and (ii) a sequence in the pathogen target reference 130 for the respective pathogen.

In some embodiments, the mapping of sequence reads from the test subject to a sequence in a pathogen target reference 130 for a respective pathogen comprises a comparison of a methylation pattern between (i) a sequence read in one or more of the plurality of sequence reads and (ii) a sequence in the pathogen target reference for the respective pathogen.

In some embodiments, the method relies upon whole genome sequencing. In some such embodiments, the pathogen target reference for the respective pathogen comprises a reference genome of the respective pathogen and the determining, for each respective pathogen in the set of pathogens, a corresponding amount of the plurality of sequence reads that map to a sequence in a pathogen target reference aligns, for the respective pathogen, each sequence read in the plurality of sequence reads using the entire reference genome of the respective pathogen.

In some embodiments, the pathogen target reference 130 for the respective pathogen comprises at least a portion of the reference genome of the respective pathogen (e.g., less than 10 percent of the reference genome, less than 25 percent of the reference genome, less than 50 percent of the reference genome, less than 90 percent of the reference genome, or between 10 percent than 90 percent of the reference genome etc.). In such embodiments, the determining step aligns, for each respective pathogen in the set of pathogens, a corresponding amount of the plurality of sequence reads that map to a sequence in a pathogen target reference 130, for the respective pathogen, each sequence read in the plurality of sequence reads using the entire reference genome of the respective pathogen.

In some embodiments, the method relies upon whole genome bisulfite sequencing. In such embodiments the determining step compares, for each respective pathogen in the set of pathogens, a corresponding amount of the plurality of sequence reads that map to a sequence in a pathogen target reference 130 compares, for the respective pathogen, a methylation pattern of one or more sequence reads in the plurality of sequence reads to a methylation pattern across all or a portion of the reference genome of the respective pathogen.

In some embodiments, the set of pathogens is a single pathogen. In alternative embodiments, the set of pathogens is a plurality of pathogens, and the determining, for each respective pathogen in the set of pathogens, a corresponding amount of the plurality of sequence reads that map to a sequence in a pathogen target reference 130 is performed for each respective pathogen in the plurality of pathogens. In some embodiments, the set of pathogens comprises between 200 and 500 pathogens, between 2 and 50 pathogens, or between 2 and 30 pathogens.

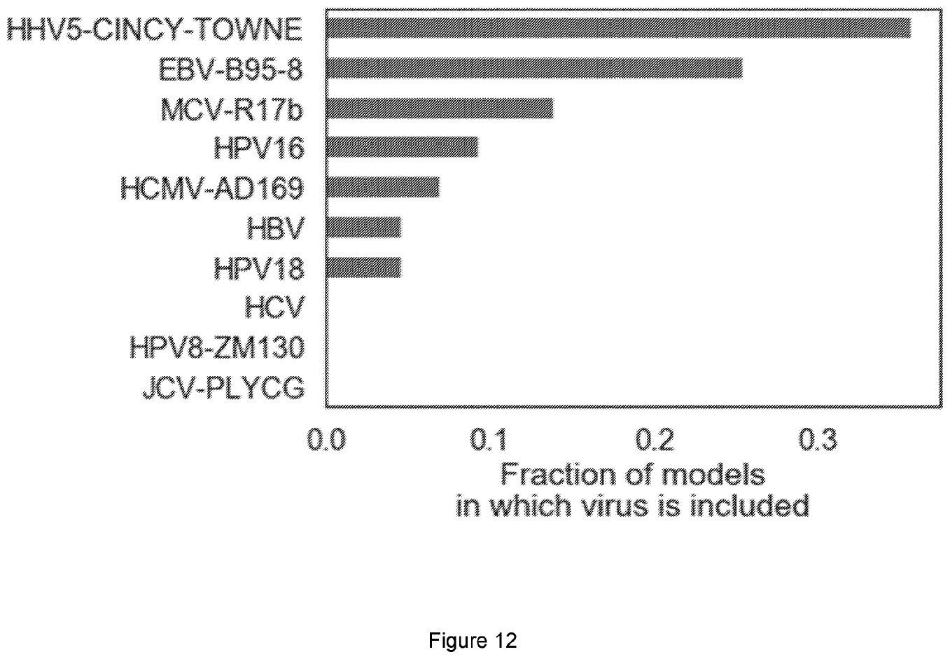

In some embodiments, the set of pathogens comprises or consists of all of the pathogens illustrated in FIG. 12. In some embodiments, the set of pathogens comprises or consists of 2 or more, 3 or more, 4 or more, 5 or more, or 6 or more of the pathogens listed in FIG. 12.

A. Comparing an amount reflecting pathogen load to a reference/cutoff value, in which a training set is used to construct specificity and sensitivity curves. Now that an overview of the methods of the present disclosure have been disclosed, specific embodiments of the methods are described. Accordingly, in some embodiments, the use of the set of amounts of sequence reads to determine whether the test subject has the cancer condition or the likelihood that the test subject has the cancer condition comprises determining a reference amount of sequence reads for a first pathogen in the set of pathogens associated with a predetermined percentile of a first distribution.

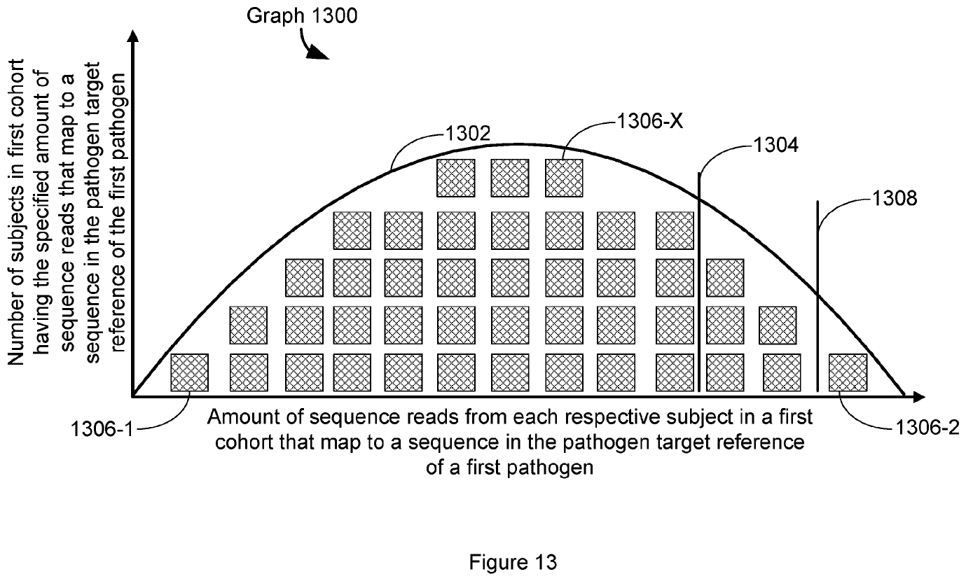

In such embodiments, referring to FIG. 13, each respective subject in a first cohort of subjects contributes to the first distribution 1302 an amount of sequence reads from the respective subject that map to a sequence in the pathogen target reference 130 for the first pathogen. In some such embodiments, this is done by mapping each respective subject in the cohort of subjects onto the X-axis of the graph 1300 based on an amount of sequence reads from the respective subject that map to a sequence in the pathogen target reference 130 for the first pathogen. By mapping all the subjects onto the X-axis in this way, a distribution 1302 is formed where the Y-axis represents a number of subjects and the X-axis represents an amount of sequence reads from each respective subject that map to a sequence in the pathogen target reference 130 for the first pathogen. Thus, in FIG. 13, each box 1306 represents a respective subject in the cohort of subjects. Each respective subject contributes to the first distribution 1302 an amount of sequence reads from the respective subject that map to a sequence in the pathogen target reference 130 for the first pathogen by being placed on the X-axis of graph 1300 at the position that represents the amount of sequence reads from the respective subject that map to a sequence in the pathogen target reference 130 for the first pathogen. Thus subject 1306-1, which has the least amount of sequence reads in the first cohort that map to a sequence in the pathogen target reference 130 for the first pathogen is placed at one end of the distribution 1302 (at a first end of the X-axis) and subject 1306-2, which has the largest amount of sequence reads in the cohort that map to a sequence in the pathogen target reference 130 for the first pathogen, is placed at the other end of the distribution 1302 (at a second end of the X-axis) as illustrated in FIG. 13.

In some embodiments, each subject in a first portion of the first cohort of subjects has the cancer condition, and each subject in a second portion of the first cohort of subjects does not have the cancer condition. In typical embodiments, a biological sample is obtained from each respective subject in the first cohort of subjects and sequence reads are obtained from the first biological sample of the respective subject in the same manner that sequence reads were obtained from the test subject.

What is compared in such embodiments is (i) a first amount that is the amount of the plurality of sequence reads that map to a sequence in the pathogen target reference 130 for the first pathogen from the test subject and (ii) a second amount that is the reference amount of sequence reads for the first pathogen in the set of pathogens associated with the predetermined percentile 1304 of the first distribution. That is, the second amount is taken as the amount of sequence reads at the position of line 1304 in distribution 1302. As an example, if the amount of sequence reads is expressed as a percentage of the sequence reads mapping to the pathogen target reference 130 versus the total number of sequence reads sequenced for a given cohort subject along the X-axis in FIG. 13, then the value for this percentage on the X-axis at line 1304 is used as this second amount (the reference amount of sequence reads for a first pathogen in the set of pathogens associated with a predetermined percentile of a first distribution).

As an example, consider the case where the amount of sequence reads is expressed as a percentage of the sequence reads mapping to the pathogen target reference 130 versus the total number of sequence reads sequenced for a given subject. That is, the X-axis in FIG. 13 denotes percentage of sequence reads. Further still, 3 percent of the plurality of sequence reads from the target subject map to a particular pathogen target reference 130. Further still, each respective subject in the first cohort of subjects contributes to the first distribution 1302 an amount (here a percentage) of sequence reads from the respective subject that map to a sequence in the pathogen target reference 130 for the first pathogen in the manner described above thereby establishing the distribution 1302 shown in FIG. 13. The amount associated with the predetermined percentile 1304 of the first distribution is polled, and in this example is two percent. Thus, the first amount (the percentage of sequence reads mapping to the pathogen target reference 130 from the target subject) exceeds the second amount (the reference percentage of sequence reads associated with the predetermined percentile of distribution 1302) and the test subject is deemed to have the cancer or the likelihood that the test subject has the cancer.

In some embodiments the predetermined percentile of the first distribution is chosen based on a desired target specificity. For instance, in some embodiments, the predetermined percentile of the first distribution (e.g., the position of line 1304 in distribution 1302) is the 80^{th} percentile or greater, the 85^{th} percentile or greater, the 90^{th} percentile or greater, the 95^{th} percentile or greater or the 98^{th} percentile or greater of the distribution 1302. In this way, if the amount of sequence reads mapping to the pathogen target reference 130 from the test subject exceeds this number, it is known that the test subject has an amount of sequence reads mapping to the pathogen target reference 130 that is greater than the predetermined percentile of subjects in the first cohort of subjects. In some embodiments, all of the subjects in the first cohort of subjects have the cancer condition under study.

In some embodiments, rather than just requiring that the amount of sequence reads mapping to the pathogen target reference 130 from the test subject exceed the reference amount of sequence reads associated with the predetermined percentile of the first distribution, the amount of sequence reads mapping to the pathogen target reference 130 from the test subject must exceed the amount of sequence reads associated with the predetermined percentile of the first distribution by a threshold amount in order to make the call that the test subject has the likelihood of having the cancer condition or making the determination that the test subject has the cancer condition. For instance, in some embodiments, in addition to identifying the reference amount of sequence reads for the first pathogen associated with the predetermined percentile of the first distribution, the amount of sequence reads at some distance away from this reference amount in the distribution (e.g., at line 1308) is determined and the amount of sequence reads mapping to the pathogen target reference 130 from the test subject must exceed the amount of sequence reads associated with this position (e.g., at line 1308) of distribution 1302. In some embodiments this distance is one standard deviation, two standard deviations or three standard deviations away from the reference amount of sequence reads in the distribution at line 1304.

Thus, in such embodiments, in addition to determining the reference amount of sequence reads for the first pathogen associated with the predetermined percentile of the first distribution 1302 at line 1304, the amount of sequence reads for the first pathogen associated with 1 standard deviation away from, 2 standard deviations away from, or 3 standard deviations away from this reference amount of sequence reads is made and the amount of sequence reads mapping to the pathogen target reference 130 from the test subject must exceed the amount of sequence reads associated with that point in the distribution 1302 that is one standard deviation away from, two standard deviations away from, or three standard deviations away from this reference amount of sequence reads.

Extension to multiple pathogens. In some embodiments, the method is extended to a plurality of pathogens. In such embodiments, referring to FIG. 13, each respective subject in a first cohort of subjects contributes to the first distribution 1302 an amount of sequence reads from the respective subject that map to a sequence in any pathogen target reference 130 of any pathogen in a plurality of pathogens. In such embodiments, the sequence read from the respective subject need only map onto one of the sequences of one of the pathogen target references in order to count as mapping onto a sequence in the pathogen target reference of any pathogen in the plurality of pathogens. Thus, a sequence read 1 from a subject that maps to a sequence 1 of the pathogen target reference 130-1 will contribute to the amount of sequence reads that map onto a sequence in the pathogen target reference of any of the pathogens as will a sequence read 2 from the test subject that maps to a sequence 1 of the pathogen target reference 130-2, whereas a sequence read 3 from the subject that does not map onto any sequence of any pathogen target reference of the plurality of pathogens will not contribute to the amount of sequence reads that map onto a sequence in any of the pathogen target references.

In some such embodiments, this is done by mapping each respective subject in the cohort of subjects onto the X-axis of the graph 1300 based on an amount of sequence reads from the respective subject that map to a sequence in the pathogen target reference 130 for any pathogen is a plurality of pathogens. By mapping all the subjects onto the X-axis in this way, a distribution 1302 is formed where the Y-axis represents a number of subjects and the X-axis represents an amount of sequence reads from each respective subject that map to a sequence in any pathogen target reference 130 for a plurality of pathogens. Thus, using FIG. 13 as a reference, in such embodiments each box 1306 represents a respective subject in the cohort of subjects. Each respective subject contributes to the first distribution 1302 an amount of sequence reads from the respective subject that map to a sequence in the pathogen target reference 130 for any pathogen in a plurality of pathogens by being placed on the X-axis of graph 1300 at the position that represents the amount of sequence reads from the respective subject that map to a sequence in the pathogen target reference 130 for any pathogen in a plurality of pathogens. Thus subject 1306-1, which has the least amount of sequence reads in the first cohort that map to a sequence in the pathogen target reference 130 for any pathogen in a plurality of pathogens is placed at one end of the distribution 1302 (at a first end of the X-axis) and subject 1306-2, which has the largest amount of sequence reads in the cohort that map to a sequence in the pathogen target reference 130 for any pathogen in the plurality of pathogens, is placed at the other end of the distribution 1302 (at a second end of the X-axis) as illustrated in FIG. 13.

What is compared in such embodiments is (i) a first amount that is the amount of the plurality of sequence reads that map to a sequence in the pathogen target reference 130 of any pathogen in the plurality of pathogens from the test subject and (ii) a second amount that is the reference amount of sequence reads for any pathogen in the plurality of pathogens associated with the predetermined percentile 1304 of the first distribution. That is, the second amount is taken as the amount of sequence reads at the position of line 1304 in distribution 1302. As an example, if the amount of sequence reads is expressed as a percentage of the sequence reads mapping to any pathogen target reference 130 for any pathogen in the plurality of pathogens versus the total number of sequence reads sequenced for a given cohort subject along the X-axis in FIG. 13, then the value for this percentage on the X-axis at line 1304 is used as this second amount (the reference amount of sequence reads mapping to a sequence of the pathogen target reference 130 of any pathogen in the plurality of pathogens associated with a predetermined percentile of a first distribution).

As an example, consider the case where the amount of sequence reads is expressed as a percentage of the sequence reads mapping to the pathogen target reference 130 of any pathogen in the plurality of pathogens versus the total number of sequence reads sequenced for a given subject. That is, the X-axis in FIG. 13 denotes percentage of sequence reads mapping to the sequence of any of the plurality of pathogens. Further still, three percent of the plurality of sequence reads from the target subject map to sequences in the pathogen target references 130 of the plurality of pathogens. Further still, each respective subject in the first cohort of subjects contributes to the first distribution 1302 an amount (here a percentage) of sequence reads from the respective subject that map to a sequence in the pathogen target reference 130 for any of the plurality of pathogens in the manner described above thereby establishing the distribution 1302 shown in FIG. 13. The amount associated with the predetermined percentile 1304 of the first distribution is pooled, and in this example is two percent. Thus, the first amount (the percentage of sequence reads mapping to the pathogen target reference 130 from the target subject) exceeds the second amount (the reference percentage of sequence reads associated with the predetermined percentile of distribution 1302) and the test subject is deemed to have the cancer or the likelihood that the test subject has the cancer.

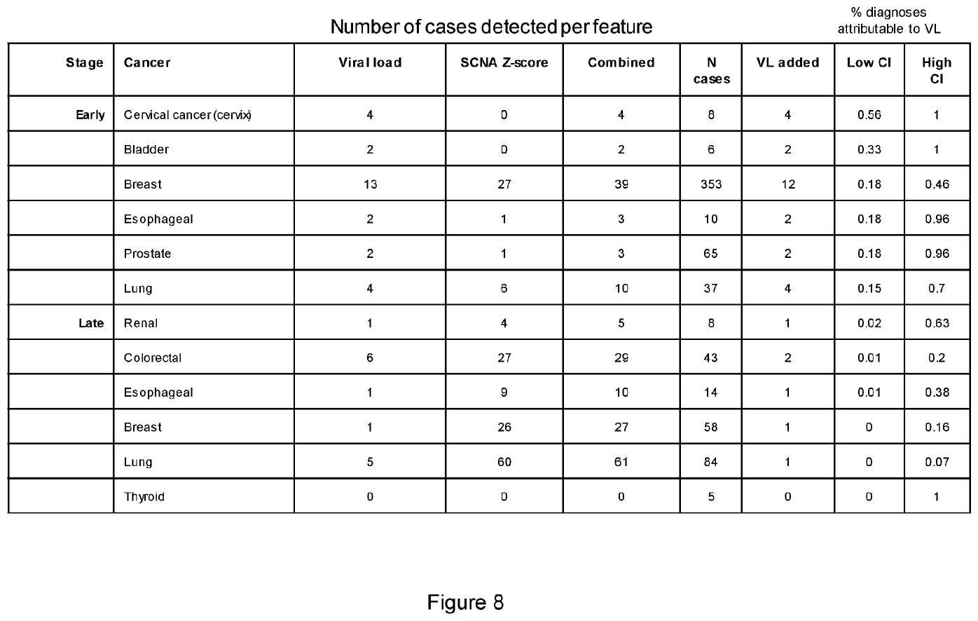

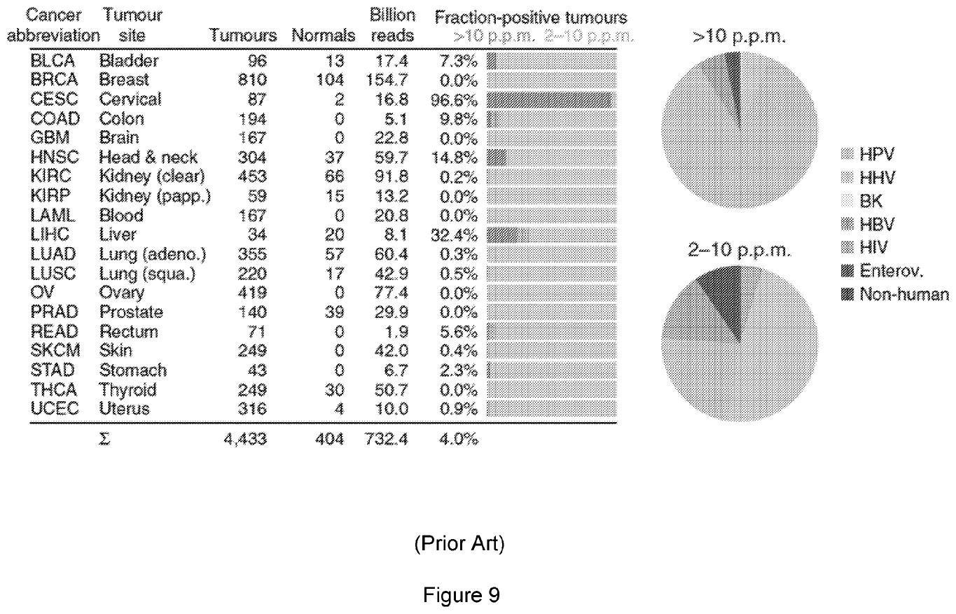

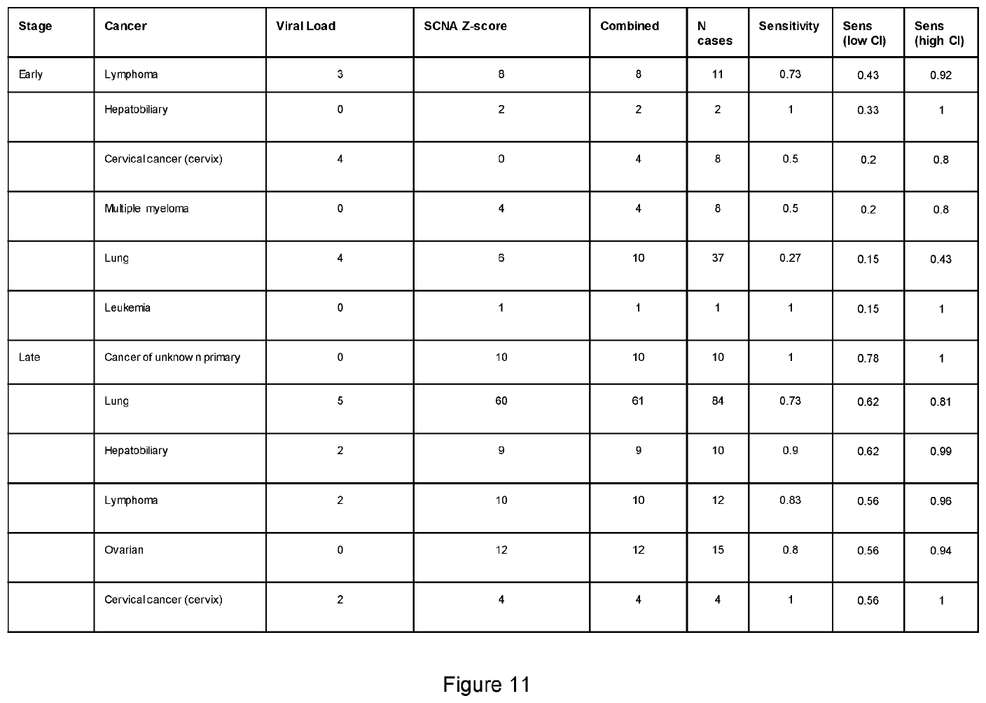

B. Comparing a normalized pathogen load to a reference/cutoff value in which a training set and a control healthy set are used. In some embodiments, pathogen loads are normalized by a certain percentile in the healthy samples in the healthy set to render a normalized viral load for each pathogen type. FIGS. 8 and 11 illustrate the use of viral loads, thresholded as described herein, to determine cancer type and stage. In some embodiments, the normalized loads are then summed to provide an overall pathogen load. The training set is used to construct specificity and sensitivity curves (e.g., where the x-axis represents values of overall pathogen load or a normalized load for a given pathogen). A reference/cutoff value is chosen based on a desired target specificity.

In some such embodiments, the using the set of amounts of sequence reads to determine whether the test subject has the cancer condition or the likelihood that the test subject has the cancer condition comprises determining a reference amount of sequence reads for a first pathogen in the set of pathogens associated with a predetermined percentile of a distribution (e.g., 90%, 95%, 98%, or another suitable percentage). Each respective subject in a cohort of subjects that do not have the cancer condition contributes to the distribution an amount of sequence reads from the respective subject that map to a sequence in the pathogen target reference 130 for the first pathogen.

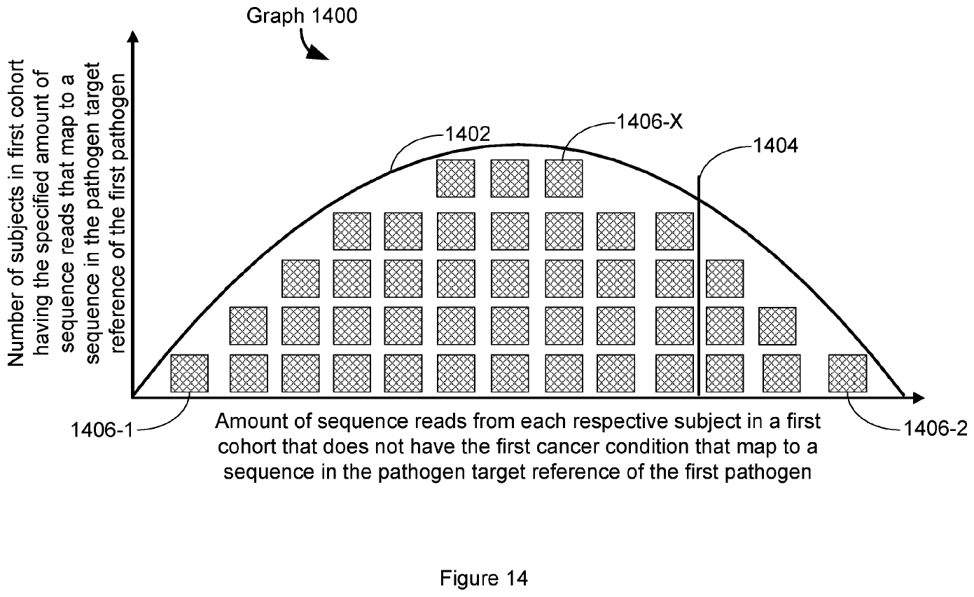

In such embodiments, referring to FIG. 14, each respective subject in the cohort of subjects that do not have the cancer condition contributes to the distribution 1402 an amount of sequence reads from the respective subject that map to a sequence in the pathogen target reference 130 for the first pathogen. In some such embodiments, this is done by mapping each respective subject in the cohort of subjects onto the X-axis of the graph 1400 based on an amount of sequence reads from the respective subject that map to a sequence in the pathogen target reference 130 for the first pathogen. By mapping all the subjects onto the X-axis in this way, a distribution 1402 is formed where the Y-axis represents a number of subjects and the X-axis represents an amount of sequence reads from each respective subject that map to a sequence in the pathogen target reference 130 for the first pathogen. Thus, in FIG. 14, each box 1406 represents a respective subject in the first cohort of subjects. Each respective subject contributes to the first distribution 1402 an amount of sequence reads from the respective subject that map to a sequence in the pathogen target reference 130 for the first pathogen by being placed on the X-axis of graph 1400 at the position that represents the amount of sequence reads from the respective subject that map to a sequence in the pathogen target reference 130 for the first pathogen. Thus subject 1406-1, which has the least amount of sequence reads in the first cohort that map to a sequence in the pathogen target reference 130 for the first pathogen is placed at one end of the distribution 1402 (at a first end of the X-axis) and subject 1406-2, which has the largest amount of sequence reads in the cohort that map to a sequence in the pathogen target reference 130 for the first pathogen, is placed at the other end of the distribution 1402 (at a second end of the X-axis) as illustrated in FIG. 14.

The amount of the plurality of sequence reads that map to a sequence in the pathogen target reference for the first pathogen from the test subject is thresholded (e.g., normalized) by the reference amount of sequence reads for the first pathogen in the set of pathogens associated with the predetermined percentile 1404 of the distribution 1402 to thereby form a scaled amount of the plurality of sequence reads.

For instance, the reference amount is taken as the amount of sequence reads at the position of line 1404 in distribution 1402. As an example, if the amount of sequence reads is expressed as a percentage of the sequence reads mapping to the pathogen target reference 130 versus the total number of sequence reads sequenced for a given cohort subject along the X-axis in FIG. 14, then the value for this percentage on the X-axis at line 1404 is used as this reference amount. For instance, consider the case where the amount of sequence reads is expressed as a percentage of the sequence reads mapping to the pathogen target reference 130 versus the total number of sequence reads sequenced for a given subject. That is, the X-axis in FIG. 14 denotes percentage of sequence reads. Further still, three percent of the plurality of sequence reads from the target subject map to a particular pathogen target reference 130. Further still, each respective subject in the cohort of subjects contributes to the first distribution 1402 an amount (here a percentage) of sequence reads from the respective subject that map to a sequence in the pathogen target reference 130 for the first pathogen in the manner described above thereby establishing the distribution 1402 shown in FIG. 14. The amount associated with the predetermined percentile 1404 of the distribution 1402 is polled, and in this example is two percent. Thus, in this example, the amount of the plurality of sequence reads that map to a sequence in the pathogen target reference for the first pathogen from the test subject (three percent) is thresholded (e.g., normalized) by the reference amount of sequence reads for the first pathogen in the set of pathogens associated with the predetermined percentile of the first distribution (two percent) to thereby form the scaled amount of the plurality of sequence reads (three/two percent, or 1.5 percent).

In typical embodiments, a biological sample is obtained from each respective subject in the first cohort of subjects and sequence reads are obtained from the first biological sample of the respective subject in the same manner that sequence reads were obtained from the test subject. What is compared is (i) the scaled amount of the plurality of sequence reads and (ii) a scaled amount of the plurality of sequence reads associated with a predetermined percentile of a second distribution.

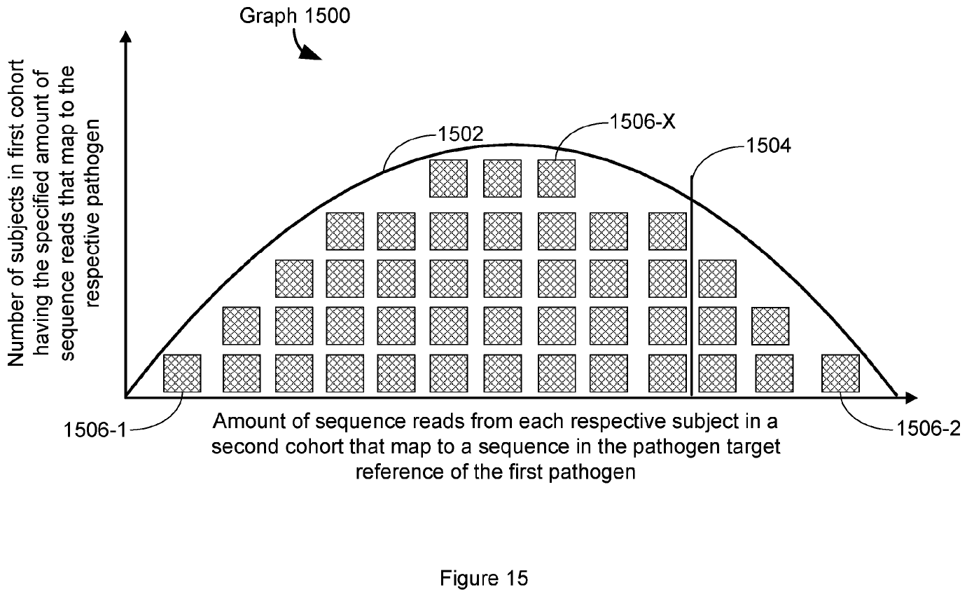

An example of this second distribution is illustrated in FIG. 15. Each respective subject 1506 in the second cohort of subjects contributes to the second distribution 1502 a scaled amount of sequence reads from the respective subject that map to a sequence in the pathogen target reference for the first pathogen. Each subject in a first portion of the subjects in the second cohort have the cancer condition, and each subject in a second portion of the subjects in the second cohort do not have the cancer condition.

In such embodiments, referring to FIG. 15, each respective subject in the second cohort of subjects contributes to the distribution 1502 an amount of sequence reads from the respective subject that map to a sequence in the pathogen target reference 130 for the first pathogen. In some such embodiments, this is done by mapping each respective subject in the second cohort of subjects onto the X-axis of the graph 1500 based on an amount of sequence reads from the respective subject that map to a sequence in the pathogen target reference 130 for the first pathogen.

In alternative embodiments, this is done by mapping each respective subject in the second cohort of subjects onto the X-axis of the graph 1500 based on an amount of sequence reads from the respective subject that map to a sequence in the pathogen target reference 130 for the first pathogen, once this amount has been scaled by the reference amount of sequence reads for the first pathogen associated with the predetermined percentile 1404 of the distribution 1402.

By mapping all the subjects onto the X-axis in this way, the distribution 1502 is formed where the Y-axis represents a number of subjects and the X-axis represents an amount of sequence reads (or a scaled amount of sequence reads) from each respective subject in the second cohort that map to a sequence in the pathogen target reference 130 for the first pathogen. Thus, in FIG. 15, each box 1506 represents a respective subject in the second cohort of subjects. Each respective subject contributes to the second distribution 1502 an amount (or a scaled amount) of sequence reads from the respective subject that map to a sequence in the pathogen target reference 130 for the first pathogen by being placed on the X-axis of graph 1500 at the position that represents the amount (or the scaled amount) of sequence reads from the respective subject that map to a sequence in the pathogen target reference 130 for the first pathogen. Thus subject 1506-1, which has the least amount of sequence reads in the second cohort that map to a sequence in the pathogen target reference 130 for the first pathogen is placed at one end of the distribution 1502 (at a first end of the X-axis) and subject 1506-2, which has the largest amount of sequence reads in the second cohort that map to a sequence in the pathogen target reference 130 for the first pathogen, is placed at the other end of the distribution 1502 (at a second end of the X-axis) as illustrated in FIG. 15.

In some such embodiments, the test subject is deemed to have the cancer condition or the likelihood that the test subject has the cancer condition when the scaled amount of the plurality of sequence reads from the test subject exceeds the scaled amount of plurality of sequence reads associated with a predetermined percentile of the second distribution by a first predetermined cutoff value. For instance, if the predetermined percentile is associated with line 1504, the amount of sequence reads corresponding to line 1504 serves as the scaled amount of plurality of sequence reads associated with a predetermined percentile of the second distribution.

Extension to a plurality of pathogens. In some embodiments, the method is extended to a plurality of pathogens. One way this is done is in some embodiments is to determine a reference amount of sequence reads for each respective pathogen in the plurality of pathogens associated with a predetermined percentile of a corresponding distribution. Each respective subject in a cohort of subjects that do not have the cancer condition contributes to a distribution an amount of sequence reads from the respective subject that map to a sequence in the pathogen target reference 130 for the first pathogen, as discussed with reference to FIG. 14 above. This process is also performed for the second pathogen. For instance, each respective subject in the cohort of subjects that do not have the cancer condition contributes to a distribution similar to that of distribution 1402 of FIG. 14 an amount of sequence reads from the respective subject that map to a sequence in the pathogen target reference 130 for the second pathogen. In some such embodiments, this is done by mapping each respective subject in the cohort of subjects onto the X-axis of a graph like graph 1400 based on an amount of sequence reads from the respective subject that map to a sequence in the pathogen target reference 130 for the second pathogen. By mapping all the subjects onto the X-axis in this way, a distribution is formed where one axis represents a number of subjects and another axis represents an amount of sequence reads from each respective subject that map to a sequence in the pathogen target reference 130 for the second pathogen. The amount of the plurality of sequence reads that map to a sequence in the pathogen target reference for the second pathogen from the test subject is thresholded (e.g., normalized) by the reference amount of sequence reads for the second pathogen associated with the predetermined percentile of the distribution to thereby form a scaled amount of the plurality of sequence reads for the second pathogen.

What is compared in such embodiments is (i) a summation of the scaled amount of the plurality of sequence reads for each pathogen in the plurality of pathogens from the test subject and (ii) a scaled amount associated with a predetermined percentile of a second distribution. For this second distribution, each respective subject in a second cohort of subjects contributes to the second distribution 1502 a summation of a scaled amount that is computed in the same manner as was done for the test subject. That is, the amount of sequence reads from each respective subject in the second cohort that map to a sequence read of the pathogen target reference of a respective pathogen is normalized by the reference amount from the first distribution for the respective pathogen and the summation of the respective scaled amount for the respective subject is contributed to the second distribution. When the summation of the scaled amount of the plurality of sequence reads for each pathogen in the plurality of pathogens from the test subject exceeds the scaled amount of plurality of sequence reads associated with the predetermined percentile of the second distribution, the test subject is deemed to have the cancer condition or the likelihood of having the cancer condition.

C. Using the amounts from each subject in a training set or a normalized pathogen load values from each subject in a training set as input in a binomial or multi-normal classification algorithm. In some such embodiments, the use of the set of amounts of sequence reads to determine whether the test subject has the cancer condition or the likelihood that the test subject has the cancer condition comprises applying the set of amounts of sequence reads to a classifier to thereby determine either (i) whether the test subject has the cancer condition or (ii) the likelihood that test subject has the cancer condition.

In some such embodiments, the classifier is previously trained by inputting into the classifier, for each respective subject in a first cohort of subjects, an amount of sequence reads from the respective subject that map to a sequence in the pathogen target reference for a respective pathogen in the set of pathogens. In some such embodiments, the classifier is previously trained by inputting into the classifier, for each respective subject in a first cohort of subjects, an amount of sequence reads from the respective subject that map to a sequence in the pathogen target reference for each respective pathogen in a plurality of pathogens (e.g., to a sequence that is present in each respective pathogen in the plurality of pathogens). Each subject in a first portion of the subjects in the first cohort has the cancer condition and each subject in a second portion of the subjects in the first cohort does not have the cancer condition.

In alternative embodiments, the classifier is previously trained by inputting into the classifier, for each respective subject in a first cohort of subjects, a normalized amount of sequence reads from the respective subject that map to a sequence in the pathogen target reference for a respective pathogen in the set of pathogens. In such embodiments, each subject in a first portion of the subjects in the first cohort have the cancer condition. Each subject in a second portion of the subjects in the first cohort do not have the cancer condition.

The normalized amount of sequence reads from the respective subject of the first cohort that map to a sequence in the pathogen target reference for the respective pathogen is obtained by normalizing the amount of sequence reads from the respective subject of the first cohort that map to a sequence in the pathogen target reference for the respective pathogen by a reference amount of sequence reads for the respective pathogen associated with a predetermined percentile of a corresponding distribution. Each respective subject in a second cohort of subjects that do not have the cancer condition contributes to the corresponding distribution an amount of sequence reads from the respective subject that map to a sequence in the pathogen target reference for the respective pathogen.

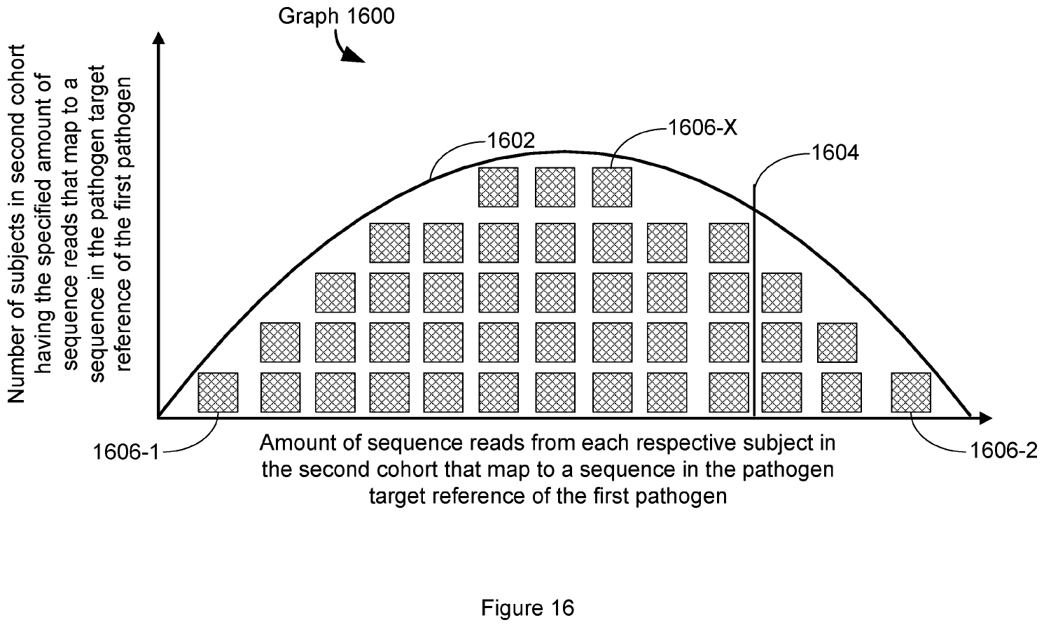

For instance, consider the case where the set of pathogens comprises two pathogens. A normalized amount of sequence reads from the respective subject in the first cohort that map to a sequence in the pathogen target reference for the first pathogen is obtained by normalizing the amount of sequence reads from the respective subject from the first cohort that map to a sequence in the pathogen target reference for the first pathogen by a reference amount of sequence reads for the first pathogen associated with a predetermined percentile of the first distribution 1602 of FIG. 16. Each respective subject in a second cohort of subjects that do not have the cancer condition contributes to the first distribution an amount of sequence reads from the respective subject that map to a sequence in the pathogen target reference for the first pathogen. The reference amount of sequence reads for the first pathogen associated with the predetermined percentile of the first distribution 1602 of FIG. 16 is the amount of sequence reads for the first pathogen at line 1604 of the distribution.

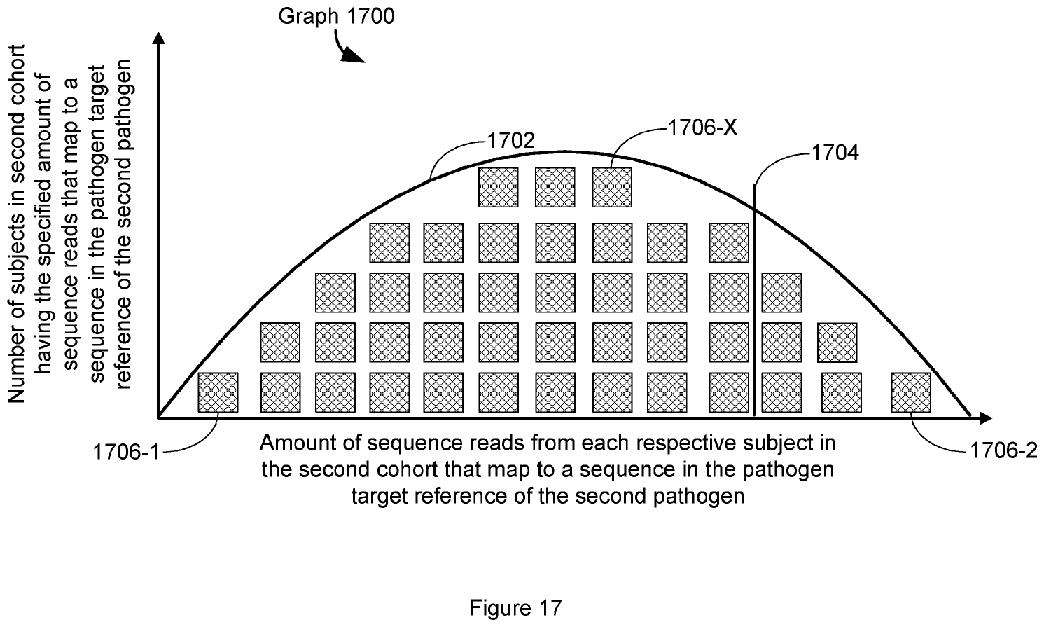

A normalized amount of sequence reads from the respective subject in the first cohort that map to a sequence in the pathogen target reference for the second pathogen is obtained by normalizing the amount of sequence reads from the respective subject from the first cohort that map to a sequence in the pathogen target reference for the second pathogen by a reference amount of sequence reads for the second pathogen associated with a predetermined percentile of the second distribution 1702 of FIG. 17. Each respective subject in the second cohort of subjects that do not have the cancer condition contributes to the second distribution an amount of sequence reads from the respective subject that map to a sequence in the pathogen target reference for the second pathogen. The reference amount of sequence reads for the second pathogen associated with the predetermined percentile of the second distribution 1702 of FIG. 17 is the amount of sequence reads for the second pathogen at line 1704 of the distribution.

Such an approach can be extended for any number of pathogens in the set of pathogens.

In some embodiments, the classifier is a binomial classifier. In some embodiments, the classifier is based on a logistic regression algorithm. In some such embodiments the logistic regression algorithm provides a likelihood that the test subject has or does not have the cancer condition. In some embodiments, the logistic regression algorithm provides a binomial assessment of whether the test subject has or does not have the cancer condition.

In some embodiments, the classifier is a logistic regression algorithm that provides a plurality of likelihoods. Each respective likelihood in the plurality of likelihoods is a likelihood that the test subject has a corresponding cancer condition in a plurality of cancer conditions. Moreover, the plurality of cancer conditions includes the cancer condition.

In some embodiments, the classifier is a multinomial classifier. In some such embodiments, the classifier is based on a logistic regression algorithm, a neural network algorithm, a support vector machine (SVM) algorithm, or a decision tree algorithm.

Logistic regression algorithms are disclosed in Agresti, An Introduction to Categorical Data Analysis, 1996, Chapter 5, pp. 103-144, John Wiley & Son, New York, which is hereby incorporated by reference.

Neural network algorithms, including convolutional neural network algorithms, are disclosed in See, Vincent et al., 2010, J Mach Learn Res 11, pp. 3371-3408; Larochelle et al., 2009, J Mach Learn Res 10, pp. 1-40; and Hassoun, 1995, Fundamentals of Artificial Neural Networks, Massachusetts Institute of Technology, each of which is hereby incorporated by reference.

SVM algorithms are described in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5^{th} Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data training set with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space corresponds to a non-linear decision boundary in the input space. Decision trees are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York. pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, Technical Report 567, Statistics Department, U.C. Berkeley, September 1999, which is hereby incorporated by reference in its entirety.

D. Pathogen load analysis in combination with the presence of a pathogen specific signature for detection of a cancer condition in a test subject. In some embodiments, the method further comprises evaluating the plurality of sequence reads to obtain an indication as to whether a sequence fragment signature associated with a respective pathogen in the set of pathogens is present or absent. In such embodiments, using the set of amounts of sequence reads to determine whether the test subject has the cancer condition or a likelihood that the test subject has the cancer condition uses the indication as to whether the signature fragment signature associated with the respective pathogen is present or absent along with the set of amounts of sequence reads to determine whether the test subject has the cancer condition or the likelihood that test subject has the cancer condition.

Pathogen load analysis in combination with the presence of a methylation signature for detection of a cancer condition. As disclosed herein, the methylation signature can be within the pathogen-derived fragments or test subject derived fragments. In some such embodiments, the method comprises evaluating the plurality of sequence reads to obtain an indication as to whether a methylation signature associated with a first pathogen in the set of pathogens is present or absent. In some such embodiments, the using the set of amounts of sequence reads to determine whether the test subject has the cancer condition or a likelihood that the test subject has the cancer condition uses the indication as to whether the methylation signature associated with the first pathogen is present or absent along with the set of amounts of sequence reads to determine whether the test subject has the cancer condition or the likelihood that test subject has the cancer condition.

In one aspect, pathogen load analysis is performed in combination with the presence of a pathogen specific signature and further in combination with the presence of a methylation signature for cancer detection (e.g., a signature for copy number aberration analysis, a signature for somatic mutation analysis, or a signature for methylation analysis). In some embodiments, the method further comprises evaluating the plurality of sequence reads to obtain an indication as to whether a sequence fragment signature associated with a first pathogen in the set of pathogens is present or absent. Further, the plurality of sequence reads is evaluated to obtain an indication as to whether a methylation signature associated with the first pathogen is present or absent. Further, the using the set of amounts of sequence reads to determine whether the test subject has the cancer condition or a likelihood that the test subject has the cancer condition uses (i) the indication as to whether the sequence fragment signature associated with the first pathogen is present or absent, (ii) an indication as to whether a methylation signature associated with the first pathogen is present or absent, and (iii) the set of amounts of sequence reads to determine whether the test subject has the cancer condition or the likelihood that test subject has the cancer condition.

In some embodiments, the method further comprises performing an assay comprising measuring an amount of a first feature of the cell-free nucleic acid in the first biological sample. In such embodiments, the set of amounts of sequence reads are used to determine whether the test subject has the cancer condition or a likelihood that the test subject has the cancer condition comprises using the amount of the first feature and the set of amounts of sequence reads to determine whether the test subject has the cancer condition or the likelihood that the test subject has the cancer condition.

In some embodiments, an assay is performed that comprises measuring an amount of a first feature of the cell-free nucleic acid in the second biological sample. In such embodiments, the using the set of amounts of sequence reads to determine whether the test subject has the cancer condition or a likelihood that the test subject has the cancer condition comprises using the amount of the first feature and the set of amounts of sequence reads to determine whether the test subject has the cancer condition or the likelihood that the test subject has the cancer condition.

In some embodiments, the cancer condition is cervical, hepatocellular carcinoma, bladder, breast, esophageal, prostate, nasopharyngeal, lung, lymphoma, or leukemia. In some such embodiments, the cancer condition is early stage cancer.

In some embodiments, the cancer condition is renal, hepatocellular carcinoma, colorectal, esophageal, breast, lung, nasopharyngeal, thyroid, lymphoma, ovarian, or cervical. In some such embodiments, the cancer condition is late stage cancer.

In some embodiments, the cancer condition is a liquid cancer, a liver cancer, or lung cancer.

In some embodiments, the first biological sample is plasma. In some embodiments, the first biological sample comprises blood, whole blood, plasma, serum, urine, cerebrospinal fluid, fecal, saliva, sweat, tears, pleural fluid, pericardial fluid, or peritoneal fluid of the test subject. In some embodiments, the first biological sample consists of blood, whole blood, plasma, serum, urine, cerebrospinal fluid, fecal, saliva, sweat, tears, pleural fluid, pericardial fluid, or peritoneal fluid of the test subject.

In some embodiments, a respective pathogen in the set of pathogens is Epstein-Barr virus (EBV), human cytomegalovirus (HCMV), hepatitis B virus (HBV), hepatitis C virus (HCV), human herpes virus (HHV), human mammary tumor virus (HMTV), human papillomavirus 16 (HPV16), human papillomavirus 18 (HPV18), human papillomavirus 60 (HPV-60), human papillomavirus ZM130 (HPV8-ZM130), human T-cell leukemia virus type 1 (HTLV-1), John Cunningham virus (JCV), molluscum contagiosum virus (MCV), or simian vacuolating virus 40 (SV40).

In some embodiments, the set of pathogens is all or a subset of the RefSeq viral genome database. In some embodiments, the set of pathogens comprises any combination of the Epstein-Barr virus (EBV), human cytomegalovirus (HCMV), hepatitis B virus (HBV), hepatitis C virus (HCV), human herpes virus (HHV), human mammary tumor virus (HMTV), human papillomavirus 16 (HPV16), human papillomavirus 18 (HPV18), human papillomavirus 60 (HPV-60), human papillomavirus ZM130 (HPV8-ZM130), human T-cell leukemia virus type 1 (HTLV-1), John Cunningham virus (JCV), molluscum contagiosum virus (MCV), and simian vacuolating virus 40 (SV40).

In some embodiments, the first cohort comprises 20 or 100 subjects. In some embodiments, the first cohort comprises 20 or 100 subjects, and each respective subject in the first cohort contributes a percentage of sequence reads from the respective subject that map to a sequence in the pathogen target reference for the first pathogen to the first distribution.

In some embodiments, the amount of sequence reads from the respective subject that map to a sequence in the pathogen target reference for the respective pathogen is a percentage of the plurality of sequence reads measured from the respective subject that align to a sequence in the pathogen target reference of the respective pathogen.

In some embodiments, the amount of the plurality of sequence reads that map to a sequence in the pathogen target reference for the respective pathogen is a percentage of the plurality of sequence reads from the test subject.

In some embodiments, the amount of sequence reads from the respective subject is a percentage of sequence reads measured from the respective subject that map to a sequence in the pathogen target reference for the first pathogen. In some embodiments, the predetermined percentile of the first distribution is the 95^{th} or 98^{th} percentile. In some embodiments, the first predetermined cutoff value is zero. In some embodiments, the first predetermined cutoff value is a one, two or three standard deviations away from a measure of central tendency of the second distribution.

In some embodiments, the set of pathogens comprises a first pathogen and a second pathogen, and the determining comprises i) determining a first amount of the plurality of sequence reads that map to a sequence in a first pathogen target reference for the first pathogen, and ii) determining a second amount of the plurality of sequence reads that map to a sequence in a second pathogen target reference for the second pathogen. In such embodiments, the method further comprises thresholding the first amount of the plurality of sequence reads from the test subject that map to a sequence in the first pathogen target reference by a first reference amount of sequence reads for the first pathogen associated with a first predetermined percentile of a first distribution to thereby form a scaled first amount of the plurality of sequence reads from the test subject, where each respective subject in a first cohort of subjects that do not have the cancer condition contributes to the first distribution an amount of sequence reads from the respective subject that map to a sequence in the first pathogen target reference for the first pathogen. The method further comprises thresholding the second amount of the plurality of sequence reads from the test subject that map to a sequence in the second pathogen target reference by a second reference amount of sequence reads for the second pathogen associated with a second predetermined percentile of a second distribution to thereby determine a scaled second amount of the plurality of sequence reads from the test subject, where each respective subject in a second cohort of subjects that do not have the cancer condition contributes to the second distribution an amount of sequence reads from the respective subject that map to a sequence in the second pathogen target reference for the second pathogen. In such embodiments, the using the set of amounts of sequence reads to determine whether the test subject has the cancer condition or a likelihood that the test subject has the cancer condition deems the test subject to have the cancer condition or the likelihood that the test subject has the cancer condition when a classifier inputted with at least the scaled first amount and the scaled second amount indicates that the test subject has the cancer condition. In some such embodiments, the classifier is based on a logistic regression algorithm, where the logistic regression individually weights the scaled first amount based on an amount of sequence reads mapping to a sequence in the first pathogen target reference observed in a training cohort of subjects that includes subjects that have the cancer condition and subjects that do not have the cancer condition, and the logistic regression individually weights the scaled second amount based on an amount of sequence reads mapping to a sequence in the second pathogen target reference observed in the training cohort.

In some embodiments, the determining step comprises thresholding the corresponding amount of the plurality of sequence reads that map to a sequence in the pathogen target reference for the respective pathogen based on an amount of sequence reads associated with a predetermined percentile of a respective distribution. Each respective subject in a respective cohort of subjects that do not have the cancer condition contributes to the respective distribution an amount of sequence reads from the respective subject that map to a sequence in the pathogen target reference for the respective pathogen, thereby determining a scaled respective amount of the plurality of sequence reads from the test subject. In such embodiments, the using the set of amounts of sequence reads to determine whether the test subject has the cancer condition or a likelihood that the test subject has the cancer condition deems the test subject to have the cancer condition or the likelihood that the test subject has the cancer condition when a classifier inputted with at least each scaled respective amount of the plurality of sequence reads from the test subject indicates that the test subject has the cancer condition. In some such embodiments, the classifier is based on a logistic regression algorithm that individually weights each scaled respective amount of the plurality of sequence reads based on a corresponding amount of sequence reads mapping to a sequence in the pathogen target reference of the corresponding pathogen observed in a training cohort of subjects that includes subjects that have the cancer condition and subjects that do not have the cancer condition. In some such embodiments, the set of pathogens comprises between 2 and 100 pathogens.

In some embodiments, the classifier is based on a logistic regression algorithm, a neural network algorithm, a support vector machine algorithm, or a decision tree algorithm that has been trained on a training cohort of subjects that includes subjects that have the cancer condition and subjects that do not have the cancer condition.

In some embodiments, the determining step comprises thresholding the corresponding amount of the plurality of sequence reads from the test subject that map to a sequence in the pathogen target reference for the respective pathogen on an amount of sequence reads associated with a predetermined percentile of a respective distribution, where each respective subject in a respective cohort of subjects that do not have the cancer condition contributes to the respective distribution an amount of sequence reads from the respective subject that map to a sequence in the pathogen target reference for the respective pathogen, thereby determining a scaled respective amount of the plurality of sequence reads from the test subject. In such embodiments, the using the set of amounts of sequence reads to determine whether the test subject has the cancer condition or a likelihood that the test subject has the cancer condition sums each scaled respective amount of the plurality of sequence reads from the test subject to determine an overall oncopathogen load and indicates that the test subject has the cancer condition or the likelihood that the test subject has the cancer condition when the overall oncopathogen load satisfies a threshold cutoff condition.

In some embodiments, the using the set of amounts of sequence reads to determine whether the test subject has the cancer condition or a likelihood that the test subject has the cancer condition calls the test subject as having the cancer condition or the likelihood that the test subject has the cancer condition when the set of amounts of sequence reads exceeds a threshold cutoff condition that is a predetermined specificity (e.g., 95^{th} percentile) for overall oncopathogen load across the set of pathogens determined for a pool of subjects that do not have the cancer condition.

In some embodiments, the determining a corresponding amount of the plurality of sequence reads that map to a sequence in the pathogen target reference for the respective pathogen comprises translating the plurality of sequence reads from the test subject in a reading frame to form a plurality of translated sequence reads and comparing the plurality of translated sequence reads to a translation of each sequence in the pathogen target reference.

In some embodiments, the determining a corresponding amount of the plurality of sequence reads that map to a sequence in the pathogen target reference for the respective pathogen comprises k-mer matching the plurality of sequence reads from the test subject to the pathogen target reference in nucleic acid, ribonucleic acid, or protein space. Example k-mer analysis is disclosed in Sievers et al., 2017, Genes 8, 122.

In some embodiments, the test subject is human. In some embodiments, the method further comprises performing an end-point analysis of the corresponding amount of the plurality of sequence reads within the human genome. In such embodiments, the using the set of amounts of sequence reads to determine whether the test subject has the cancer condition or a likelihood that the test subject has the cancer condition further uses the end-point analysis to determine whether the test subject has the cancer condition or a likelihood that the test subject has the cancer condition.

In some embodiments, any of the disclosed methods further comprise providing a therapeutic intervention or imaging of the test subject based on the determination of whether the test subject has the cancer condition or the likelihood that the test subject has the cancer condition.

II. Detection of viral load in conjunction with another type of analysis. A method of screening for a cancer condition in a test subject has been disclosed in Section I above. The present section provides additional methods for screening for a cancer condition in a test subject. In this section any of the assays or methods described in Section I is combined with another assay that measures a first feature in a test subject in order to screen for the cancer condition in a test subject. Moreover, the present section provides more details on the types of cancer conditions, types of sequence reads, and other experimental details that can be used in the methods of Section I above.

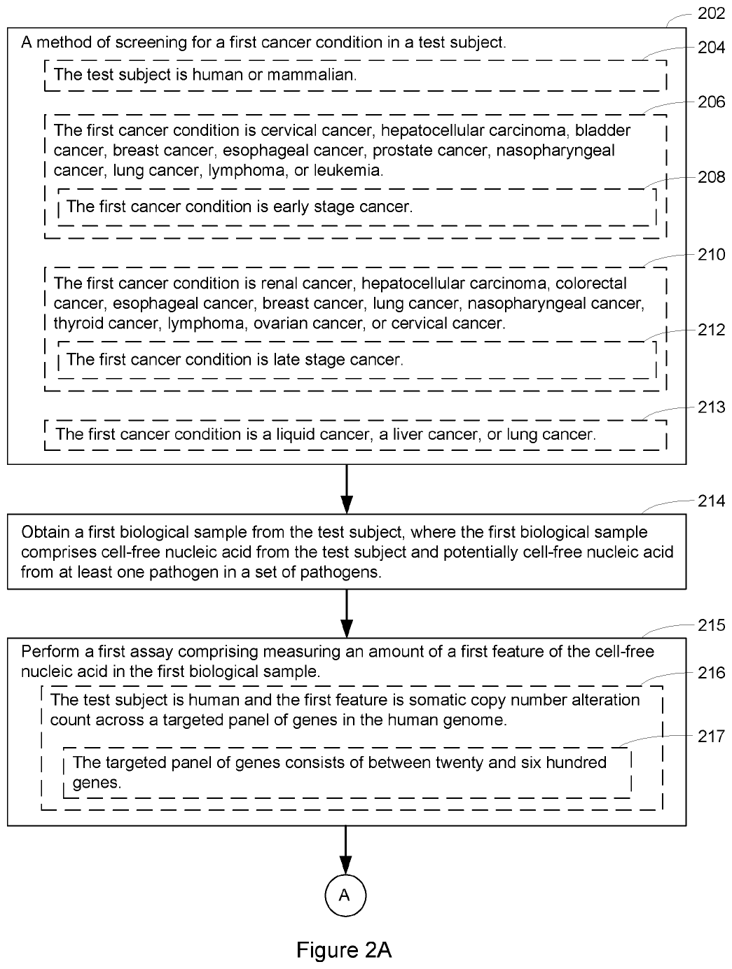

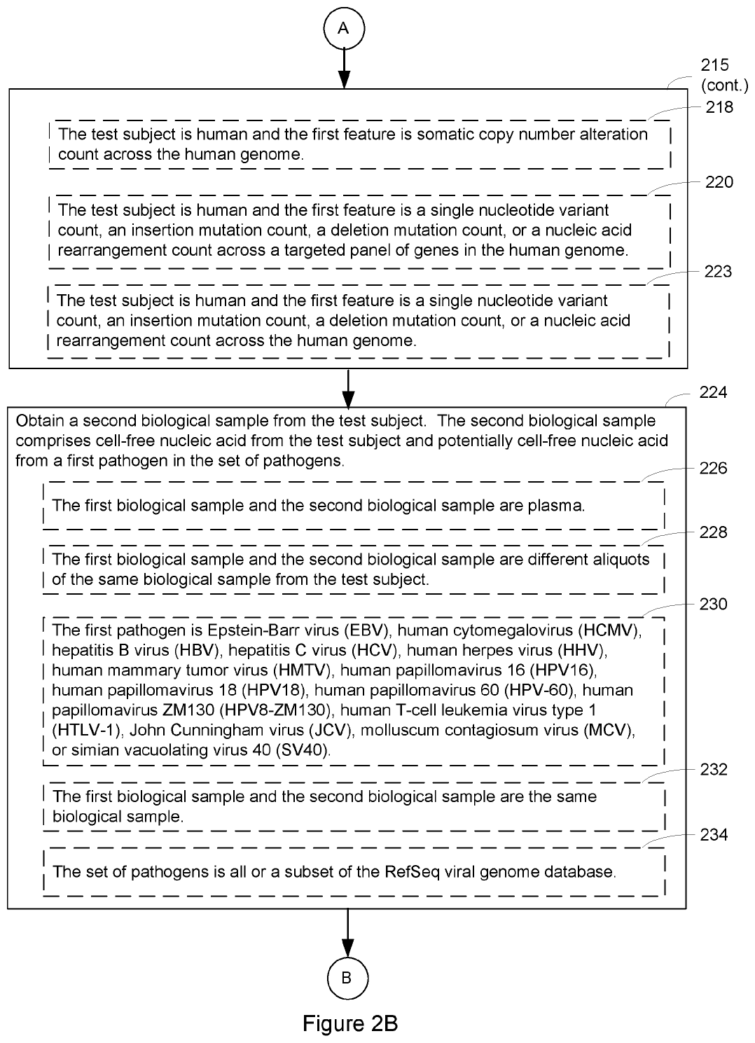

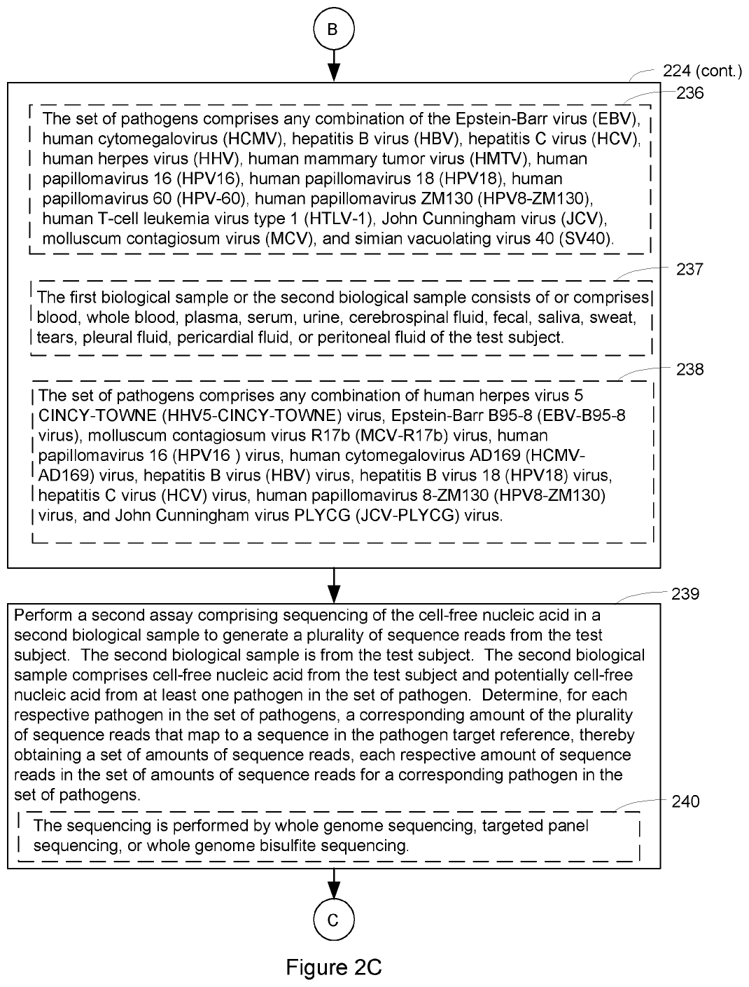

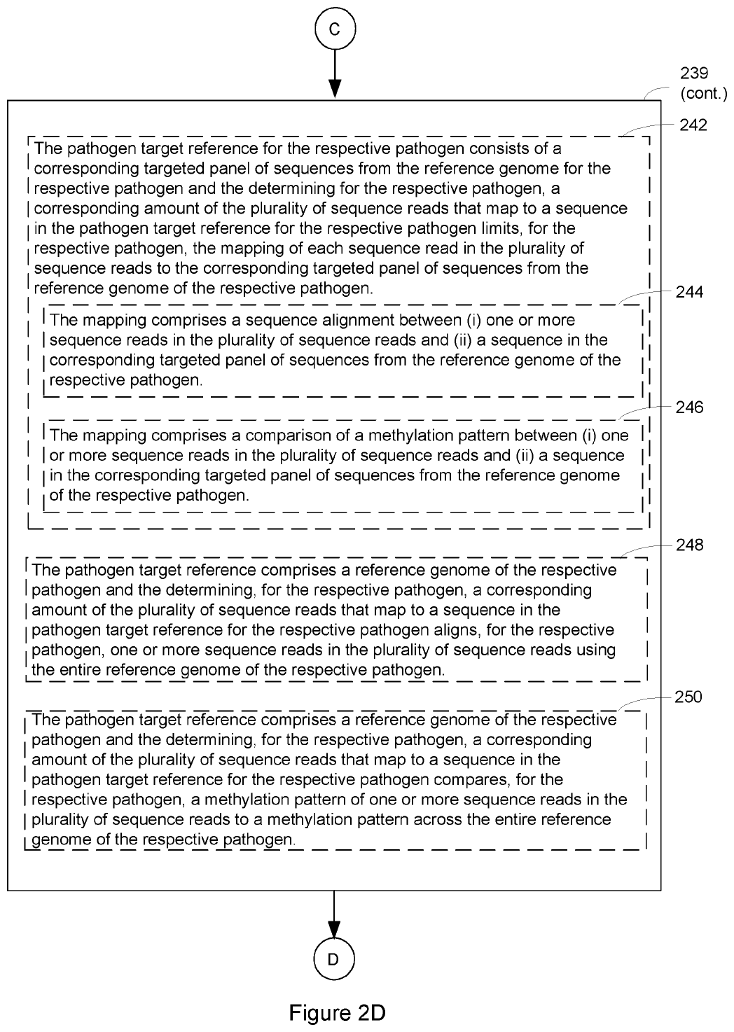

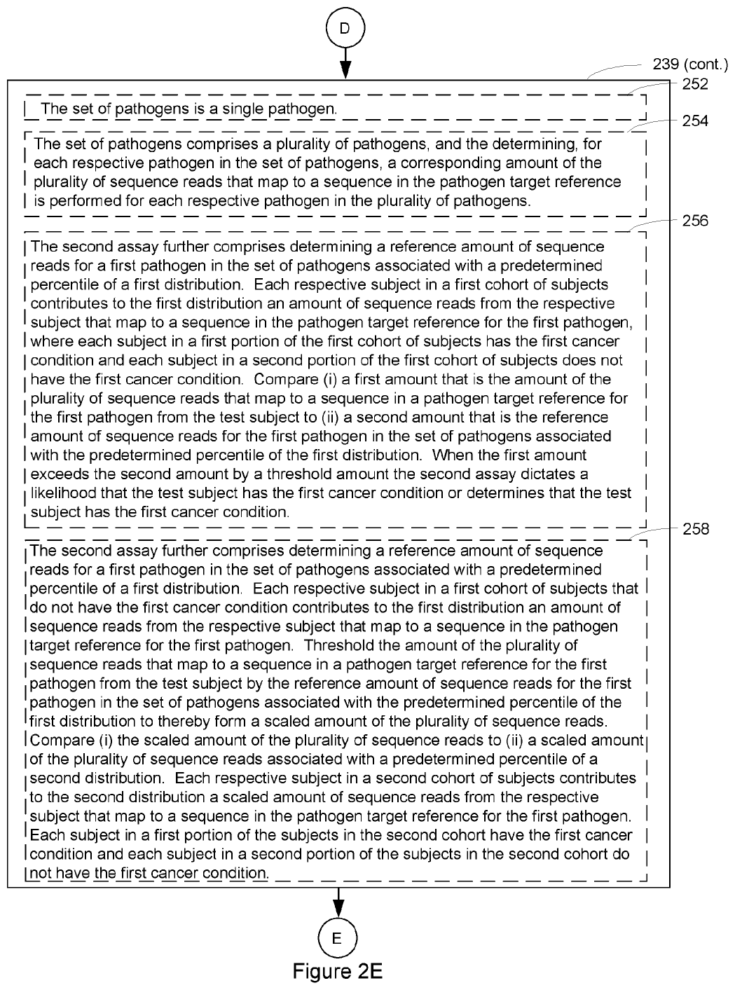

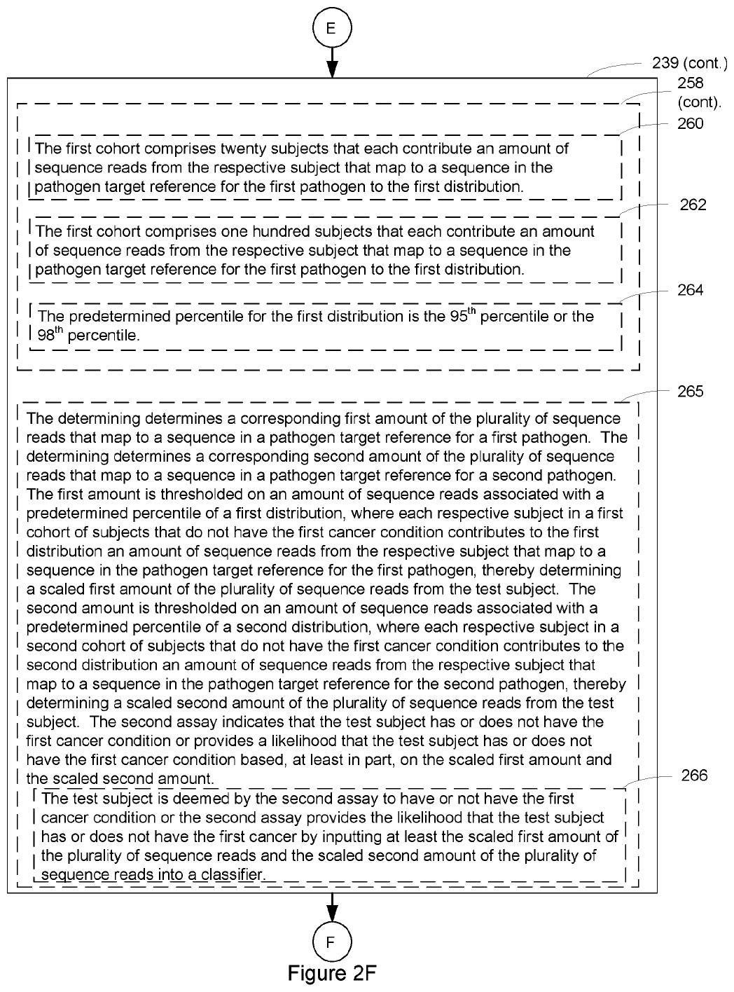

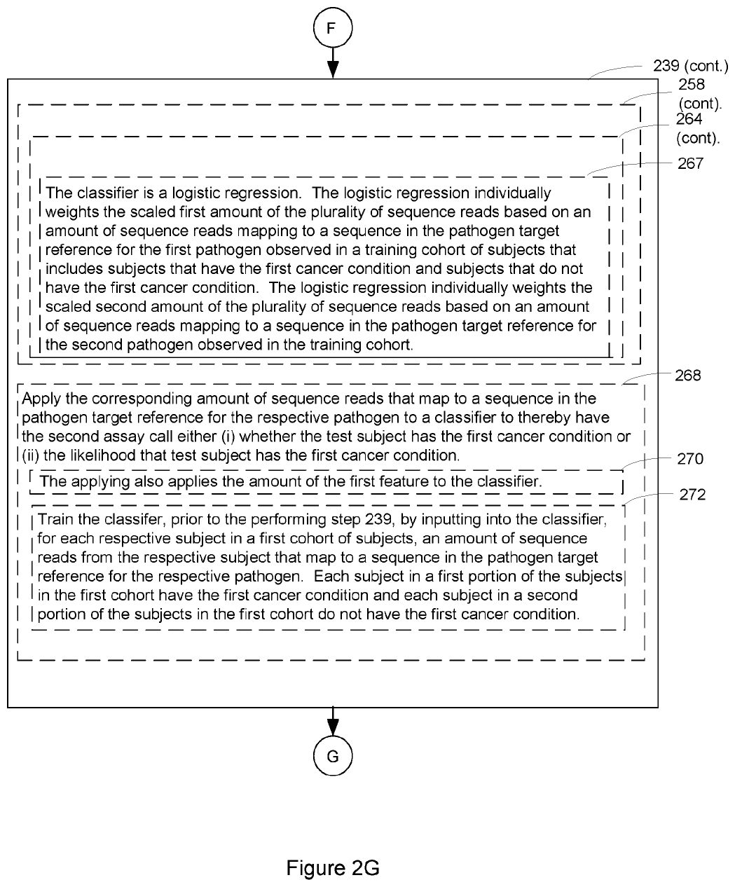

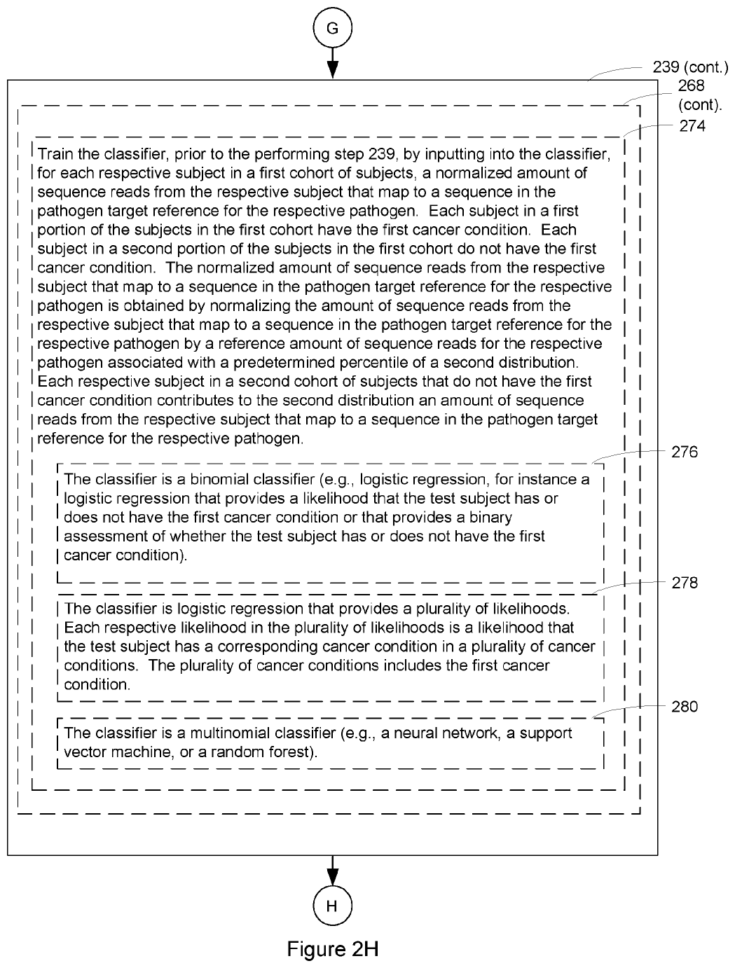

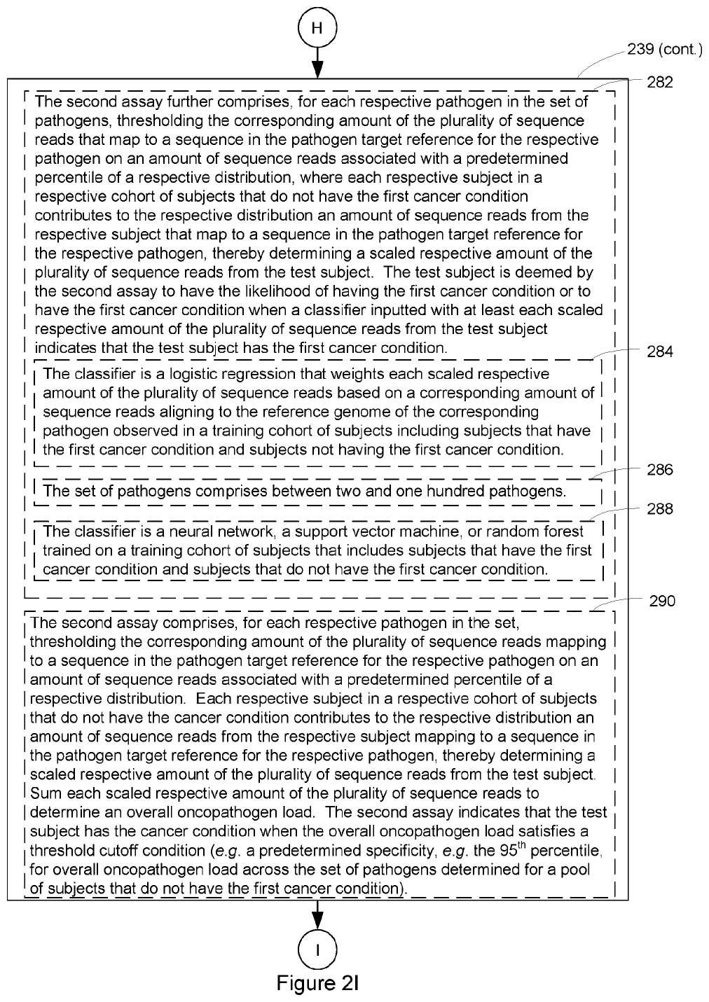

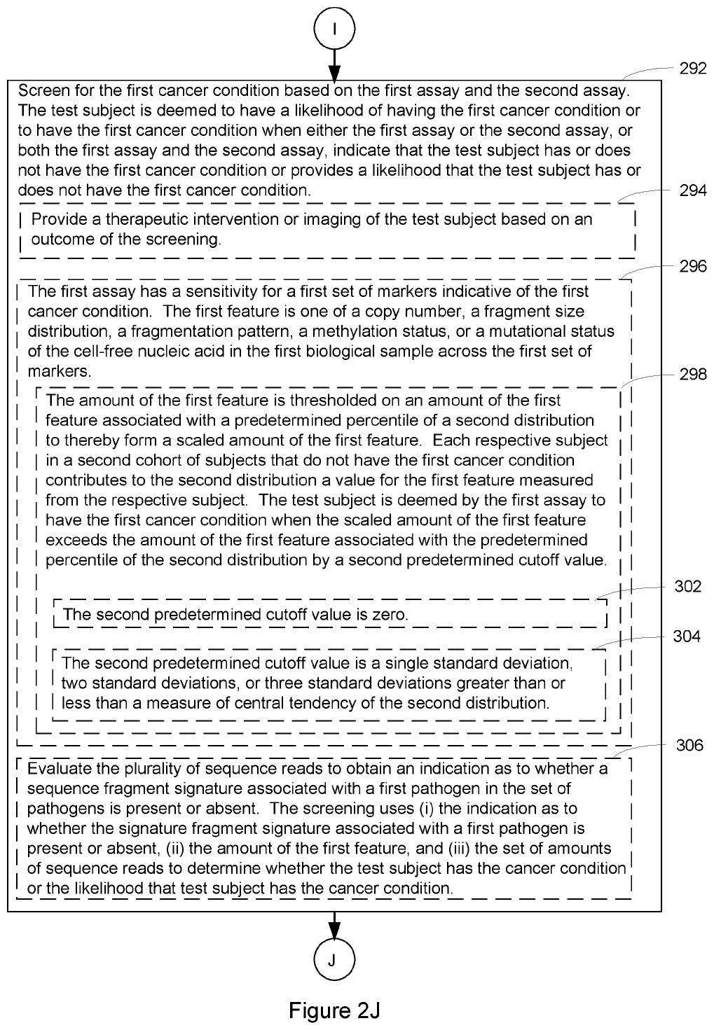

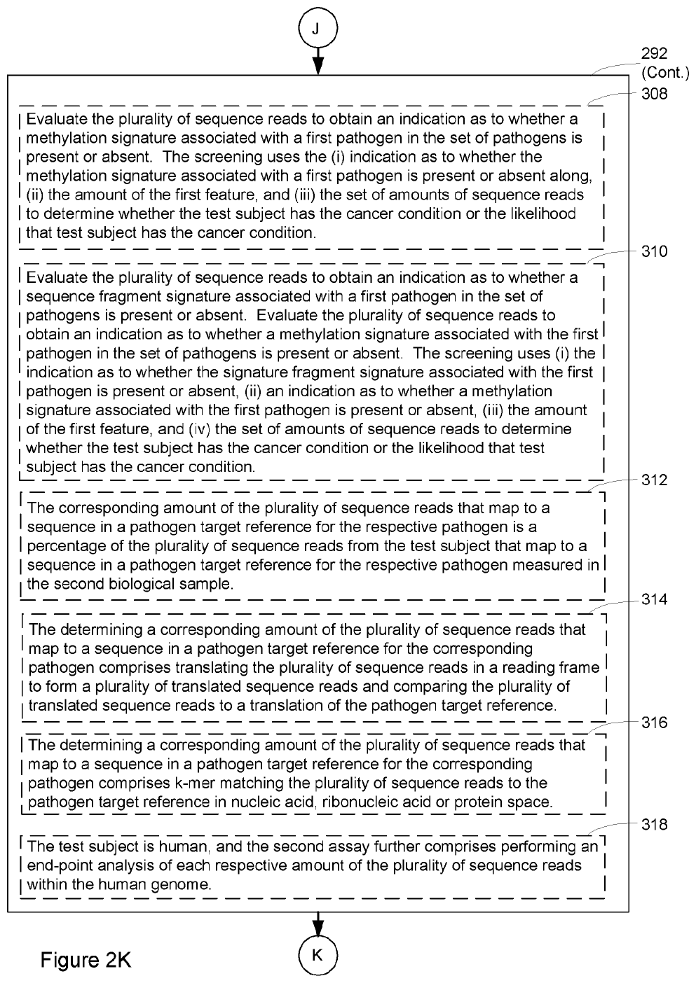

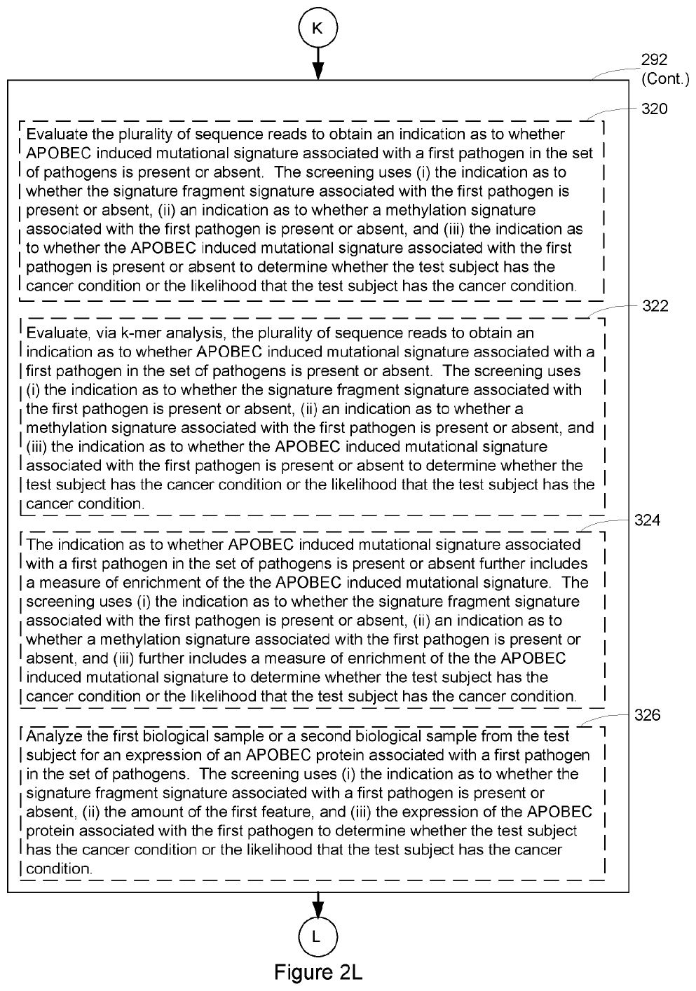

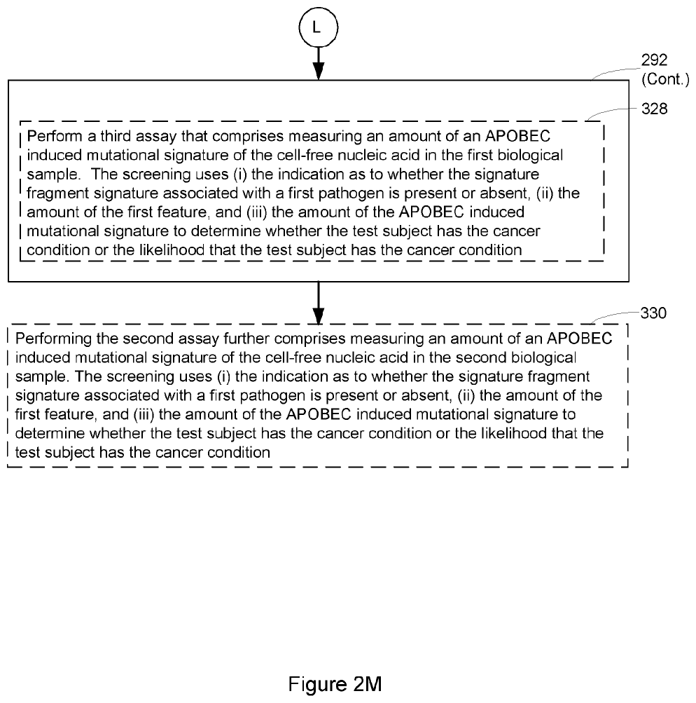

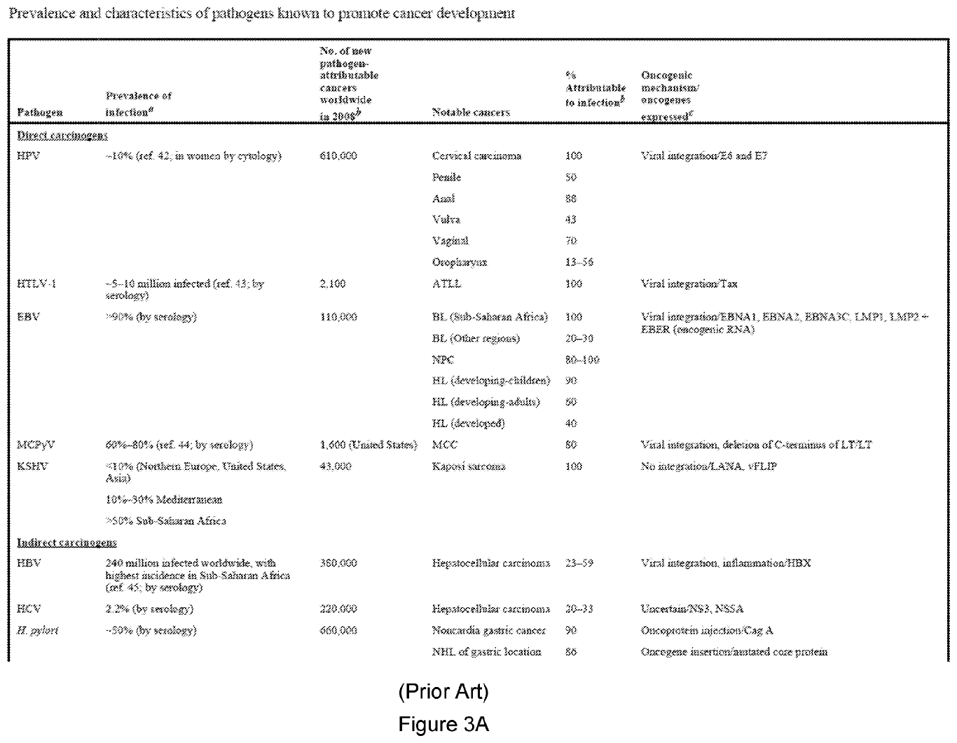

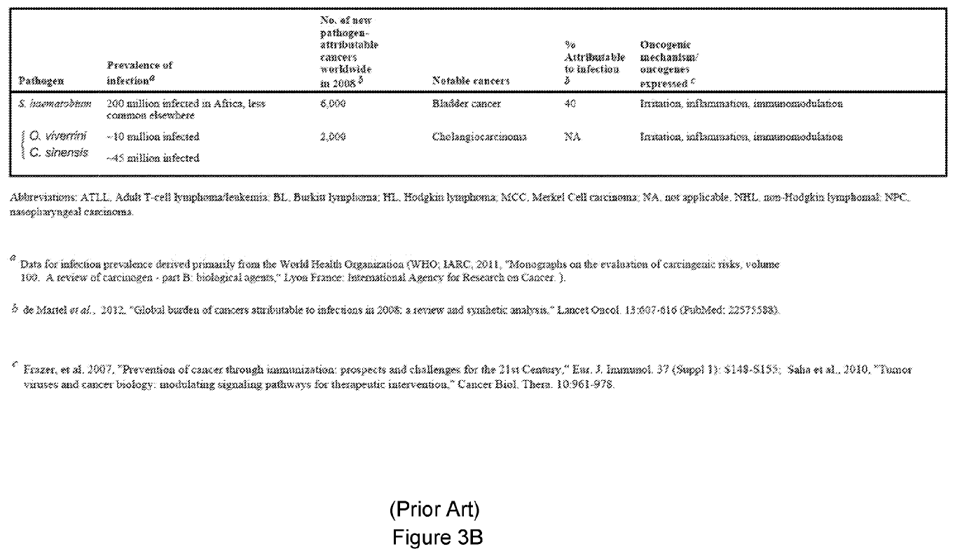

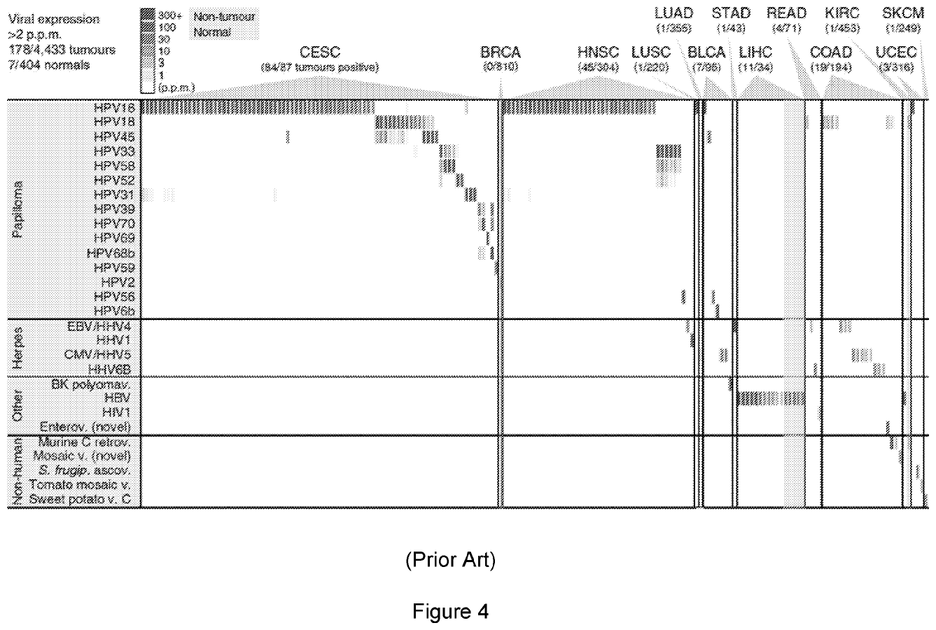

Referring to blocks 202-213 of FIG. 2A, in some embodiments a method of screening for a cancer condition in a test subject is performed at a computer system, such as system 100 of FIG. 1, which has one or more processors 102 and memory 111/112 storing one or more programs, such as condition evaluation module 120, for execution by the one or more processors.

Referring to block 204, in some embodiments the test subject is human. In some embodiments the test subject mammalian. In some embodiments, the test subject is any living or non-living organism, including but not limited to a human (e.g., a male human, female human, fetus, pregnant female, child, or the like), a non-human animal, a plant, a bacterium, a fungus or a protist. In some embodiments, test subject is a mammal, reptile, avian, amphibian, fish (e.g., zebrafish), ungulate, ruminant, bovine (e.g., cattle), equine (e.g., horse), caprine and ovine (e.g., sheep, goat), swine (e.g., pig), camelid (e.g., camel, llama, alpaca), non-human primate (e.g., gorilla, chimpanzee, orangutan, lemur, baboon, etc), ursid (e.g., bear), poultry, dog, cat, mouse, guinea-pig, hamster, rat, dolphin, whale and shark. In some embodiments, the subject is a laboratory or farm animal, or a cellular sample derived from an organism disclosed herein. In some embodiments, the test subject is a male or female of any stage (e.g., a man, a women or a child).

A test subject from whom a sample is taken, or is treated by any of the methods or compositions described herein can be of any age and can be an adult, infant, or child. In some cases, the subject, e.g., patient is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 years old, or within a range therein (e.g., between about 2 and about 20 years old, between about 20 and about 40 years old, or between about 40 and about 90 years old). A particular class of subjects, e.g., patients that can benefit from a method of the present disclosure is subjects, e.g., patients over the age of 40.

Another particular class of subjects, e.g., patients that can benefit from a method of the present disclosure is pediatric patients, who can be at higher risk of chronic heart symptoms. Furthermore, a subject, e.g., patient from whom a sample is taken, or is treated by any of the methods or compositions described herein, can be male or female.

Referring to block 206, in some embodiments, the cancer condition is cervical, hepatocellular, bladder, breast, esophageal, prostate, nasopharyngeal, lung, lymphoma, or leukemia. Referring to block 208 in conjunction with FIG. 11, in some such embodiments the cancer condition is early stage cancer. FIG. 11 discloses the identification of these conditions using the methods of the present disclosure that are disclosed and described in conjunction with FIG. 2.

Referring to block 210, in some embodiments the cancer condition is renal, hepatocellular carcinoma, colorectal, esophageal, breast, lung, nasopharyngeal, thyroid, lymphoma, ovarian cancer, or cervical. Referring to block 212 in conjunction with FIG. 11, in some such embodiments, the cancer condition is late stage cancer. FIG. 11 discloses the identification of these conditions using the methods of the present disclosure that are disclosed and described in conjunction with FIG. 2.

Referring to block 213 of FIG. 2A, in some embodiments the cancer condition is a liquid cancer, a liver cancer, or lung cancer.

Referring to block 214 of FIG. 2A, in the present disclosure a first biological sample is obtained from the test subject. The first biological sample comprises cell-free nucleic acid from the test subject and potentially cell-free nucleic acid from at least one pathogen in a set of pathogens.

In some embodiments, the first biological sample comprises blood, whole blood, plasma, serum, urine, cerebrospinal fluid, fecal, saliva, sweat, tears, pleural fluid, pericardial fluid, or peritoneal fluid of the subject. In such embodiments, the first biological sample may include the blood, whole blood, plasma, serum, urine, cerebrospinal fluid, fecal, saliva, sweat, tears, pleural fluid, pericardial fluid, or peritoneal fluid of the subject as well as other components (e.g., solid tissues, etc.) of the subject. A biological sample can be obtained from the test subject invasively (e.g., surgical means) or non-invasively (e.g., a blood draw, a swab, or collection of a discharged sample).

In some embodiments, the biological sample consists of blood, whole blood, plasma, serum, urine, cerebrospinal fluid, fecal, saliva, sweat, tears, pleural fluid, pericardial fluid, or peritoneal fluid of the subject. In such embodiments, the biological sample is limited to blood, whole blood, plasma, serum, urine, cerebrospinal fluid, fecal, saliva, sweat, tears, pleural fluid, pericardial fluid, or peritoneal fluid of the subject and does not contain other components (e.g., solid tissues, etc.) of the subject.

In some embodiments, the biological sample is processed to extract cell-free nucleic acids in preparation for sequencing analysis in any of the manners disclosed in International Patent Application No. PCT/US2019/027756, entitled Systems and Methods for Determining Tumor Fraction in Cell-Free Nucleic Acid,” filed Apr. 16, 2019, which is hereby incorporated by reference.

In some embodiments, the cell-free nucleic acid that is obtained from the first biological sample is in any form of nucleic acid defined in the present disclosure, or a combination thereof. For example, in some embodiments, the cell-free nucleic acid that is obtained from a biological sample is a mixture of RNA and DNA.

Blocks 215-223. Referring to block 215, a first assay is performed that comprises measuring an amount of a first feature of the cell-free nucleic acid in the first biological sample.

Referring to block 216, in some such embodiments the test subject is human and the first feature is somatic copy number alteration count across a targeted panel of genes in the human genome. See, for example, U.S. patent application Ser. No. 13/801,748, filed on Mar. 13, 2013, which is hereby incorporated by reference, for disclosure on determining somatic copy number alteration count. In some embodiments, referring to block 217, the targeted panel of genes consists of between 20 genes and 600 genes.

In some embodiments, the first feature that is measured by the first assay is a single nucleotide variant associated with a predetermined genomic location, an insertion mutation associated with predetermined genomic location, a deletion mutation associated with a predetermined genomic location, a somatic copy number alteration, a nucleic acid rearrangement associated with a predetermined genomic locus, or an aberrant methylation pattern associated with a predetermined genomic location. In some such embodiments, this first feature is identified using any of the methods disclosed in U.S. Pat. App. No. 62/658,479, entitled “Systems and Methods for Classifying Subjects Using Frequencies of Variants In Cell-Free Nucleic Acid,” filed Apr. 16, 2018 which is hereby incorporated by reference.

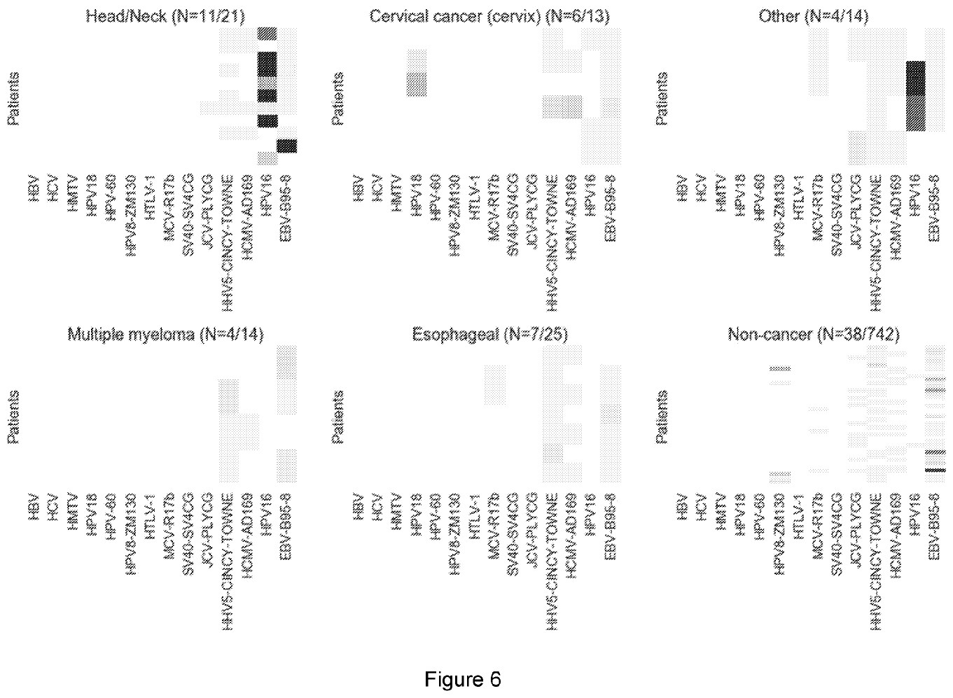

In some embodiments the first feature is associated with a call made by an A score classifier, described herein is a classifier of tumor mutational burden based on targeted sequencing analysis of nonsynonymous mutations. For example, a classification score (e.g., “A score”) can be computed using logistic regression on tumor mutational burden data, where an estimate of tumor mutational burden for each individual is obtained from the targeted cfDNA assay. In some embodiments, a tumor mutational burden can be estimated as the total number of variants per individual that are: called as candidate variants in the cfDNA, passed noise-modeling and joint-calling, and/or found as nonsynonymous in any gene annotation overlapping the variants. The tumor mutational burden numbers of a training set can be fed into a penalized logistic regression classifier to determine cutoffs at which 95% specificity is achieved using cross-validation. An example of the cross-validated performance is shown in FIG. 6. Additional details on A score can be found, for example, in Chaudhary et al., 2017, Journal of Clinical Oncology, 35(5), suppl. e14529, which is hereby incorporated by reference herein in its entirety.

In some embodiments, the first feature is associated with a call made by a B score classifier described in U.S. Pat. App. No. 62/642,461, entitled “Method and System for Selecting, Managing, and Analyzing Data of High Dimensionality,” filed Mar. 13, 2018, which is hereby incorporated by reference. In accordance with the B score method, a first set of sequence reads of nucleic acid samples from healthy subjects in a reference group of healthy subjects are analyzed for regions of low variability. Accordingly, each sequensce read in the first set of sequence reads of nucleic acid samples from each healthy subject are aligned to a region in the reference genome. From this, a training set of sequence reads from sequence reads of nucleic acid samples from subjects in a training group are selected. Each sequence read in the training set aligns to a region in the regions of low variability in the reference genome identified from the reference set. The training set includes sequence reads of nucleic acid samples from healthy subjects as well as sequence reads of nucleic acid samples from diseased subjects who are known to have the cancer. The nucleic acid samples from the training group are of a type that is the same as or similar to that of the nucleic acid samples from the reference group of healthy subjects. From this it is determined, using quantities derived from sequence reads of the training set, one or more parameters that reflect differences between sequence reads of nucleic acid samples from the healthy subjects and sequence reads of nucleic acid samples from the diseased subjects within the training group. Then, a test set of sequence reads associated with nucleic acid samples comprising cfDNA fragments from a test subject whose status with respect to the cancer is unknown is received, and the likelihood of the test subject having the cancer is determined based on the one or more parameters.

In some embodiments, the first feature is associated with a call made by a M score classifier is described in U.S. Pat. Appl. No. 62/642,480, entitled “Methylation Fragment Anomaly Detection,” filed Mar. 13, 2018, which is hereby incorporated by reference.

In some embodiments, the first feature is obtained from any of the disclosed methods or algorithms in U.S. patent application Ser. No. 15/793,830, filed Oct. 25, 2017, and/or International Patent Publication No. PCT/US17/58099, having an International Filing Date of Oct. 24, 2017, each of which is hereby incorporated by reference. In some embodiments, the targeted panel of genes consists of between 2 and 30 genes, between 5 and 50 genes, between 10 and 100 genes, between 30 and 500 genes, or between 50 and 1000 genes.

Referring to block 218 of FIG. 2B, in some embodiments, the test subject is human and the first feature is somatic copy number alteration count across the human genome. Referring to block 220 of FIG. 2B, in some embodiments, the test subject is human and the first feature is a single nucleotide variant count, an insertion mutation count, a deletion mutation count, or a nucleic acid rearrangement count across a targeted panel of genes in the human genome.

In some such embodiments, the subject is a human and a plurality of sequence reads are taken from the first biological sample as part of a targeted plasma assay. That is, the first biological sample is plasma from the test subject and the sequence reads are compared to a targeted panel of genes of the targeted plasma assay in order to identify variants. In some such embodiments, the targeted panel of genes is between 450 and 500 genes. In some embodiments, the targeted panel of genes is within the range of 500±5 genes, within the range of 500±10 genes, or within the range 500±25 genes. In some embodiments, the sequence reads taken from the first biological sample have at least 50,000× coverage for this targeted panel of genes, at least 55,000× coverage for this targeted panel of genes, at least 60,000× coverage for this targeted panel of genes, or at least 70,000× coverage for this targeted panel of genes. In some such embodiments, the targeted plasma assay looks for single nucleotide variants in the targeted panel of genes, insertions in the targeted panel of genes, deletions in the targeted panel of genes, somatic copy number alterations (SCNAs) in the targeted panel of genes, or re-arrangements affecting the targeted panel of genes. Thus, in some embodiments, referring to block 223 of FIG. 2B, the test subject is human and the first feature is a single nucleotide variant count, an insertion mutation count, a deletion mutation count, or a nucleic acid rearrangement count across the human genome.

In some embodiments, steps are taken to make sure that each sequence read represents a unique nucleic acid fragment in the cell-free nucleic acid in the biological sample. Depending on the sequencing method used, each such unique nucleic acid fragment may be represented by a number of sequence reads (e.g., PCR duplicates) in the initial sequence reads obtained. In typical instances, this redundancy in sequence reads to unique nucleic acid fragments in the cell-free nucleic acid is resolved to arrive at the final plurality of sequence reads used in the methods of the present disclosure using multiplex sequencing techniques such as barcoding so that each sequence read in the final plurliaty of sequences uniquely represents a corresponding unique nucleic acid fragment in the cell-free nucleic acid in the biological sample. See Kircher et al., 2012, Nucleic Acids Research 40, No. 1 e3, which is hereby incorporated by reference, for example disclosure on barcoding. In some embodiments, such mapping allows only perfect matches. In some embodiments, such mapping allows some mismatching. In some embodiments, a program such as Bowtie 2 is used to perform such mapping. See, for example, Langmead and Salzberg, 2012, Nat Methods 9, pp. 357-359, for example disclosure on such mapping. In some embodments, a De Bruijn assembler is used for such mappling. In some targeted dequencing embodiments, noise modelling, joint modelling with white blood cells (WBC), and/or edge variant artifact modelling as disclosed in U.S. patent application Ser. No. 16/201,912, entitled “Models for Targeted Sequencing,” filed Nov. 27, 2018, which is hereby incorporated by reference, is used to arrive at the plurality of sequence reads. In the case of whole genome sequencing, the noise models and heuristic algorithms disclosed in U.S. patent application Ser. No. 16/352,214 entitled “Identifying Copy Number Aberrations,” filed Mar. 13, 2019, are used in some embodiments of the present disclosure in obtaining the plurality of sequence reads.

Blocks 224 through 238. In the disclosed methods, a second biological sample is obtained from the test subject. In some embodiments, only a single biological sample is obtained from the test subject. That is, the first biological sample and the second biological sample are the same (e.g. referring to block 232). In some embodiments, the first biological sample and the second biological sample are different. The second biological sample comprises cell-free nucleic acid from the test subject and potentially cell-free nucleic acid from a first pathogen in the set of pathogens. In some embodiments, referring to block 226 of FIG. 2B, the first biological sample and the second biological sample are plasma from the test subject. Referring to block 228 of FIG. 2B, in some embodiments, the first biological sample and the second biological sample are different aliquots of the same biological sample from the test subject.

Referring to block 230 of FIG. 2B, in some embodiments, the methods of the present disclosure screen for a first pathogen that is Epstein-Barr virus (EBV), human cytomegalovirus (HCMV), hepatitis B virus (HBV), hepatitis C virus (HCV), human herpes virus (HHV), human mammary tumor virus (HMTV), human papillomavirus 16 (HPV16), human papillomavirus 18 (HPV18), human papillomavirus 60 (HPV-60), human papillomavirus ZM130 (HPV8-ZM130), human T-cell leukemia virus type 1 (HTLV-1), John Cunningham virus (JCV), molluscum contagiosum virus (MCV), or simian vacuolating virus 40 (SV40). In some embodiments, the methods of the present disclosure screen for plurality of pathogens where the plurality of pathogens comprises at least two, at least three, at least four, at least five, or at least six pathogens in the set of pathogens consisting of Epstein-Barr virus (EBV), human cytomegalovirus (HCMV), hepatitis B virus (HBV), hepatitis C virus (HCV), human herpes virus (HHV), human mammary tumor virus (HMTV), human papillomavirus 16 (HPV16), human papillomavirus 18 (HPV18), human papillomavirus 60 (HPV-60), human papillomavirus ZM130 (HPV8-ZM130), human T-cell leukemia virus type 1 (HTLV-1), John Cunningham virus (JCV), molluscum contagiosum virus (MCV), and simian vacuolating virus 40 (SV40).

In some embodiments, referring to block 234 of FIG. 2B, the set of pathogens is all or a subset of the RefSeq viral genome database. Referring to block 236, in some embodiments, the set of pathogens comprises any combination of the Epstein-Barr virus (EBV), human cytomegalovirus (HCMV), hepatitis B virus (HBV), hepatitis C virus (HCV), human herpes virus (HHV), human mammary tumor virus (HMTV), human papillomavirus 16 (HPV16), human papillomavirus 18 (HPV18), human papillomavirus 60 (HPV-60), human papillomavirus ZM130 (HPV8-ZM130), human T-cell leukemia virus type 1 (HTLV-1), John Cunningham virus (JCV), molluscum contagiosum virus (MCV), and simian vacuolating virus 40 (SV40). In some embodiments, the set of pathogens is a plurality of pathogens that comprises at least two, at least three, at least four, at least five, or at least six pathogens from the group consisting of the Epstein-Barr virus (EBV), human cytomegalovirus (HCMV), hepatitis B virus (HBV), hepatitis C virus (HCV), human herpes virus (HHV), human mammary tumor virus (HMTV), human papillomavirus 16 (HPV16), human papillomavirus 18 (HPV18), human papillomavirus 60 (HPV-60), human papillomavirus ZM130 (HPV8-ZM130), human T-cell leukemia virus type 1 (HTLV-1), John Cunningham virus (JCV), molluscum contagiosum virus (MCV), and simian vacuolating virus 40 (SV40).

Referring to block 237 of FIG. 2C, and as discussed above, in some embodiments the first or second biological sample consists of or comprises blood, whole blood, plasma, serum, urine, cerebrospinal fluid, fecal, saliva, sweat, tears, pleural fluid, pericardial fluid, or peritoneal fluid of the test subject. Referring to block 238 of FIG. 2C, in some embodiments the set of pathogens comprises any combination of human herpes virus 5 CINCY-TOWNE (HHV5-CINCY-TOWNE) virus, Epstein-Barr B95-8 (EBV-B95-8 virus), molluscum contagiosum virus R17b (MCV-R17b) virus, human papillomavirus 16 (HPV16) virus, human cytomegalovirus AD169 (HCMV-AD169) virus, hepatitis B virus (HBV) virus, hepatitis B virus 18 (HPV18) virus, hepatitis C virus (HCV) virus, human papillomavirus 8-ZM130 (HPV8-ZM130) virus, and John Cunningham virus PLYCG (JCV-PLYCG) virus. In some embodiments the set of pathogens comprises any combination of human herpes virus 5 CINCY-TOWNE (HHV5-CINCY-TOWNE) virus, Epstein-Barr B95-8 (EBV-B95-8 virus), molluscum contagiosum virus R17b (MCV-R17b) virus, human papillomavirus 16 (HPV16) virus, human cytomegalovirus AD169 (HCMV-AD169) virus, hepatitis B virus (HBV) virus, and hepatitis B virus 18 (HPV18) virus. FIG. 12 illustrates how models formed in accordance with the present disclosure were among top score models for identifying a cancer condition in subjects that have such cancer conditions.

Block 239. Referring to block 239 of FIG. 2C a second assay is performed that comprising sequencing of the cell-free nucleic acid in the second biological sample to generate a plurality of sequence reads from the test subject.

The second assay can be performed hours, days, or weeks after the first assay. In one embodiment, the second assay is performed immediately after the first assay. In other embodiments, the second assay is performed within 1, 2, 3, 4, 5, or 6 days, within 1, 2, 3, 4, 5, 6, 7, or 8 weeks, within 3, 4, 5, 6, or 12 months after the first assay, or more than 1 year after the first assay. In a particular example, the second assay is performed within 2 weeks of the first sample. Generally, the second assay is used to improve the specificity with which a tumor or cancer type can be detected in a subject. The time between performing the first assay and the second assay can be determined experimentally. In some embodiments, the method can comprise two or more assays, and both assays use the same sample (e.g., a single sample is obtained from a subject, e.g., a patient, prior to performing the first assay, and is preserved for a period of time until performing the second assay). For example, two tubes of blood can be obtained from a subject at the same time. A first tube is used for a first assay. The second tube is used only if results from the first assay from the subject are positive. The sample is preserved using any method known to a person having skill in the art (e.g., cryogenically). This preservation can be beneficial in certain situations, for example, in which a subject can receive a positive test result (e.g., the first assay is indicative of cancer), and the patient can rather not wait until performing the second assay, opting rather to seek a second opinion.

The time between obtaining a biological sample and performing an assay can be optimized to improve the sensitivity and/or specificity of the assay or method. In some embodiments, a biological sample can be obtained immediately before performing an assay (e.g., a first sample is obtained prior to performing the first assay, and a second sample is obtained after performing the first assay but prior to performing the second assay). In some embodiments, a biological sample is obtained, and stored for a period of time (e.g., hours, days, or weeks) before performing an assay. In some embodiments, an assay is performed on a sample within 1, 2, 3, 4, 5, or 6 days, within 1, 2, 3, 4, 5, 6, 7, or 8 weeks, within 3, 4, 5, 6, or 12 months after obtaining the sample from the subject or more than 1 year after obtaining the sample from the subject.

The second biological sample is from the test subject. The second biological sample comprises cell-free nucleic acid from the test subject and potentially cell-free nucleic acid from at least one pathogen in the set of pathogen. There is determined, for each respective pathogen in the set of pathogens, a corresponding amount of the plurality of sequence reads that map to a sequence in the pathogen target reference, thereby obtaining a set of amounts of sequence reads, each respective amount of sequence reads in the set of amounts of sequence reads for a corresponding pathogen in the set of pathogens. Any of the methods disclosed in Section I above can be used for this second assay and, as such, is incorporated by reference into Section II for disclosure on suitable second assays and methods for scoring such assays for a likelihood that the test subject has the cancer condition or has the cancer condition. Additional details regarding this second assay are provided to supplement the disclosure of Section I. Likewise, the additional details provided in this Section are meant to supplement the disclosure of Section I above in terms of experimental detail.

In some embodiments, more than 1000 or 5000 sequence reads are taken from the second biological sample. In some embodiments, the sequence reads taken from the second biological sample provide a coverage rate of 1× or greater, 2× or greater, 5× or greater, 10× or greater, or 50× or greater for at least 2, 5, 10, 20, 30, 40, 50, 60, 70, 80, 90, 98, or at least 99 percent of the genome of the test subject. In some embodiment, the sequence reads taken from the second biological sample provide a coverage rate of 1× or greater, 2× or greater, 5× or greater, 10× or greater, or 50× or greater for at least 3 genes, at least 5 genes, at least 10 genes, at least 20 genes, at least 30 genes, at least 40 genes, at least 50 genes, at least 60 genes, at least 70 genes, at least 80 genes, at least 90 genes, at least 200 genes, at least 300 genes, at least 400 genes, at least 500 genes or at least 1000 genes of the genome of the test subject.

Referring to block 240 of FIG. 2C, in some embodiments the sequencing is performed by whole genome sequencing, targeted panel sequencing, or whole genome bisulfate sequencing.

In some embodiments, the sequencing is performed by whole genome sequencing and the average coverage rate of the plurality of sequence reads taken from the second biological sample is at least 1×, 2×, 3×, 4×, 5×, 6×, 7×, 8×, 9×, 10×, at least 20×, at least 30×, or at least 40× across the genome of the test subject.

In some embodiments the sequencing is performed by targeted panel sequencing in which in which the sequence reads taken from the second biological sample have at least 50,000× coverage, at least 55,000× coverage, at least 60,000× coverage, or at least 70,000× coverage for this targeted panel of genes. In some such embodiments, the targeted panel of genes is between 450 and 500 genes. In some embodiments, the targeted panel of genes is within the range of 500±5 genes, within the range of 500+10 genes, or within the range 500±25 genes.

In some such embodiments, the whole genome bisulfite sequencing identifies one or more methylation state vectors in accordance with Example 1 below, and as further disclosed in U.S. Pat. App. No. 62/642,480, entitled “Methylation Fragment Anomaly Detection,” filed Mar. 13, 2018, which is hereby incorporated by reference.

In some embodiments, the sequence reads are pre-processed to correct biases or errors using one or more methods such as normalization, correction of GC biases, and/or correction of biases due to PCR over-amplification.

Any form of sequencing can be used to obtain the sequence reads from the cell-free nucleic acid obtained from the biological sample including, but not limited to, high-throughput sequencing systems such as the Roche 454 platform, the Applied Biosystems SOLID platform, the Helicos True Single Molecule DNA sequencing technology, the sequencing-by-hybridization platform from Affymetrix Inc., the single molecule, real-time (SMRT) technology of Pacific Biosciences, the sequencing-by-synthesis platforms from 454 Life Sciences, Illumina/Solexa and Helicos Biosciences, and the sequencing-by-ligation platform from Applied Biosystems. The ION TORRENT technology from Life technologies and nanopore sequencing also can be used to obtain sequence reads 140 from the cell-free nucleic acid obtained from the biological sample.

In some embodiments, sequencing-by-synthesis and reversible terminator-based sequencing (e.g., Illumina's Genome Analyzer; Genome Analyzer II; HISEQ 2000; HISEQ 2500 (Illumina, San Diego Calif.)) is used to obtain sequence reads from the cell-free nucleic acid obtained from the biological sample. In some such embodiments, millions of cell-free nucleic acid (e.g., DNA) fragments are sequenced in parallel. In one example of this type of sequencing technology, a flow cell is used that contains an optically transparent slide with eight individual lanes on the surfaces of which are bound oligonucleotide anchors (e.g., adaptor primers). A flow cell often is a solid support that is configured to retain and/or allow the orderly passage of reagent solutions over bound analytes. In some instances, flow cells are planar in shape, optically transparent, generally in the millimeter or sub-millimeter scale, and often have channels or lanes in which the analyte/reagent interaction occurs. In some embodiments, a cell-free nucleic acid sample can include a signal or tag that facilitates detection. In some such embodiments, the acquisition of sequence reads from the cell-free nucleic acid obtained from the biological sample includes obtaining quantification information of the signal or tag via a variety of techniques such as, for example, flow cytometry, quantitative polymerase chain reaction (qPCR), gel electrophoresis, gene-chip analysis, microarray, mass spectrometry, cytofluorimetric analysis, fluorescence microscopy, confocal laser scanning microscopy, laser scanning cytometry, affinity chromatography, manual batch mode separation, electric field suspension, sequencing, and combination thereof.

In some embodiments, sequence reads are obtained in the manner described in the example assay protocol disclosed in Example 2 below.

In some embodiments the sequence reads obtained in block 239 from cell-free nucleic acid of a biological sample comprise more than ten sequence reads of the cell-free nucleic acid, more than one hundred sequence reads of the cell-free nucleic acid, more than five hundred sequence reads of the cell-free nucleic acid, more than one thousand sequence reads of the cell-free nucleic acid, more than two thousand sequence reads of the cell-free nucleic acid, between more than twenty five hundred sequence reads and five thousand sequence reads of the cell-free nucleic acid, or more than five thousand sequence reads of the cell-free nucleic acid. In some embodiments, each of these sequence reads is of a different portion of the cell-free nucleic acid. In some embodiments one sequence read is of all or a same portion of the cell-free nucleic acid as another sequence read in the first plurality of sequence reads.

A. Making Use of a Targeted Pathogen Panel.

Blocks 244-246. Referring to block 242 of FIG. 2D, in some embodiments, the pathogen target reference for the respective pathogen consists of a corresponding targeted panel of sequences from the reference genome for the respective pathogen and the determining for the respective pathogen, a corresponding amount of the plurality of sequence reads that map to a sequence in the pathogen target reference for the respective pathogen limits, for the respective pathogen, the mapping of each sequence read in the plurality of sequence reads to the corresponding targeted panel of sequences from the reference genome of the respective pathogen.

Referring to block 244, in some embodiments the mapping comprises a sequence alignment between (i) one or more sequence reads in the plurality of sequence reads and (ii) a sequence in the corresponding targeted panel of sequences from the reference genome of the respective pathogen. In some embodiments, a respective sequence read in the plurality of sequence reads is deemed to map to a sequence in the corresponding targeted panel of sequences when the one or more sequence reads contains all or a portion of the sequence in the corresponding targeted panel of sequences.

In some embodiments, the plurality of sequence reads is aligned to each sequence in the corresponding targeted panel of sequences by aligning each sequence read in the plurality of sequence reads to a region in each sequence in the corresponding targeted panel in order to determine whether the sequence read contains all or a portion of the sequence in the corresponding targeted panel. The alignment of a sequence read 140 to a region in the sequence in the corresponding targeted panel involves matching sequences from one or more sequence reads in the plurality of sequence reads to that of the sequence in the corresponding targeted panel of sequences based on complete or partial identity between the sequences. Alignments can be done manually or by a computer algorithm, examples including the Efficient Local Alignment of Nucleotide Data (ELAND) computer program distributed as part of the Illumina Genomics Analysis pipeline. The alignment of a sequence read to a sequence in the corresponding targeted panel of sequence can be a 100% sequence match. In some embodiments, an alignment is less than a 100% sequence match (e.g., non-perfect match, partial match, or partial alignment). In some embodiments, an alignment comprises a mismatch. In some embodiments, an alignment comprises 1, 2, 3, 4, or 5 mismatches. Two or more sequences can be aligned using either strand. In some embodiments a nucleic acid sequence is aligned with the reverse complement of another nucleic acid sequence.

B. Making use of whole genome sequencing. In some embodiments, the pathogen target reference comprises a reference genome of the respective pathogen or a portion thereof, and the determining, for each respective pathogen in the set of pathogens, a corresponding amount of the plurality of sequence reads that map to a sequence in a pathogen target reference for the respective pathogen aligns, for the respective pathogen, one or more sequence reads in the plurality of sequence reads using the entire reference genome of the respective pathogen.

In some embodiments, the determining comprises, for each respective pathogen in the set of pathogens, a corresponding amount of the plurality of sequence reads that map to a sequence in a pathogen target reference for the respective pathogen determines a corresponding first amount of the plurality of sequence reads that map to a sequence in a pathogen target reference for a first pathogen. In some embodiments, the determining, for each respective pathogen in the set of pathogens, a corresponding amount of the plurality of sequence reads that map to a sequence in a pathogen target reference for the respective pathogen determines a corresponding second amount of the plurality of sequence reads that map to a sequence in a pathogen target reference for a second pathogen.

Further, the first amount is thresholded on an amount of sequence reads associated with a predetermined percentile of a first distribution, where each respective subject in a first cohort of subjects that do not have the cancer condition contributes to the first distribution an amount of sequence reads from the respective subject that map to a sequence in the pathogen target reference for the first pathogen, thereby determining a scaled first amount of the plurality of sequence reads from the test subject. The second amount is thresholded on an amount of sequence reads associated with a predetermined percentile of a second distribution, where each respective subject in a second cohort of subjects that do not have the cancer condition contributes to the second distribution an amount of sequence reads from the respective subject that map to a sequence in the pathogen target reference for the second pathogen, thereby determining a scaled second amount of the plurality of sequence reads from the test subject. In such embodiments, the second assay indicates that the test subject has or does not have the cancer condition or provides a likelihood that the test subject has or does not have the cancer condition based, at least in part, on the scaled first amount and the scaled second amount.

C. Making use of whole genome bisulfite sequencing. In some embodiments, the pathogen target reference is a reference genome of the respective pathogen or a portion thereof, and the determining comprises, for each respective pathogen in the set of pathogens, determining a corresponding amount of the plurality of sequence reads that map to a sequence in a pathogen target reference for the respective pathogen compares, for the respective pathogen, a methylation pattern of one or more sequence reads in the plurality of sequence reads to a methylation pattern across the entire reference genome of the respective pathogen.

Referring to block 246, in some embodiments the mapping comprises a comparison of a methylation pattern between (i) one or more sequence reads in the plurality of sequence reads and (ii) a sequence in the corresponding targeted panel of sequences from the reference genome of the respective pathogen. More disclosure on such methylation patterns is found in Example 1 below. See also European Pat. Appl. No. 17202149.5, which is hereby incorporated by reference.

Block 248. Referring to block 248 of FIG. 2D, in some embodiments the pathogen target reference 130 comprises a reference genome of the respective pathogen and the determining, for the respective pathogen, a corresponding amount of the plurality of sequence reads that map to a sequence in the pathogen target reference for the respective pathogen aligns, for the respective pathogen, one or more sequence reads in the plurality of sequence reads using the entire reference genome of the respective pathogen.

In some embodiments, the plurality of sequence reads is aligned to the reference genome of the respective pathogen by aligning each sequence read in the plurality of sequence reads to a region in pathogen target reference genome in order to determine whether the sequence read contains all or a portion of the region in pathogen target reference genome. The alignment of a sequence read to a region in pathogen target reference genome sequence involves matching sequences from one or more sequence reads in the plurality of sequence reads to that of the sequence of the region in pathogen target reference genome based on complete or partial identity between the sequences. Alignments can be done manually or by a computer algorithm, examples including the Efficient Local Alignment of Nucleotide Data (ELAND) computer program distributed as part of the Illumina Genomics Analysis pipeline. The alignment of a sequence read to a region in the pathogen target reference genome can be a 100% sequence match. In some embodiments, an alignment is less than a 100% sequence match (e.g., non-perfect match, partial match, or partial alignment). In some embodiments, an alignment comprises a mismatch. In some embodiments, an alignment comprises 1, 2, 3, 4, or 5 mismatches. Two or more sequences can be aligned using either strand. In some embodiments a nucleic acid sequence is aligned with the reverse complement of another nucleic acid sequence.

Block 250. Referring to block 250, in some embodiments, the pathogen target reference comprises a reference genome of the respective pathogen and the determining, for the respective pathogen, a corresponding amount of the plurality of sequence reads that map to a sequence in the pathogen target reference for the respective pathogen compares, for the respective pathogen, a methylation pattern of one or more sequence reads in the plurality of sequence reads to a methylation pattern across the entire reference genome of the respective pathogen. More disclosure on such methylation patterns is found in Example 1 below.

Block 252-254. Referring to block 252 of FIG. 2E, in some embodiments the set of pathogens is a single pathogen. Referring to block 254, in some embodiments, the set of pathogens comprises a plurality of pathogens, and the determining, for each respective pathogen in the set of pathogens, a corresponding amount of the plurality of sequence reads that map to a sequence in the pathogen target reference is performed for each respective pathogen in the plurality of pathogens.

Block 256. Referring to 256 of FIG. 2E, in some embodiments the second assay further comprises determining a reference amount of sequence reads for a first pathogen in the set of pathogens associated with a predetermined percentile of a first distribution. Each respective subject in a first cohort of subjects contributes to the first distribution an amount of sequence reads from the respective subject that map to a sequence in the pathogen target reference for the first pathogen, where each subject in a first portion of the first cohort of subjects has the cancer condition and each subject in a second portion of the first cohort of subjects does not have the cancer condition. In such embodiments a first amount that is the amount of the plurality of sequence reads that map to a sequence in a pathogen target reference for the first pathogen from the test subject is compared to a second amount that is the reference amount of sequence reads for the first pathogen in the set of pathogens associated with the predetermined percentile of the first distribution. When the first amount exceeds the second amount by a threshold amount the second assay dictates a likelihood that the test subject has the cancer condition or determines that the test subject has the cancer condition.

Block 258. Referring to block 258 of FIG. 2E, in some embodiments the second assay further comprises determining a reference amount of sequence reads for a first pathogen in the set of pathogens associated with a predetermined percentile of a first distribution. Each respective subject in a first cohort of subjects that do not have the cancer condition contributes to the first distribution an amount of sequence reads from the respective subject that map to a sequence in the pathogen target reference for the first pathogen. The amount of the plurality of sequence reads that map to a sequence in a pathogen target reference for the first pathogen from the test subject is thresholded (normalized) by the reference amount of sequence reads for the first pathogen in the set of pathogens associated with the predetermined percentile of the first distribution to thereby form a scaled amount of the plurality of sequence reads. The scaled amount of the plurality of sequence reads is compared to the scaled amount of the plurality of sequence reads associated with a predetermined percentile of a second distribution. Each respective subject in a second cohort of subjects contributes to the second distribution a scaled amount of sequence reads from the respective subject that map to a sequence in the pathogen target reference for the first pathogen. Each subject in a first portion of the subjects in the second cohort have the cancer condition and each subject in a second portion of the subjects in the second cohort do not have the cancer condition.

Blocks 260-264. Referring to blocks 260 and 262 of FIG. F, in some embodiments the first cohort comprises 20 or 100 subjects that each contribute an amount of sequence reads from the respective subject that map to a sequence in the pathogen target reference for the first pathogen to the first distribution. Referring to block 265 of FIG. 2F, in some embodiments the predetermined percentile for the first distribution is the 95^{th} percentile or the 98^{th} percentile.

Blocks 265-267. Referring to block 265 of FIG. 2F, in some embodiments the determining step determines a corresponding first amount of the plurality of sequence reads that map to a sequence in a pathogen target reference for a first pathogen. The determining step determines a corresponding second amount of the plurality of sequence reads that map to a sequence in a pathogen target reference for a second pathogen. The first amount is thresholded on an amount of sequence reads associated with a predetermined percentile of a first distribution, where each respective subject in a first cohort of subjects that do not have the cancer condition contributes to the first distribution an amount of sequence reads from the respective subject that map to a sequence in the pathogen target reference for the first pathogen, thereby determining a scaled first amount of the plurality of sequence reads from the test subject. The second amount is thresholded on an amount of sequence reads associated with a predetermined percentile of a second distribution, where each respective subject in a second cohort of subjects that do not have the cancer condition contributes to the second distribution an amount of sequence reads from the respective subject that map to a sequence in the pathogen target reference for the second pathogen, thereby determining a scaled second amount of the plurality of sequence reads from the test subject. The second assay indicates that the test subject has or does not have the cancer condition or provides a likelihood that the test subject has or does not have the cancer condition based, at least in part, on the scaled first amount and the scaled second amount.

Referring to block 266, in some embodiments the test subject is deemed by the second assay to have or not have the cancer condition or the second assay provides a likelihood that the test subject has or does not have the cancer by inputting at least the scaled first amount of the plurality of sequence reads and the scaled second amount of the plurality of sequence reads into a classifier. As an example, referring to block 267 of FIG. 2G, in some embodiments the classifier is a logistic regression. The logistic regression individually weights the scaled first amount of the plurality of sequence reads based on an amount of sequence reads mapping to a sequence in the pathogen target reference for the first pathogen observed in a training cohort of subjects that includes subjects that have the cancer condition and subjects that do not have the cancer condition. The logistic regression individually weights the scaled second amount of the plurality of sequence reads based on an amount of sequence reads mapping to a sequence in the pathogen target reference for the second pathogen observed in the training cohort.

Blocks 268-272. Referring to block 268, in some embodiments the corresponding amount of sequence reads that map to a sequence in the pathogen target reference for the respective pathogen is applied to a classifier to thereby have the second assay call either (i) whether the test subject has the cancer condition or (ii) the likelihood that test subject has the cancer condition. Referring to block 270 of FIG. 2G, in some embodiments the applying step also applies the amount of the first feature to the classifier. Referring to block 272 of FIG. 2G, in some embodiments the first classifier is trained, prior to the performing step 239, by inputting into the classifier, for each respective subject in a first cohort of subjects, an amount of sequence reads from the respective subject that map to a sequence in the pathogen target reference for the respective pathogen. Each subject in a first portion of the subjects in the first cohort have the cancer condition and each subject in a second portion of the subjects in the first cohort do not have the cancer condition.

Block 274. Referring to block 274, in some embodiments the classifier is trained, prior to the performing step 239, by inputting into the classifier, for each respective subject in a first cohort of subjects, a normalized amount of sequence reads from the respective subject that map to a sequence in the pathogen target reference for the respective pathogen. Each subject in a first portion of the subjects in the first cohort has the cancer condition. Each subject in a second portion of the subjects in the first cohort does not have the cancer condition. The normalized amount of sequence reads from the respective subject that map to a sequence in the pathogen target reference for the respective pathogen is obtained by normalizing the amount of sequence reads from the respective subject that map to a sequence in the pathogen target reference for the respective pathogen by a reference amount of sequence reads for the respective pathogen associated with a predetermined percentile of a second distribution. Each respective subject in a second cohort of subjects that do not have the cancer condition contributes to the second distribution an amount of sequence reads from the respective subject that map to a sequence in the pathogen target reference for the respective pathogen.

Block 276. Referring to block 276 of FIG. 2H, in some embodiments the classifier is a binomial classifier (e.g., logistic regression, for instance a logistic regression that provides a likelihood that the test subject has or does not have the cancer condition or that provides a binary assessment of whether the test subject has or does not have the cancer condition).

Block 278. Referring to block 278 of FIG. 2H, in some embodiments the classifier is logistic regression that provides a plurality of likelihoods. Each respective likelihood in the plurality of likelihoods is a likelihood that the test subject has a corresponding cancer condition in a plurality of cancer conditions. The plurality of cancer conditions includes the cancer condition.

Block 280. Referring to block 280 of FIG. 2H, in some embodiments the classifier is a multinomial classifier (e.g., a neural network algorithm, a support vector machine algorithm, or a decision tree algorithm, etc.).

Blocks 282-288. Referring to block 282 of FIG. 2I, in some embodiments the second assay further comprises, for each respective pathogen in the set of pathogens, thresholding the corresponding amount of the plurality of sequence reads that map to a sequence in the pathogen target reference for the respective pathogen on an amount of sequence reads associated with a predetermined percentile of a respective distribution, where each respective subject in a respective cohort of subjects that do not have the cancer condition contributes to the respective distribution an amount of sequence reads from the respective subject that map to a sequence in the pathogen target reference for the respective pathogen, thereby determining a scaled respective amount of the plurality of sequence reads from the test subject. The test subject is deemed by the second assay to have the likelihood of having the cancer condition or to have the cancer condition when a classifier inputted with at least each scaled respective amount of the plurality of sequence reads from the test subject indicates that the test subject has the cancer condition.

Referring to block 284 of FIG. 2I, in some embodiments the classifier is a logistic regression that weights each scaled respective amount of the plurality of sequence reads based on a corresponding amount of sequence reads aligning to the reference genome of the corresponding pathogen observed in a training cohort of subjects including subjects that have the cancer condition and subjects not having the cancer condition.

Referring to block 286 of FIG. 2I, in embodiments, the set of pathogens comprises between two and one hundred pathogens.

Referring to block 288 of FIG. 2I, in some embodiments the classifier is a neural network algorithm, a support vector machine algorithm, or a decision tree algorithm trained on a training cohort of subjects that includes subjects that have the cancer condition and subjects that do not have the cancer condition.

Block 290. Referring to block 290 of FIG. 2I, in some embodiments the second assay comprises, for each respective pathogen in the set, thresholding the corresponding amount of the plurality of sequence reads mapping to a sequence in the pathogen target reference for the respective pathogen on an amount of sequence reads associated with a predetermined percentile of a respective distribution. Each respective subject in a respective cohort of subjects that do not have the cancer condition contributes to the respective distribution an amount of sequence reads from the respective subject mapping to a sequence in the pathogen target reference for the respective pathogen, thereby determining a scaled respective amount of the plurality of sequence reads from the test subject. Sum each scaled respective amount of the plurality of sequence reads to determine an overall oncopathogen load. The second assay indicates that the test subject has the cancer condition when the overall oncopathogen load satisfies a threshold cutoff condition (e.g. a predetermined specificity, e.g. the 90^{th} percentile, 95^{th} percentile, 98^{th} percentile, 99^{th} percentile or some other suitable percentile, for overall oncopathogen load across the set of pathogens determined for a pool of subjects that do not have the cancer condition).

Block 292-296. Referring to block 292 of FIG. 2J, screening for the cancer condition is based on the first assay and the second assay. In such embodiments, the test subject is deemed to have a likelihood of having the cancer condition or to have the cancer condition when either the first assay or the second assay, or both the first and second assay, indicate that the test subject has or does not have the cancer condition or provides a likelihood that the test subject has or does not have the cancer condition. In some such embodiments, a therapeutic intervention or imaging of the test subject is provided based on an outcome of the screening. Referring to block 296 of FIG. 2J, in some embodiments the first assay has a sensitivity for a first set of markers indicative of the cancer condition. The first feature is one of a copy number, a fragment size distribution, a fragmentation pattern, a methylation status, or a mutational status of the cell-free nucleic acid in the first biological sample across the first set of markers.

Blocks 298-304. Referring to block 298 of FIG. 2J, in some embodiments the amount of the first feature is thresholded on an amount of the first feature associated with a predetermined percentile of a second distribution, thereby forming a scaled amount of the first feature. Each respective subject in a second cohort of subjects that do not have the cancer condition contributes to the second distribution a value for the first feature measured from the respective subject. The test subject is deemed by the first assay to have the cancer condition when the scaled amount of the first feature exceeds the amount of the first feature associated with the predetermined percentile of the second distribution by a second predetermined cutoff value. Referring to block 302, in some embodiments the second predetermined cutoff value is zero. Referring to block 304, in some embodiments the second predetermined cutoff value is a one, two, or three standard deviations greater than or less than a measure of central tendency of the second distribution.

Referring to block 306 of FIG. 2J, in some embodiments, the plurality of sequence reads is evaluated to obtain an indication as to whether a sequence fragment signature associated with a first pathogen in the set of pathogens is present or absent. The screening uses (i) the indication as to whether the signature fragment signature associated with a first pathogen is present or absent, (ii) the amount of the first feature, and (iii) the set of amounts of sequence reads to determine whether the test subject has the cancer condition or the likelihood that test subject has the cancer condition.

Referring to block 308 of FIG. 2K, in some embodiments the plurality of sequence reads is evaluated to obtain an indication as to whether a methylation signature associated with a first pathogen in the set of pathogens is present or absent. The screening uses (i) the indication as to whether the methylation signature associated with a first pathogen is present or absent, (ii) the amount of the first feature, and (iii) the set of amounts of sequence reads to determine whether the test subject has the cancer condition or the likelihood that test subject has the cancer condition.

Referring to block 310 of FIG. 2K, in some embodiments the plurality of sequence reads is evaluated to obtain an indication as to whether a sequence fragment signature associated with a first pathogen in the set of pathogens is present or absent. The plurality of sequence reads is also evaluated to obtain an indication as to whether a methylation signature associated with the first pathogen in the set of pathogens is present or absent. In such embodiments, the screening uses (i) the indication as to whether the signature fragment signature associated with the first pathogen is present or absent, (ii) an indication as to whether a methylation signature associated with the first pathogen is present or absent, (iii) the amount of the first feature, and (iv) the set of amounts of sequence reads to determine whether the test subject has the cancer condition or the likelihood that test subject has the cancer condition.

Referring to block 312 of FIG. 2K, in some embodiments the corresponding amount of the plurality of sequence reads that map to a sequence in a pathogen target reference for the respective pathogen is a percentage of the plurality of sequence reads from the test subject that map to a sequence in a pathogen target reference for the respective pathogen measured in the second biological sample.

Referring to block 314 of FIG. 2K, in some embodiments the determining a corresponding amount of the plurality of sequence reads that map to a sequence in a pathogen target reference for the corresponding pathogen comprises translating the plurality of sequence reads in a reading frame to form a plurality of translated sequence reads and comparing the plurality of translated sequence reads to a translation of the pathogen target reference.

Referring to block 316 of FIG. 2K, in some embodiments the determining a corresponding amount of the plurality of sequence reads that map to a sequence in a pathogen target reference for the corresponding pathogen comprises k-mer matching the plurality of sequence reads to the pathogen target reference in nucleic acid, ribonucleic acid or protein space.

Referring to block 318 of FIG. 2K, in some embodiments the test subject is human, and the second assay further comprises performing an end-point analysis of each respective amount of the plurality of sequence reads within the human genome.

Referring to block 320 of FIG. 2L, in some embodiments the plurality of sequence reads is evaluated to obtain an indication as to whether an APOBEC induced mutational signature associated with (e.g., the APOBEC induced mutational signature is related to the host viral immune response) a first pathogen in the set of pathogens is present or absent. In such embodiments, the screening uses (i) the indication as to whether the signature fragment signature associated with the first pathogen is present or absent, (ii) an indication as to whether a methylation signature associated with the first pathogen is present or absent, and (iii) the indication as to whether the APOBEC induced mutational signature associated with the first pathogen is present or absent to determine whether the test subject has the cancer condition or the likelihood that the test subject has the cancer condition. The APOBEC induced mutational signature, if present, will comprise an APOBEC/AID induced mutation in the host genome (see e.g., Wallace et al., 2018, PLoS Pathog 14(1) pp. e1006717, which is hereby incorporated by reference).

Referring to block 322 of FIG. 2L, in some embodiments the plurality of sequence reads is evaluated, via k-mer analysis, to obtain an indication as to whether APOBEC induced mutational signature associated with a first pathogen in the set of pathogens is present or absent. In such embodiments, the screening uses (i) the indication as to whether the signature fragment signature associated with the first pathogen is present or absent, (ii) an indication as to whether a methylation signature associated with the first pathogen is present or absent, and (iii) the indication as to whether the APOBEC induced mutational signature associated with the first pathogen is present or absent to determine whether the test subject has the cancer condition or the likelihood that the test subject has the cancer condition.

Referring to block 324 of FIG. 2L, in some embodiments the indication as to whether APOBEC induced mutational signature associated with a first pathogen in the set of pathogens is present or absent further includes a measure of enrichment of the APOBEC induced mutational signature. In such embodiments, the screening uses (i) the indication as to whether the signature fragment signature associated with the first pathogen is present or absent, (ii) an indication as to whether a methylation signature associated with the first pathogen is present or absent, and (iii) further includes a measure of enrichment of the APOBEC induced mutational signature to determine whether the test subject has the cancer condition or the likelihood that the test subject has the cancer condition.

Referring to block 326 of FIG. 2L, in some embodiments the first biological sample or a second biological sample from the test subject is analyzed for an expression of an APOBEC protein associated with a first pathogen in the set of pathogens. In such embodiments, the screening uses (i) the indication as to whether the signature fragment signature associated with a first pathogen is present or absent, (ii) the amount of the first feature, and (iii) the expression of the APOBEC protein associated with the first pathogen to determine whether the test subject has the cancer condition or the likelihood that the test subject has the cancer condition.

Referring to block 328 of FIG. 2M, in some embodiments a third assay is performed that comprises measuring an amount of an APOBEC induced mutational signature of the cell-free nucleic acid in the first biological sample. The screening uses (i) the indication as to whether the signature fragment signature associated with a first pathogen is present or absent, (ii) the amount of the first feature, and (iii) the amount of the APOBEC induced mutational signature to determine whether the test subject has the cancer condition or the likelihood that the test subject has the cancer condition.

Referring to block 330 of FIG. 2M, in some embodiments, performing the second assay further comprises measuring an amount of an APOBEC induced mutational signature of the cell-free nucleic acid in the second biological sample. The screening uses (i) the indication as to whether the signature fragment signature associated with a first pathogen is present or absent, (ii) the amount of the first feature, and (iii) the amount of the APOBEC induced mutational signature to determine whether the test subject has the cancer condition or the likelihood that the test subject has the cancer condition.

Referring to blocks 320-330, in some embodiments the APOBEC induced mutational signature is selected from either mutation signature type 2 or mutation signature type 13 as defined in Alexandrov et al., 2013, Nature 500(7463), pp. 415-421 and by Tate et al., 2019, Nuc. Acids Res. 47(D1), pp. D941-D947, which are hereby incorporated by reference. When either signature type 2 or type 13 is observed in the plurality of sequence reads obtained from the subject, it is determined that an APOBEC mutational process was present in the subject.

III. The presence of viral specific signatures for cancer detection. Methods of screening for a cancer condition in a test subject have been disclosed in Sections I and/or II above. The present section provides additional methods for screening for a cancer condition in a test subject. In this section any of the assays or methods described in Sections I and/or II is combined with another assay that measures a first feature in a test subject in order to screen for the cancer condition in a test subject. Moreover, the present section provides more details on the types of cancer conditions, types of sequence reads, and other experimental details that can be used in the methods of Sections I and/or II above.

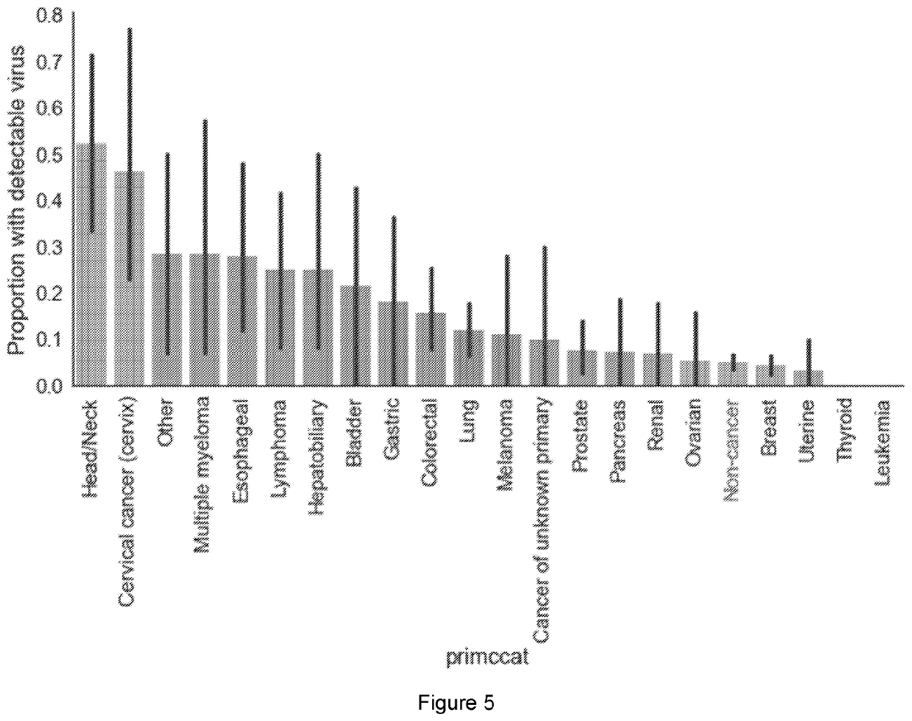

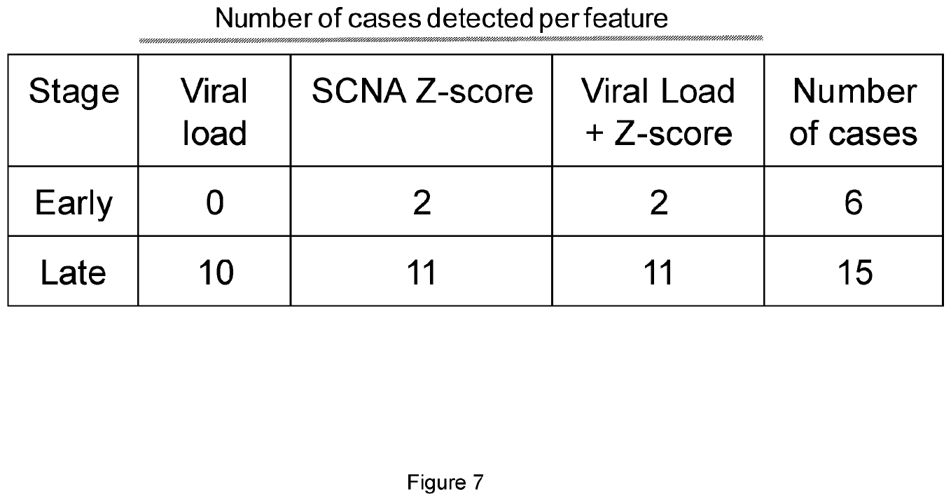

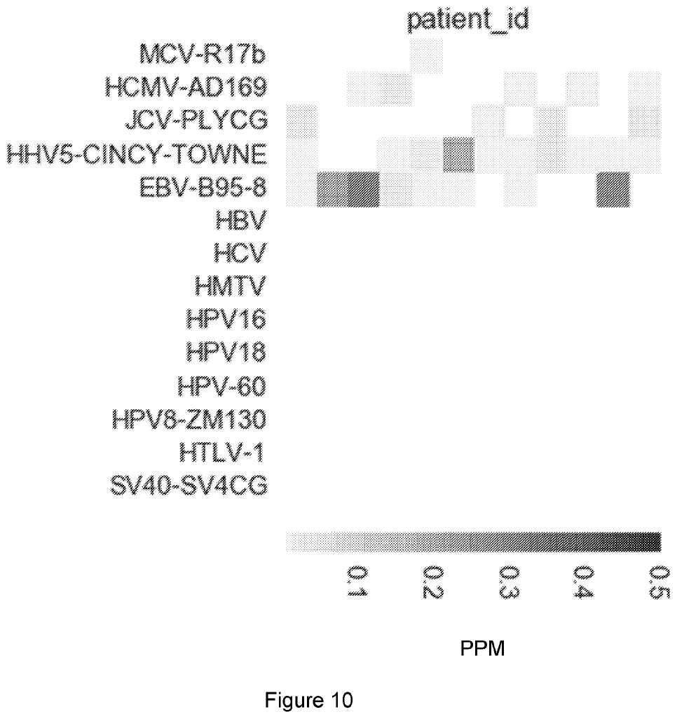

Another aspect of the present disclosure provides a method of screening for a cancer condition in a test subject. The method comprises obtaining a first biological sample from the test subject. The first biological sample comprises cell-free nucleic acid from the test subject and potentially cell-free nucleic acid from at least one pathogen in a set of pathogens. The method further comprises sequencing the cell-free nucleic acid in the first biological sample to generate a plurality of sequence reads from the test subject. The method further comprises evaluating the plurality of sequence reads to obtain an indication as to whether a sequence fragment signature associated with a respective pathogen in the set of pathogens is present or absent. As shown in FIG. 5, it is possible to detect viral fragments in a significant percentage of subjects with known cancer conditions (e.g., in particular viral signatures could be detected for patients with head and neck cancer or cervical cancer). FIG. 7 further illustrates that viral load can be correlated with stage (e.g., as stage increases, viral load increases). The data shown in FIG. 7 were obtained from patients with head and neck cancer. FIG. 10 further illustrates that, for subjects with breast cancer, the methods described herein are able to detect viral loads below levels that were detectable in previous studies (e.g., see, Tang et al., 2013, Nature Communications 4:2513). The method further comprises using the indication as to whether the fragment signature associated with the respective pathogen is present or absent to determine whether the test subject has the cancer condition or the likelihood that test subject has the cancer condition.

In some embodiments, evaluating the plurality of sequence reads further obtains an indication as to whether an APOBEC induced mutational signature associated with a first pathogen in the set of pathogens is present or absent. In such embodiments, the method further comprises using the indication as to whether the APOBEC induced mutational signature associated with the first pathogen is present or absent along with the indication as to whether the signature fragment signature associated with the respective pathogen is present or absent to determine whether the test subject has the cancer condition or the likelihood that the test subject has the cancer condition.

In some embodiments, the indication as to whether the APOBEC induced mutational signature associated with the first pathogen is present or absent further includes a measure of enrichment of the APOBEC induced mutational signature. In such embodiments, the method further comprises using the expression of the APOBEC protein along with the indication as to whether the signature fragment signature associated with the respective pathogen is present or absent to determine whether the test subject has the cancer condition or the likelihood that the test subject has the cancer condition.

In some embodiments, the first biological sample or a second biological sample from the test subject is analyzed for an expression of an APOBEC protein associated with a first pathogen in the set of pathogens. In such embodiments, the method further comprises using the expression of the APOBEC protein along with the indication as to whether the signature fragment signature associated with the respective pathogen is present or absent to determine whether the test subject has the cancer condition or the likelihood that the test subject has the cancer condition.

In some embodiments, the method further comprises using the amount of the APOBEC induced mutational signature and the set of amounts of sequence reads to determine whether the test subject has the cancer condition or the likelihood that the test subject has the cancer condition.

In some embodiments, a second biological sample is obtained from the test subject. The second biological sample comprises cell-free nucleic acid from the test subject and potentially cell-free nucleic acid from a first pathogen in the set of pathogens. An assay is performed that comprises measuring an amount of an APOBEC induced mutational signature of the cell-free nucleic acid in the second biological sample. In such embodiments, the method further comprises using the amount of the APOBEC induced mutational signature and the set of amounts of sequence reads to determine whether the test subject has the cancer condition or the likelihood that the test subject has the cancer condition.

IV. The presence of a methylation signature detection of a cancer condition. Another aspect of the present disclosure provides a method of screening for a cancer condition in a test subject in which a biological sample is obtained from the test subject. The biological sample comprises cell-free nucleic acid from the test subject and potentially cell-free nucleic acid from at least one pathogen in a set of pathogens. The method further comprises sequencing the cell-free nucleic acid in the biological sample to generate a plurality of sequence reads from the test subject. The method further comprises evaluating the plurality of sequence reads to obtain an indication as to whether a methylation signature associated with a respective pathogen in the set of pathogens is present or absent. The method further comprises using the indication as to whether the methylation signature associated with the respective pathogen is present or absent to determine whether the test subject has the cancer condition or the likelihood that test subject has the cancer condition.

In some embodiments, evaluating the plurality of sequence reads further obtains an indication as to whether an APOBEC induced mutational signature associated with a first pathogen in the set of pathogens is present or absent. In such embodiments, the method further comprises the using the indication as to whether the APOBEC induced mutational signature associated with the first pathogen is present or absent along with the indication as to whether the methylation signature associated with the respective pathogen is present or absent to determine whether the test subject has the cancer condition or the likelihood that the test subject has the cancer condition.

In some embodiments, the indication as to whether the APOBEC induced mutational signature associated with the first pathogen is present or absent further includes a measure of enrichment of the APOBEC induced mutational signature. In such embodiments, the method further comprises using the measure of enrichment of the APOBEC induced mutational signature along with the indication as to whether the methylation signature associated with the respective pathogen is present or absent to determine whether the test subject has the cancer condition or the likelihood that the test subject has the cancer condition.

In some embodiments, the first biological sample or a second biological sample is analyzed from the test subject for an expression of an APOBEC protein associated with a first pathogen in the set of pathogens. In such embodiments, the method further comprises using the expression of the APOBEC protein along with the indication as to whether the methylation signature associated with the respective pathogen is present or absent to determine whether the test subject has the cancer condition or the likelihood that the test subject has the cancer condition.

In some embodiments, an assay is performed that comprises measuring an amount of an APOBEC induced mutational signature of the cell-free nucleic acid in the first biological sample. In such embodiments, the method further comprises using the indication as to whether the APOBEC induced mutational signature associated with the first pathogen is present or absent along with the indication as to whether the methylation signature associated with the respective pathogen is present or absent to determine whether the test subject has the cancer condition or the likelihood that the test subject has the cancer condition.

In some embodiments, a second biological sample is obtained from the test subject. The second biological sample comprises cell-free nucleic acid from the test subject and potentially cell-free nucleic acid from a first pathogen in the set of pathogens. An assay is performed that comprises measuring an amount of an APOBEC induced mutational signature of the cell-free nucleic acid in the second biological sample. In such embodiments, the method further comprises using the indication as to whether the APOBEC induced mutational signature associated with the first pathogen is present or absent along with the indication as to whether the methylation signature associated with the respective pathogen is present or absent to determine whether the test subject has the cancer condition or the likelihood that the test subject has the cancer condition. In some embodiments, the APOBEC protein is APOBEC1, APOBEC2, APOBEC3A, APOBEC3B, APOBEC3C, APOBEC3D, APOBEC3F, APOBEC3G, APOBEC3H, or APOBEC4.

V. The presence of a pathogen specific signature and a methylation signature for detection of a cancer condition. Another aspect of the present disclosure provides a method of screening for a cancer condition in a test subject in which a first biological sample is obtained from the test subject. The first biological sample comprises cell-free nucleic acid from the test subject and potentially cell-free nucleic acid from at least one pathogen in a set of pathogens. The method further comprises sequencing the cell-free nucleic acid in the first biological sample to generate a plurality of sequence reads from the test subject. The method further comprises evaluating the plurality of sequence reads to obtain an indication as to whether a sequence fragment signature associated with a respective pathogen in the set of pathogens is present or absent. The method further comprises evaluating the plurality of sequence reads to obtain an indication as to whether a methylation signature associated with a respective pathogen in the set of pathogens is present or absent. The method further comprises using the indication as to whether the signature fragment signature associated with a respective pathogen is present or absent and the indication as to whether the methylation signature associated with a respective pathogen is present or absent to determine whether the test subject has the cancer condition or the likelihood that test subject has the cancer condition.

In some embodiments, the plurality of sequence reads is evaluated to obtain an indication as to whether an APOBEC induced mutational signature associated with a first pathogen in the set of pathogens is present or absent. In some embodiments, the method further comprises using (i) the indication as to whether the signature fragment signature associated with a respective pathogen is present or absent, (ii) the indication as to whether the methylation signature associated with a respective pathogen is present or absent, and (iii) the indication as to whether an APOBEC induced mutational signature associated with a first pathogen in the set of pathogens to determine whether the test subject has the cancer condition or the likelihood that the test subject has the cancer condition.

In some embodiments, the method further comprises using (i) the indication as to whether the signature fragment signature associated with a respective pathogen is present or absent, (ii) the indication as to whether the methylation signature associated with a respective pathogen is present or absent, and (iii) the indication as to whether an APOBEC induced mutational signature associated with a first pathogen in the set of pathogens to determine whether the test subject has the cancer condition or the likelihood that the test subject has the cancer condition.

In some embodiments, the indication as to whether the APOBEC induced mutational signature associated with the first pathogen is present or absent further includes a measure of enrichment of the APOBEC induced mutational signature. In some embodiments, the method further comprises using (i) the indication as to whether the signature fragment signature associated with a respective pathogen is present or absent, (ii) the indication as to whether the methylation signature associated with a respective pathogen is present or absent, and (iii) the measure of enrichment of the APOBEC induced mutational signature to determine whether the test subject has the cancer condition or the likelihood that the test subject has the cancer condition.

In some embodiments, the method further comprises analyzing the first biological sample or a second biological sample from the test subject for an expression of an APOBEC protein associated with a first pathogen in the set of pathogens. In some embodiments, the method further comprises using (i) the indication as to whether the signature fragment signature associated with a respective pathogen is present or absent, (ii) the indication as to whether the methylation signature associated with a respective pathogen is present or absent, and (iii) the expression of an APOBEC protein associated with a first pathogen in the set of pathogens to determine whether the test subject has the cancer condition or the likelihood that the test subject has the cancer condition.

In some embodiments, the method further comprises performing an assay comprising measuring an amount of an APOBEC induced mutational signature of the cell-free nucleic acid in the first biological sample. In some embodiments, the method further comprises using (i) the indication as to whether the signature fragment signature associated with a respective pathogen is present or absent, (ii) the indication as to whether the methylation signature associated with a respective pathogen is present or absent, and (iii) the amount of the APOBEC induced mutational signature and the set of amounts of sequence reads to determine whether the test subject has the cancer condition or the likelihood that the test subject has the cancer condition.

In some embodiments, the method continues by performing an assay that comprises measuring an amount of an APOBEC induced mutational signature of the cell-free nucleic acid in the second biological sample. In such embodiments, the method further comprises using (i) the indication as to whether the signature fragment signature associated with a respective pathogen is present or absent, (ii) the indication as to whether the methylation signature associated with a respective pathogen is present or absent, and (iii) the amount of the APOBEC induced mutational signature and the set of amounts of sequence reads to determine whether the test subject has the cancer condition or the likelihood that the test subject has the cancer condition.

VI. Pathogen panel for cancer screening. Another aspect of the present disclosure provides a pathogen panel for screening for a test subject to determine a likelihood or indication that the subject has a cancer condition, the viral panel comprising a first sequence fragment and a second sequence fragment. The first sequence fragment and the second sequence fragment are each independently a fragment of the genome of a corresponding parasite in a set of parasites consisting of human herpes virus 5 CINCY-TOWNE (HHV5-CINCY-TOWNE) virus, Epstein-Barr B95-8 (EBV-B95-8 virus), molluscum contagiosum virus R17b (MCV-R17b) virus, human papillomavirus 16 (HPV16) virus, human cytomegalovirus AD169 (HCMV-AD169) virus, hepatitis B virus (HBV) virus, hepatitis B virus 18 (HPV18) virus, hepatitis C virus (HCV) virus, human papillomavirus 8-ZM130 (HPV8-ZM130) virus, and John Cunningham virus PLYCG (JCV-PLYCG) virus. The first sequence fragment is a fragment of a parasite other than that of the first sequence fragment.

In some embodiments, the first sequence fragment encodes at least one hundred bases of the genome of the corresponding parasite. In some embodiments, the viral panel includes a sequence fragment for at least four different parasites in the set of parasites. In some embodiments, the viral panel includes a sequence fragment for at least five different parasites in the set of parasites.

In some embodiments, the pathogen panel includes a sequence fragment for at least eight different parasites in the set of parasites. In some embodiments, the pathogen panel includes at least fifty sequence fragments from parasites in the set of parasites.

In some embodiments, the first sequence fragment encodes a portion of a protein encoded by the genome of the corresponding parasite. In some embodiments, the first sequence fragment encodes a methylation pattern of a portion of the genome of the corresponding parasite.

VII. The presence of a pathogen specific signature and APOBEC induced mutational signature for detection of a cancer condition. Another aspect of the present disclosure uses a measure of enrichment of APOBEC induced mutational signature as a basis for screening for cancer. In such embodiments, screening for a cancer condition or a likelihood of having the first condition in a test subject of a species comprises obtaining a first biological sample from the test subject. The first biological sample comprises cell-free nucleic acid from the test subject. In the method, cell-free nucleic acid in the first biological sample is sequenced (e.g., by whole genome sequencing, targeted panel sequencing—methylation or non-methylation related, or whole genome bisulfite sequencing) to generate a plurality of sequence reads from the test subject. The plurality of sequence reads is then analyzed for a measure of enrichment of a first APOBEC induced mutational signature. The measure of enrichment of the first APOBEC induced mutational signature is then used to determine whether the test subject has the cancer condition or the likelihood of having the cancer condition.

In some embodiments, the analyzing comprises k-mer analysis of the plurality of sequence reads to determine the measure of enrichment of the first APOBEC induced mutational signature. In some embodiments, the analyzing comprises a sequence alignment between (i) one or more sequence reads in the plurality of sequence reads and (ii) the first APOBEC induced mutational signature, thereby obtaining the measure of enrichment of the first APOBEC induced mutational signature.

In some embodiments, the measure of enrichment of the first APOBEC induced mutational signature is in the form of a p-value against an amount of the first APOBEC induced mutational signature across a cohort of the species that does not have the cancer, the test subject is deemed to have the cancer condition or the likelihood of having the cancer condition when the p-value is in a threshold range, and the test subject is deemed to not have the cancer condition or the likelihood of having the cancer condition when the p-value is not in the threshold range. In some such embodiments, the threshold range is less than or equal to 0.00001, less than or equal to 0.0001, less than or equal to 0.001, less than or equal to 0.002, less than or equal to 0.003, less than or equal to 0.004, less than or equal to 0.005, less than or equal to 0.01, less than or equal to 0.02, less than or equal to 0.03, less than or equal to 0.04, or less than or equal to 0.05.

In some embodiments, the first APOBEC induced mutational signature is associated with a pathogen. That is, the presence of the APOBEC induced mutational signature, or the measure of APOBEC induced mutational signature in the sequences reads of the subject indicates that a particular pathogen is present in the subject.

In some embodiments, the above-described analyzing further comprises using k-mer analysis of the plurality of sequence reads to determine an amount of the plurality of sequence reads that map to a reference genome of the pathogen and the using also uses the amount of the plurality of sequence reads that map to the reference genome of the pathogen to determine whether the test subject has the cancer condition or the likelihood of having the cancer condition. In some embodiments, the k-mer analysis further comprises dividing each sequence read in the plurality of sequence reads into a plurality of substrings of a predetermined size, thereby obtaining a set of substrings for each respective sequence read in the plurality of sequence reads for the test subject, and the analyzing compares each substring across all or a portion of the reference genome of the pathogen. In some such embodiments, the predetermined size is selected from the set of 1-10, 5-10, 10-80, 20-35, or 20-25 nucleic acids.

In some embodiments, the pathogen is Epstein-Barr virus (EBV), human cytomegalovirus (HCMV), hepatitis B virus (HBV), hepatitis C virus (HCV), human herpes virus (HHV), human mammary tumor virus (HMTV), human papillomavirus 16 (HPV16), human papillomavirus 18 (HPV18), human papillomavirus 60 (HPV-60), human papillomavirus ZM130 (HPV8-ZM130), human T-cell leukemia virus type 1 (HTLV-1), John Cunningham virus (JCV), molluscum contagiosum virus (MCV), or simian vacuolating virus 40 (SV40).

In some embodiments, the method further comprises analyzing the first biological sample or another biological sample from the test subject for an expression of an APOBEC protein associated with the cancer condition, and the using the measure of enrichment of the first APOBEC induced mutational signature further comprises using the expression of the APOBEC protein to determine whether the test subject has the cancer condition or the likelihood of having the cancer condition. In some embodiments, the species is human.

In some embodiments, the cancer condition is breast, lung, prostate, colorectal, renal, uterine, pancreatic, esophagus, lymphoma, head/neck, ovarian, a hepatobiliary, melanoma, cervical, multiple myeloma, leukemia, thyroid, bladder, gastric, or a combination thereof. In some embodiments, the cancer condition is a predetermined stage (e.g., stage I, stage II, stage III, or stage IV) thereof. In some embodiments, the first biological sample comprises blood, whole blood, plasma, serum, urine, cerebrospinal fluid, fecal, saliva, sweat, tears, pleural fluid, pericardial fluid, or peritoneal fluid or any combination thereof.

In some embodiments, the method further comprises providing a therapeutic intervention or imaging of the test subject based on a determination that the test subject has the cancer condition or the likelihood of having the cancer condition.

In some embodiments, the analyzing further comprises analyzing for a measure of enrichment of a second APOBEC induced mutational signature and the using further comprises using the measure of enrichment of the second APOBEC induced mutational signature to determine whether the test subject has the cancer condition or the likelihood of having the cancer condition.

In some embodiments, the measure of enrichment of the first APOBEC induced mutational signature satisfies a predetermined enrichment threshold, the test subject is deemed to have the cancer condition or the likelihood of having the cancer condition, and when the measure of enrichment of the first APOBEC induced mutational signature fails to satisfy the predetermined enrichment threshold, the test subject is deemed to not have the cancer condition or the likelihood of having the cancer condition.

In some embodiments, the measure of enrichment of the first APOBEC induced mutational signature is determined by comparing an expected amount of sequence reads for the first APOBEC induced mutational signature to the enrichment of the first APOBEC induced mutational signature. In some such embodiments, the expected amount of sequence reads for the first APOBEC signature is about 5, 7, 10, 12 or 20 sequence reads of the first APOBEC signature.

Another aspect of the present disclosure provides a computer system for screening for a cancer condition or a likelihood of having the first condition in a test subject of a species. The computer system comprises one or more processors, a memory, and one or more programs. The one or more programs are stored in the memory and are configured to be executed by the one or more processors. The one or more programs including instructions for analyzing a plurality of sequence reads for a measure of enrichment of a first APOBEC induced mutational signature. The plurality of sequence reads is obtained from a first biological sample from the test subject. The first biological sample comprises cell-free nucleic acid from the test subject. The one or more programs further includes instructions for sequencing the cell-free nucleic acid in the first biological sample to generate a plurality of sequence reads from the test subject. The one or more programs further includes instructions for using the measure of enrichment of the first APOBEC induced mutational signature to determine whether the test subject has the cancer condition or the likelihood of having the cancer condition.

Still another aspect of the present disclosure provides a non-transitory computer readable storage medium and one or more computer programs embedded therein for screening for a cancer condition or a likelihood of having the first condition in a test subject of a species. The one or more computer programs comprise instructions that, when executed by a computer system, cause the computer system to perform a method comprising analyzing a plurality of sequence reads for a measure of enrichment of a first APOBEC induced mutational signature. The plurality of sequence reads is obtained from a first biological sample of the test subject, where the first biological sample comprises cell-free nucleic acid from the test subject. The one or more computer programs further comprise instructions for sequencing the cell-free nucleic acid in the first biological sample to generate a plurality of sequence reads from the test subject. The one or more computer programs comprise instructions using the measure of enrichment of the first APOBEC induced mutational signature to determine whether the test subject has the cancer condition or the likelihood of having the cancer condition.

Another aspect of the present disclosure provides a method for screening for a cancer condition or a likelihood of having the first condition in a test subject of a species. The method comprises obtaining a first biological sample from the test subject, where the first biological sample comprises cell-free nucleic acid from the test subject. The cell-free nucleic acid in the first biological sample are then sequenced (e.g., by whole genome sequencing, targeted panel sequencing: methylation or non-methylation related, or whole genome bisulfite sequencing, etc.) to generate a plurality of sequence reads from the test subject. Then, k-mer analysis is used to determine an amount of the plurality of sequence reads that map to a pathogen target reference. The amount of sequence reads is used to determine whether the test subject has the cancer condition or the likelihood of having the cancer condition. In some embodiments, the pathogen target reference is associated with a first pathogen. In some embodiments, this first pathogen is associated with a first viral infection type. In some embodiments, the test subject has the first viral infection type.

In some embodiments, the pathogen target reference consists of a panel of target sequences that collectively represent a subset of a pathogen reference genome for the first pathogen and the using limits, for the pathogen, the mapping of each sequence read in the plurality of sequence reads to the corresponding targeted panel of sequences from the pathogen reference genome.

In some embodiments, the pathogen target reference for the first pathogen is a reference genome of the first pathogen or a portion thereof, and the using compares, for the first pathogen, a methylation pattern of one or more sequence reads in the plurality of sequence reads to a methylation pattern across all or a portion of the reference genome of the first pathogen.

In some embodiments, the k-mer analysis further comprises dividing each sequence read in the plurality of sequence reads into a plurality of sub strings of a predetermined size, thereby obtaining a set of substrings for the test subject, and the using compares each substring in the plurality of substrings across all or a portion of the reference genome of the first pathogen. In some embodiments the predetermined size is selected from the set of 1-10, 5-10, 10-80, 20-35, or 20-25 nucleic acids.

In some embodiments, the cancer condition is breast, lung, prostate, colorectal, renal, uterine, pancreatic, cancer of the esophagus, lymphoma, head/neck, ovarian, hepatobiliary, melanoma, cervical, multiple myeloma, leukemia, thyroid, bladder, gastric, or a combination thereof or a predetermined stage (e.g., stage I, stage II, stage III, or stage IV) thereof.

In some embodiments, the k-mer analysis comprises translating the plurality of sequence reads from the test subject in a reading frame to form a plurality of translated sequence reads and comparing the plurality of translated sequence reads to a translation of each sequence in the pathogen target reference. In some embodiments, the k-mer analysis compares the plurality of sequence reads from the test subject to the pathogen reference genome in nucleic acid, ribonucleic acid, or protein space.

In some embodiments, the method further comprises analyzing the first biological sample or another biological sample from the test subject for an expression of an APOBEC protein associated with the cancer condition, and the using the amount of sequence reads further comprises using the expression of the APOBEC protein in conjunction with the amount of sequence reads to determine whether the test subject has the cancer condition or the likelihood of having the cancer condition.

In some embodiments, the amount of sequence reads in the plurality of sequence reads is in the form of a p-value against an amount of sequence reads that map to the pathogen target reference across a cohort of the species that does not have the cancer, the test subject is deemed to have the cancer condition or the likelihood of having the cancer condition when the p-value is in a threshold range, and the test subject is deemed to not have the cancer condition or the likelihood of having the cancer condition when the p-value is not in the threshold range.

In some embodiments, the threshold range is less than or equal to 0.00001, less than or equal to 0.0001, less than or equal to 0.001, less than or equal to 0.002, less than or equal to 0.003, less than or equal to 0.004, less than or equal to 0.005, less than or equal to 0.01, less than or equal to 0.02, less than or equal to 0.03, less than or equal to 0.04, or less than or equal to 0.05.

In some embodiments, the method further comprises providing a therapeutic intervention or imaging of the test subject based on the determination of whether the test subject has the cancer condition or the likelihood that the test subject has the cancer condition.

Another aspect of the present disclosure provides a computer system for screening for a cancer condition or a likelihood of having the first condition in a test subject of a species. The computer system comprises one or more processors, a memory, and one or more programs. The one or more programs are stored in the memory and are configured to be executed by the one or more processors. The one or more programs include instructions for using k-mer analysis to determine an amount of the plurality of sequence reads that map to a pathogen target reference where the plurality of sequence reads is obtained from a first biological sample from the test subject, and where the first biological sample comprises cell-free nucleic acid from the test subject and using the amount of sequence reads to determine whether the test subject has the cancer condition or the likelihood of having the cancer condition.

Still another aspect of the present disclosure provides a non-transitory computer readable storage medium and one or more computer programs embedded therein for screening for a cancer condition or a likelihood of having the first condition in a test subject of a species. The one or more computer programs comprise instructions that, when executed by a computer system, cause the computer system to perform a method comprising using k-mer analysis to determine an amount of the plurality of sequence reads that map to a pathogen target reference, where the plurality of sequence reads is obtained from a first biological sample from the test subject, and where the first biological sample comprises cell-free nucleic acid from the test subject. The one or more computer programs further comprise instructions for using the amount of sequence reads to determine whether the test subject has the cancer condition or the likelihood of having the cancer condition.

Providing classification method based on a longitudinal study. Still another aspect of the present disclosure is directed to developing a classifier using a longitudinal study of reference subjects. In accordance with this aspect of the present disclosure, a classification method is provided that comprises, at a computer system having one or more processors, and memory storing one or more programs for execution by the one or more processors, for each respective reference subject in a cohort of subjects of a species, where a first portion of the cohort of subjects have a cancer condition and a second portion of the cohort of subjects do not have the cancer condition, performing a first procedure. The first procedure comprises obtaining a corresponding first biological sample from the respective reference cancer subject representative, where the corresponding first biological comprises cell-free nucleic acid, and sequencing the cell-free nucleic acid in the corresponding first biological sample to generate a corresponding first plurality of sequence reads. The one or more programs further comprise instructions for analyzing the corresponding first plurality of sequence reads of each respective reference cancer subject in the cohort for a measure of enrichment of an APOBEC induced mutational signature.

The above is repeated for one or more time points across a predetermined time period, thereby obtaining a corresponding longitudinal set of measures of APOBEC signature enrichment for each respective reference subject in the cohort. The corresponding longitudinal set of measures of APOBEC signature enrichment for each respective subject in the cohort along with a first label of whether the corresponding longitudinal set of measures of APOBEC signature enrichment is from a cohort subject that has the cancer condition or does not have the cancer condition is applied to an untrained classifier thereby obtaining a trained classifier that is configured to determine whether a test subject of the species has the cancer condition based on a measure of APOBEC signature enrichment of the test subject.

In some such embodiments, a third portion of the cohort of subjects have a first viral condition and a fourth portion of the cohort of subjects do not have the viral condition, and the applying further applies a second label of whether the corresponding longitudinal set of measures of APOBEC signature enrichment is from a cohort subject that has the first viral condition or does not have the first viral condition, and the trained classifier that is configured to determine whether the test subject of the species has the cancer condition makes the determination based on the measure of APOBEC signature enrichment of the test subject and an indication of whether the test subject has the viral condition. In some embodiments, the third portion of the cohort of subjects includes subjects in the first portion of subjects or the second portion of subjects, and the fourth portion of the cohort of subjects includes subjects in the first portion of subjects or the second portion of subjects.

In some embodiments, a fifth portion of the cohort of subjects have an overexpression of an APOBEC protein associated with the cancer condition and a sixth portion of the cohort of subjects do not have an overexpression of the APOBEC protein associated with the cancer condition, and the applying further applies an amount of expression of the APOBEC protein in each biological sample from each respective cohort subject, and the trained classifier that is configured to determine whether the test subject has the cancer condition makes the determination based on a measure of APOBEC signature enrichment of the test subject, an indication of whether the test subject has the viral condition, and an amount of expression of the APOBEC protein in a biological sample from the test subject. In some embodiments, the fifth portion of the cohort of subjects includes subjects in the first or second portion of subjects, and the sixth portion of the cohort of subjects includes subjects in the first or second portion of subjects. In some such embodiments, the fifth portion of the cohort of subjects includes subjects in the first or second portion of subjects, and the sixth portion of the cohort of subjects includes subjects in the or second first portion of subjects.

In some embodiments, the classification method further comprises obtaining a test biological sample from a test subject, where the test biological sample comprises cell-free nucleic acid, sequencing the cell-free nucleic acid in the test biological sample to generate a plurality of test sequence reads and analyzing the plurality of test sequence reads for a test measure of enrichment of an APOBEC induce mutational signature and applying the test measure of APOBEC signature enrichment to the trained classifier, thereby obtaining a classifier result indicating whether the test subject has the cancer condition.

In some such embodiments, the sequencing is performed by whole genome sequencing, targeted panel sequencing: methylation or non-methylation related, or whole genome bisulfite sequencing. In some embodiments, the analyzing the first plurality of sequence reads for enrichment of the APOBEC induced mutational signature comprises aligning each sequence read in the plurality of sequence reads to a lookup table of APOBEC induced mutational signatures in order to determine whether the sequence read contains all or a portion of an APOBEC induced mutational signature.

In some embodiments, the analyzing the first plurality of sequence reads for enrichment of the APOBEC induced mutational signature comprises performing k-mer analysis on each respective sequence read in the plurality of sequence reads to determine whether the respective sequence read contain all or a portion of the APOBEC induced mutational signature.

In some embodiments, the enrichment of the first APOBEC induced mutational signature is determined by comparing an expected amount of sequence reads for the APOBEC induced mutational signature to the measure of enrichment of the first APOBEC induced mutational signature.

In some embodiments, the APOBEC induced mutational signature is either APOBEC signature type 2 or APOBEC signature type 13. In some embodiments, the trained classifier is a binomial classifier. In some embodiments, the trained classifier is a logistic regression, neural network, support vector machine, or decision tree algorithm. In some embodiments, the classifier is a multinomial classifier that determines whether the subject has a first or second cancer condition.

In some embodiments, the trained classifier is a logistic regression algorithm that provides a likelihood that the test subject has or does not have the cancer condition. In some embodiments, the logistic regression provides a binary assessment of whether the test subject has or does not have the cancer condition. In some embodiments, the predetermined time period comprises at least 1, 2, 3, 4, 5, 6, or 12 months and the one or more time points comprises at least 2, 4, 6, 8, or 10 time points distributed throughout the predetermined time period.

In some embodiments, the first viral condition is Epstein-Barr virus (EBV), human cytomegalovirus (HCMV), hepatitis B virus (HBV), hepatitis C virus (HCV), human herpes virus (HHV), human mammary tumor virus (HMTV), human papillomavirus 16 (HPV16), human papillomavirus 18 (HPV18), human papillomavirus 60 (HPV-60), human papillomavirus ZM130 (HPV8-ZM130), human T-cell leukemia virus type 1 (HTLV-1), John Cunningham virus (JCV), molluscum contagiosum virus (MCV), or simian vacuolating virus 40 (SV40).

In some embodiments, the cohort of subjects of the species comprises at least 20, 50, 100, 200 or 500 subjects. In some embodiments, the method further comprises providing a therapeutic intervention or imaging of the test subject based on the determination of whether the test subject has the cancer condition.

Another aspect of the present disclosure provides a computer system for classification. The computer system comprises one or more processors, a memory, and one or more programs. The one or more programs are stored in the memory and are configured to be executed by the one or more processors. The one or more programs include instructions to perform any and all of the embodiments and methods described above. Another aspect of the present disclosure provides a non-transitory computer readable storage medium and one or more computer programs embedded therein for classification. The one or more computer programs comprise instructions that, when executed by a computer system, cause the computer system to perform any and all of the embodiments and methods described above.

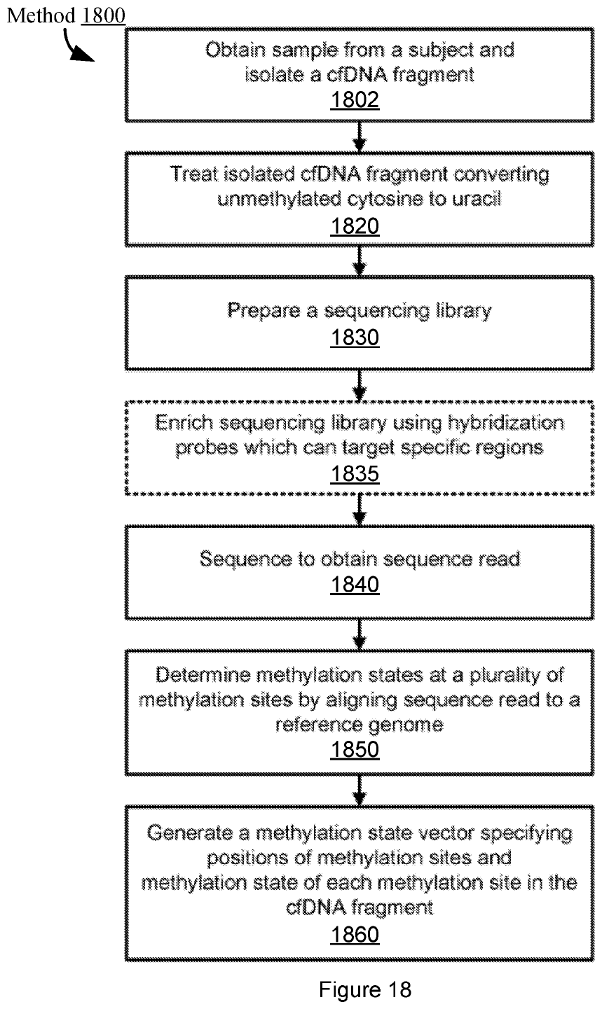

EXAMPLE 1—Generation of Methylation State Vector. FIG. 18 is a flowchart

describing a process 1800 of sequencing a fragment of cfDNA to obtain a methylation state vector, according to an embodiment in accordance with the present disclosure. Referring to step 1802, the cfDNA fragments are obtained from the biological sample (e.g., as discussed above in conjunction with FIG. 2). Referring to step 1820, the cfDNA fragments are treated to convert unmethylated cytosines to uracils. In one embodiment, the DNA is subjected to a bisulfite treatment that converts the unmethylated cytosines of the fragment of cfDNA to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, Calif.)) is used for the bisulfite conversion in some embodiments. In other embodiments, the conversion of unmethylated cytosines to uracils is accomplished using an enzymatic reaction. For example, the conversion can use a commercially available kit for conversion of unmethylated cytosines to uracils, such as APOBEC-Seq (NEBiolabs, Ipswich, Mass.).

From the converted cfDNA fragments, a sequencing library is prepared (step 1830). Optionally, the sequencing library is enriched 1835 for cfDNA fragments, or genomic regions, that are informative for cancer status using a plurality of hybridization probes. The hybridization probes are short oligonucleotides capable of hybridizing to particularly specified cfDNA fragments, or targeted regions, and enriching for those fragments or regions for subsequent sequencing and analysis. Hybridization probes may be used to perform a targeted, high-depth analysis of a set of specified CpG sites of interest to the researcher. Once prepared, the sequencing library or a portion thereof can be sequenced to obtain a plurality of sequence reads (1840). The sequence reads may be in a computer-readable, digital format for processing and interpretation by computer software

From the sequence reads, a location and methylation state for each of CpG site is determined based on alignment of the sequence reads to a reference genome (1850). A methylation state vector for each fragment specifying a location of the fragment in the reference genome (e.g., as specified by the position of the first CpG site in each fragment, or another similar metric), a number of CpG sites in the fragment, and the methylation state of each CpG site in the fragment (1860).

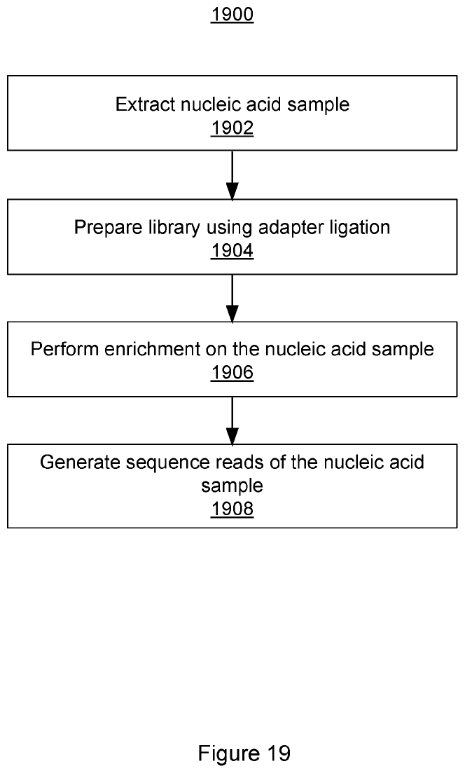

EXAMPLE 2—Obtaining a Plurality of Sequence reads. FIG. 19 is flowchart of a method 1900 for preparing a nucleic acid sample for sequencing according to one embodiment. The method 1900 includes, but is not limited to, the following steps. For example, any step of the method 1900 may comprise a quantitation sub-step for quality control or other laboratory assay procedures known to one skilled in the art.

In block 1902, a nucleic acid sample (DNA or RNA) is extracted from a subject. The sample may be any subset of the human genome, including the whole genome. The sample may be extracted from a subject known to have or suspected of having cancer. The sample may include blood, plasma, serum, urine, fecal, saliva, other types of bodily fluids, or any combination thereof. In some embodiments, methods for drawing a blood sample (e.g., syringe or finger prick) may be less invasive than procedures for obtaining a tissue biopsy, which may require surgery. The extracted sample may comprise cfDNA and/or ctDNA. For healthy individuals, the human body may naturally clear out cfDNA and other cellular debris. If a subject has a cancer or disease, ctDNA in an extracted sample may be present at a detectable level for diagnosis.

In block 1904, a sequencing library is prepared. During library preparation, unique molecular identifiers (UMI) are added to the nucleic acid molecules (e.g., DNA molecules) through adapter ligation. The UMIs are short nucleic acid sequences (e.g., 4-10 base pairs) that are added to ends of DNA fragments during adapter ligation. In some embodiments, UMIs are degenerate base pairs that serve as a unique tag that can be used to identify sequence reads originating from a specific DNA fragment. During PCR amplification following adapter ligation, the UMIs are replicated along with the attached DNA fragment. This provides a way to identify sequence reads that came from the same original fragment in downstream analysis.

In block 1906, targeted DNA sequences are enriched from the library. During enrichment, hybridization probes (also referred to herein as “probes”) are used to target, and pull down, nucleic acid fragments informative for the presence or absence of cancer (or disease), cancer status, or a cancer classification (e.g., cancer type or tissue of origin). For a given workflow, the probes may be designed to anneal (or hybridize) to a target (complementary) strand of DNA. The target strand may be the “positive” strand (e.g., the strand transcribed into mRNA, and subsequently translated into a protein) or the complementary “negative” strand. The probes may range in length from 10s, 100s, or 1000s of base pairs. In one embodiment, the probes are designed based on a gene panel to analyze particular mutations or target regions of the genome (e.g., of the human or another organism) that are suspected to correspond to certain cancers or other types of diseases. Moreover, the probes may cover overlapping portions of a target region.

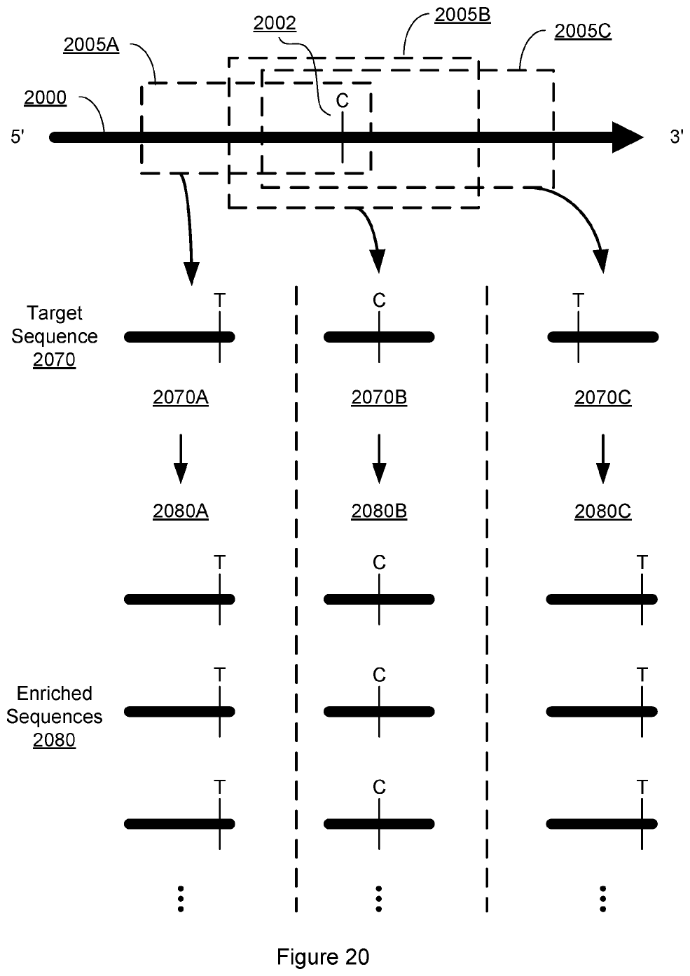

FIG. 20 is a graphical representation of the process for obtaining sequence reads according to one embodiment. FIG. 20 depicts one example of a nucleic acid segment 2000 from the sample. Here, the nucleic acid segment 2000 can be a single-stranded nucleic acid segment, such as a single stranded. In some embodiments, the nucleic acid segment 2000 is a double-stranded cfDNA segment. The illustrated example depicts three regions 2005A, 2005B, and 2005C of the nucleic acid segment that can be targeted by different probes. Specifically, each of the three regions 2005A, 2005B, and 2005C includes an overlapping position on the nucleic acid segment 2000. An example overlapping position is depicted in FIG. 20 as the cytosine (“C”) nucleotide base 2002. The cytosine nucleotide base 2002 is located near a first edge of region 2005A, at the center of region 2005B, and near a second edge of region 2005C.

In some embodiments, one or more (or all) of the probes are designed based on a gene panel to analyze particular mutations or target regions of the genome (e.g., of the human or another organism) that are suspected to correspond to certain cancers or other types of diseases. By using a targeted gene panel rather than sequencing all expressed genes of a genome, also known as “whole exome sequencing,” the method 2000 may be used to increase sequencing depth of the target regions, where depth refers to the count of the number of times a given target sequence within the sample has been sequenced. Increasing sequencing depth reduces required input amounts of the nucleic acid sample.

Hybridization of the nucleic acid sample 2000 using one or more probes results in an understanding of a target sequence 2070. As shown in FIG. 20, the target sequence 2070 is the nucleotide base sequence of the region 2005 that is targeted by a hybridization probe. The target sequence 2070 can also be referred to as a hybridized nucleic acid fragment. For example, target sequence 2070A corresponds to region 2005A targeted by a first hybridization probe, target sequence 2070B corresponds to region 2005B targeted by a second hybridization probe, and target sequence 2070C corresponds to region 2005C targeted by a third hybridization probe. Given that the cytosine nucleotide base 2002 is located at different locations within each region 2005A-C targeted by a hybridization probe, each target sequence 2070 includes a nucleotide base that corresponds to the cytosine nucleotide base 2002 at a particular location on the target sequence 2070.

After a hybridization step, the hybridized nucleic acid fragments are captured and may be amplified using PCR. For example, the target sequences 2070 can be enriched to obtain enriched sequences 2080 that can be subsequently sequenced. In some embodiments, each enriched sequence 2080 is replicated from a target sequence 2070. Enriched sequences 2080A and 2080C that are amplified from target sequences 2070A and 2070C, respectively, also include the thymine nucleotide base located near the edge of each sequence read 2080A or 2080C. As used hereafter, the mutated nucleotide base (e.g., thymine nucleotide base) in the enriched sequence 2080 that is mutated in relation to the reference allele (e.g., cytosine nucleotide base 2002) is considered as the alternative allele. Additionally, each enriched sequence 2080B amplified from target sequence 2070B includes the cytosine nucleotide base located near or at the center of each enriched sequence 2080B.

In block 1908, sequence reads are generated from the enriched DNA sequences, e.g., enriched sequences 2080 shown in FIG. 20. Sequencing data may be acquired from the enriched DNA sequences by known means in the art. For example, the method 1900 may include next generation sequencing (NGS) techniques including synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing is performed using sequencing-by-synthesis with reversible dye terminators.

In some embodiments, the sequence reads may be aligned to a reference genome using known methods in the art to determine alignment position information. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene.

In various embodiments, a sequence read is comprised of a read pair denoted as R₁ and R₂. For example, the first read R₁ may be sequenced from a first end of a nucleic acid fragment whereas the second read R₂ may be sequenced from the second end of the nucleic acid fragment. Therefore, nucleotide base pairs of the first read R₁ and second read R₂ may be aligned consistently (e.g., in opposite orientations) with nucleotide bases of the reference genome. Alignment position information derived from the read pair R₁ and R₂ may include a beginning position in the reference genome that corresponds to an end of a first read (e.g., R₁) and an end position in the reference genome that corresponds to an end of a second read (e.g., R₂). In other words, the beginning position and end position in the reference genome represent the likely location within the reference genome to which the nucleic acid fragment corresponds. An output file having SAM (sequence alignment map) format or BAM (binary) format may be generated and output for further analysis such as variant calling described above in conjunction with FIG. 2.

### CONCLUSION

Plural instances may be provided for components, operations, or structures described herein as a single instance. Finally, boundaries between various components, operations, and data stores are somewhat arbitrary, and particular operations are illustrated in the context of specific illustrative configurations. Other functional allocations are envisioned and may fall within the scope of the presently described implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

The terminology used in the present disclosure is intended to describe particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises,” “comprising,” “including,” “includes,” “having,” “has,” “with,” or variants thereof when used in this specification or claims, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event (” or “in response to detecting (the stated condition or event),” depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures, and techniques have not been shown in detail.

The foregoing description, for purpose of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, thereby enabling others skilled in the art to best utilize the implementations and various implementations with various modifications as are suited to the particular use contemplated.

What is claimed is:

1. A method of screening for a cancer condition in a test subject, the method comprising:
(a) obtaining a first biological sample from the test subject, wherein the first biological sample comprises cell-free nucleic acid from the test subject and potentially cell-free nucleic acid from at least one pathogen in a set of pathogens;
(b) sequencing the cell-free nucleic acid in the first biological sample to generate a plurality of sequence reads from the test subject;
(c) determining, for each respective pathogen in the set of pathogens, a corresponding amount of the plurality of sequence reads that map to a sequence in a pathogen target reference for the respective pathogen, thereby obtaining a set of amounts of sequence reads, each respective amount of sequence reads in the set of amounts of sequence reads for a corresponding pathogen in the set of pathogens; and
(d) using the set of amounts of sequence reads to determine whether the test subject has the cancer condition or a likelihood that the test subject has the cancer condition.

2. The method of claim 1, wherein the method further comprises:
evaluating the plurality of sequence reads to obtain an indication as to whether an APOBEC induced mutational signature associated with a first pathogen in the set of pathogens is present or absent; and wherein
the using (d) uses the indication as to whether the APOBEC induced mutational signature associated with the first pathogen is present or absent along with the set of amounts of sequence reads to determine whether the test subject has the cancer condition or the likelihood that the test subject has the cancer condition.

3. The method of claim 1, wherein the method further comprises:
evaluating, via k-mer analysis, the plurality of sequence reads to obtain an indication as to whether an APOBEC induced mutational signature is present or absent; and wherein
the using (d) uses the indication as to whether the APOBEC induced mutational signature associated with the first pathogen is present or absent along with the set of amounts of sequence reads to determine whether the test subject has the cancer condition or the likelihood that test subject has the cancer condition.

4. The method of any one of claims 2-3, wherein
the indication as to whether the APOBEC induced mutational signature associated with the first pathogen is present or absent further includes a measure of enrichment of the APOBEC induced mutational signature; and
the using (d) uses the measure of enrichment of the APOBEC induced mutational signature along with the set of amounts of sequence reads to determine whether the test subject has the cancer condition or the likelihood that the test subject has the cancer condition.

5. The method of any one of claims 2-3, wherein the APOBEC induced mutational signature is selected from either mutation signature type 2 or mutation signature type 13.

6. The method of any one of claims 1-5, wherein the method further comprises:
analyzing the first biological sample or a second biological sample from the test subject for an expression of an APOBEC protein associated with a first pathogen in the set of pathogens, and wherein
the using (d) uses the expression of the APOBEC protein and the set of amounts of sequence reads to determine whether the test subject has the cancer condition or the likelihood that the test subject has the cancer condition.

7. The method of claim 6, wherein the APOBEC protein is APOBEC1, APOBEC2, APOBEC3A, APOBEC3B, APOBEC3C, APOBEC3D, APOBEC3F, APOBEC3G, APOBEC3H, or APOBEC4.

8. The method of any one of claims 1-7, wherein the sequencing (b) is performed by whole genome sequencing, targeted panel sequencing, or whole genome bisulfite sequencing.

9. The method of any one of claims 1-8, wherein the pathogen target reference for the respective pathogen consists of a targeted panel of sequences from the reference genome for the respective pathogen and the determining (c) limits, for the respective pathogen, the mapping of each sequence read in the plurality of sequence reads to the corresponding targeted panel of sequences from the reference genome of the respective pathogen.

10. The method of claim 9, wherein the mapping comprises a sequence alignment between (i) one or more sequence reads in the plurality of sequence reads and (ii) a sequence in the pathogen target reference for the respective pathogen.

11. The method of any one of claims 1-8, wherein the pathogen target reference for the respective pathogen comprises a reference genome of the respective pathogen and the determining (c) aligns, for the respective pathogen, each sequence read in the plurality of sequence reads using the entire reference genome of the respective pathogen.

12. The method of any one of claims 1-11, wherein the set of pathogens is a single pathogen.

13. The method of any one of claims 1-11, wherein:
the set of pathogens is a plurality of pathogens, and
the determining (c) is performed for each respective pathogen in the plurality of pathogens.

14. The method of any one of claims 1-13, wherein the using (d) comprises:
determining a reference amount of sequence reads for a first pathogen in the set of pathogens associated with a predetermined percentile of a first distribution, wherein
each respective subject in a first cohort of subjects contributes to the first distribution an amount of sequence reads from the respective subject that map to a sequence in the pathogen target reference for the first pathogen, and
each subject in a first portion of the first cohort of subjects has the cancer condition, and
each subject in a second portion of the first cohort of subjects does not have the cancer condition, and
comparing (i) a first amount that is the amount of the plurality of sequence reads that map to a sequence in the pathogen target reference for the first pathogen from the test subject to (ii) a second amount that is the reference amount of sequence reads for the first pathogen in the set of pathogens associated with the predetermined percentile of the first distribution, wherein, when the first amount exceeds the second amount by a threshold amount the likelihood that the test subject has the cancer condition is adjusted or a determination is made that the test subject has the cancer condition.

15. The method of any one of claims 1-13, wherein the using (d) comprises:
determining a reference amount of sequence reads for a first pathogen in the set of pathogens associated with a predetermined percentile of a first distribution, wherein
each respective subject in a first cohort of subjects that do not have the cancer condition contributes to the first distribution an amount of sequence reads from the respective subject that map to a sequence in the pathogen target reference for the first pathogen,
thresholding the amount of the plurality of sequence reads that map to a sequence in the pathogen target reference for the first pathogen from the test subject by the reference amount of sequence reads for the first pathogen in the set of pathogens associated with the predetermined percentile of the first distribution to thereby form a scaled amount of the plurality of sequence reads, and
comparing (i) the scaled amount of the plurality of sequence reads to (ii) a scaled amount of the plurality of sequence reads associated with a predetermined percentile of a second distribution, wherein
each respective subject in a second cohort of subjects contributes to the second distribution a scaled amount of sequence reads from the respective subject that map to a sequence in the pathogen target reference for the first pathogen,
each subject in a first portion of the subjects in the second cohort have the cancer condition, and
each subject in a second portion of the subjects in the second cohort do not have the cancer condition.

16. The method of claim 15, wherein the test subject is deemed to have the cancer condition or the likelihood that the test subject has the cancer condition when the scaled amount of the plurality of sequence reads from the test subject exceeds the scaled amount of plurality of sequence reads associated with the predetermined percentile of the second distribution by a first predetermined cutoff value.

17. The method of any one of claims 1-13, wherein the using (d) comprises:
applying the set of amounts of sequence reads to a classifier to thereby determine either (i) whether the test subject has the cancer condition or (ii) the likelihood that test subject has the cancer condition.

18. The method of claim 17, the method further comprising:
training the classifier, prior to the using (d), by inputting into the classifier, for each respective subject in a first cohort of subjects, an amount of sequence reads from the respective subject that map to a sequence in the pathogen target reference for a respective pathogen in the set of pathogens,
wherein each subject in a first portion of the subjects in the first cohort have the cancer condition and each subject in a second portion of the subjects in the first cohort do not have the cancer condition.

19. The method of claim 17, the method further comprising:
training the classifier, prior to the using (d), by inputting into the classifier, for each respective subject in a first cohort of subjects, a normalized amount of sequence reads from the respective subject that map to a sequence in the pathogen target reference for a respective pathogen in the set of pathogens, wherein
each subject in a first portion of the subjects in the first cohort have the cancer condition,
each subject in a second portion of the subjects in the first cohort do not have the cancer condition,
the normalized amount of sequence reads from the respective subject that map to a sequence in the pathogen target reference for the respective pathogen is obtained by normalizing the amount of sequence reads from the respective subject that map to a sequence in the pathogen target reference for the respective pathogen by a reference amount of sequence reads for the respective pathogen associated with a predetermined percentile of a second distribution,
each respective subject in a second cohort of subjects that do not have the cancer condition contributes to the second distribution an amount of sequence reads from the respective subject that map to a sequence in the pathogen target reference for the respective pathogen.

20. The method of claim 18 or 19, wherein the classifier is a binomial classifier.

21. The method of claim 20, wherein the classifier is based on a logistic regression algorithm.

22. The method of claim 21, wherein the logistic regression algorithm provides a likelihood that the test subject has or does not have the cancer condition.

23. The method of claim 21, wherein the logistic regression algorithm provides a binomial assessment of whether the test subject has or does not have the cancer condition.

24. The method of claim 21, wherein
the logistic regression algorithm provides a plurality of likelihoods,
each respective likelihood in the plurality of likelihoods is a likelihood that the test subject has a corresponding cancer condition in a plurality of cancer conditions, and
the plurality of cancer conditions includes the cancer condition.

25. The method of claim 18 or 19, wherein the classifier is a multinomial classifier.

26. The method of claim 25, wherein the classifier is based on a logistic regression algorithm, a neural network algorithm, a support vector machine algorithm, or a decision tree algorithm.

27. The method of claim 1, the method further comprising:
performing an assay comprising measuring an amount of an APOBEC induced mutational signature of the cell-free nucleic acid in the first biological sample; and wherein
the using (d) comprises using the amount of the APOBEC induced mutational signature and the set of amounts of sequence reads to determine whether the test subject has the cancer condition or the likelihood that the test subject has the cancer condition.

28. The method of claim 1, the method further comprising:
obtaining a second biological sample from the test subject, wherein the second biological sample comprises cell-free nucleic acid from the test subject and potentially cell-free nucleic acid from a first pathogen in the set of pathogens; and
performing an assay comprising measuring an amount of an APOBEC induced mutational signature of the cell-free nucleic acid in the second biological sample; and wherein
the using (d) comprises using the amount of the APOBEC induced mutational signature and the set of amounts of sequence reads to determine whether the test subject has the cancer condition or the likelihood that the test subject has the cancer condition.

29. The method of any one of claims 27-28, wherein the APOBEC induced mutational signature is selected from either mutation signature type 2 or mutation signature type 13.

30. The method of any one of claims 1-29, wherein the test subject is human.

31. The method of any one of claims 1-30, wherein the cancer condition is cervical cancer, hepatocellular carcinoma, bladder cancer, breast cancer, esophageal cancer, prostate cancer, nasopharyngeal cancer, lung cancer, lymphoma, or leukemia.

32. The method of claim 31, wherein the cancer condition is early stage cancer.

33. The method of any one of claims 1-32, wherein the cancer condition is renal cancer, hepatocellular carcinoma, colorectal cancer, esophageal cancer, breast cancer, lung cancer, nasopharyngeal cancer, thyroid cancer, lymphoma, ovarian cancer, or cervical cancer.

34. The method of claim 33, wherein the cancer condition is late stage cancer.

35. The method of any one of claims 1-32, wherein the cancer condition is a liquid cancer, a liver cancer, or lung cancer.

36. The method of any one of claims 1-35, wherein the first biological sample and the second biological sample are plasma.

37. The method of any one of claims 1-35, wherein the first biological sample and the second biological sample are different aliquots of the same biological sample from the test subject.

38. The method of any one of claims 1-35, wherein the first biological sample and the second biological sample are the same biological sample.

39. The method of any one of claims 1-38, wherein the first biological sample comprises blood, whole blood, plasma, serum, urine, cerebrospinal fluid, fecal, saliva, sweat, tears, pleural fluid, pericardial fluid, or peritoneal fluid of the test subject.

40. The method of any one of claims 1-38, wherein the first biological sample consists of blood, whole blood, plasma, serum, urine, cerebrospinal fluid, fecal, saliva, sweat, tears, pleural fluid, pericardial fluid, or peritoneal fluid of the test subject.

41. The method of any one of claims 1-40, wherein a respective pathogen in the set of pathogens is Epstein-Barr virus (EBV), human cytomegalovirus (HCMV), hepatitis B virus (HBV), hepatitis C virus (HCV), human herpes virus (HHV), human mammary tumor virus (HMTV), human papillomavirus 16 (HPV16), human papillomavirus 18 (HPV18), human papillomavirus 60 (HPV-60), human papillomavirus ZM130 (HPV8-ZM130), human T-cell leukemia virus type 1 (HTLV-1), John Cunningham virus (JCV), molluscum contagiosum virus (MCV), or simian vacuolating virus 40 (SV40).

42. The method of any one of claims 1-40, wherein the set of pathogens is all or a subset of the RefSeq viral genome database.

43. The method of any one of claims 1-40, wherein the set of pathogens comprises any combination of the Epstein-Barr virus (EBV), human cytomegalovirus (HCMV), hepatitis B virus (HBV), hepatitis C virus (HCV), human herpes virus (HHV), human mammary tumor virus (HMTV), human papillomavirus 16 (HPV16), human papillomavirus 18 (HPV18), human papillomavirus 60 (HPV-60), human papillomavirus ZM130 (HPV8-ZM130), human T-cell leukemia virus type 1 (HTLV-1), John Cunningham virus (JCV), molluscum contagiosum virus (MCV), and simian vacuolating virus 40 (SV40).

44. The method of any one of claims 1-40, wherein the set of pathogens comprises any combination of human herpes virus 5 CINCY-TOWNE (HHV5-CINCY-TOWNE) virus, Epstein-Barr B95-8 (EBV-B95-8 virus), molluscum contagiosum virus R17b (MCV-R17b) virus, human papillomavirus 16 (HPV16) virus, human cytomegalovirus AD169 (HCMV-AD169) virus, hepatitis B virus (HBV) virus, hepatitis B virus 18 (HPV18) virus, hepatitis C virus (HCV) virus, human papillomavirus 8-ZM130 (HPV8-ZM130) virus, and John Cunningham virus PLYCG (JCV-PLYCG) virus.

45. The method of any one of claim 14-16 or 18-26, wherein the first cohort comprises twenty subjects.

46. The method of any one of claim 14-16 or 18-26, wherein the first cohort comprises one hundred subjects.

47. The method of any one of claims 14-16, wherein
the first cohort comprises twenty subjects, and
each respective subject in the first cohort contributes a percentage of sequence reads from the respective subject that map to a sequence in the pathogen target reference for the first pathogen to the first distribution.

48. The method of any one of claims 14-16, wherein
the first cohort comprises one hundred subjects, and
each respective subject in the first cohort contributes a percentage of sequence reads from the respective subject that map to a sequence in the pathogen target reference for the first pathogen to the first distribution.

49. The method of claim 18, wherein the amount of sequence reads from the respective subject that map to a sequence in the pathogen target reference for the respective pathogen is a percentage of the plurality of sequence reads measured from the respective subject that align to a sequence in the pathogen target reference of the respective pathogen.

50. The method of any one of claims 1-49, wherein the corresponding amount of the plurality of sequence reads that map to a sequence in the pathogen target reference for the respective pathogen is a percentage of the plurality of sequence reads from the test subject.

51. The method of claim 14, wherein the amount of sequence reads from the respective subject is a percentage of sequence reads measured from the respective subject that map to a sequence in the pathogen target reference for the first pathogen.

52. The method of any one of claims 14-16, wherein the predetermined percentile of the first distribution is the 95^{th} percentile.

53. The method of any one of claims 14-16, wherein the predetermined percentile of the first distribution is the 98^{th} percentile.

54. The method of claim 16, wherein the first predetermined cutoff value is zero.

55. The method of claim 16, wherein the first predetermined cutoff value is a single standard deviation away from a measure of central tendency of the second distribution.

56. The method of claim 16, wherein the first predetermined cutoff value is three standard deviations away from a measure of central tendency of the second distribution.

57. The method of claim 1, wherein
the set of pathogens comprises a first pathogen and a second pathogen,
the determining (c) comprises:
i) determining a first amount of the plurality of sequence reads that map to a sequence in a first pathogen target reference for the first pathogen,
ii) determining a second amount of the plurality of sequence reads that map to a sequence in a second pathogen target reference for the second pathogen,
iii) thresholding the first amount of the plurality of sequence reads from the test subject that map to a sequence in the first pathogen target reference by a first reference amount of sequence reads for the first pathogen associated with a first predetermined percentile of a first distribution to thereby form a scaled first amount of the plurality of sequence reads from the test subject, wherein each respective subject in a first cohort of subjects that do not have the cancer condition contributes to the first distribution an amount of sequence reads from the respective subject that map to a sequence in the first pathogen target reference for the first pathogen, and
iv) thresholding the second amount of the plurality of sequence reads from the test subject that map to a sequence in the second pathogen target reference by a second reference amount of sequence reads for the second pathogen associated with a second predetermined percentile of a second distribution to thereby determine a scaled second amount of the plurality of sequence reads from the test subject, wherein each respective subject in a second cohort of subjects that do not have the cancer condition contributes to the second distribution an amount of sequence reads from the respective subject that map to a sequence in the second pathogen target reference for the second pathogen, and wherein
the using (d) deems the test subject to have the cancer condition or a likelihood that the test subject has the cancer condition when a classifier inputted with at least the scaled first amount and the scaled second amount indicates that the test subject has the cancer condition.

58. The method of claim 57, wherein,
the classifier is based on a logistic regression algorithm,
the logistic regression individually weights the scaled first amount based on an amount of sequence reads mapping to a sequence in the first pathogen target reference observed in a training cohort of subjects that includes subjects that have the cancer condition and subjects that do not have the cancer condition, and
the logistic regression individually weights the scaled second amount based on an amount of sequence reads mapping to a sequence in the second pathogen target reference observed in the training cohort.

59. The method of claim 1, wherein:
the determining (c) comprises thresholding the corresponding amount of the plurality of sequence reads that map to a sequence in the pathogen target reference for the respective pathogen based on an amount of sequence reads associated with a predetermined percentile of a respective distribution, wherein each respective subject in a respective cohort of subjects that do not have the cancer condition contributes to the respective distribution an amount of sequence reads from the respective subject that map to a sequence in the pathogen target reference for the respective pathogen, thereby determining a scaled respective amount of the plurality of sequence reads from the test subject, and wherein
the using (c) deems the test subject to have the cancer condition or the likelihood that the test subject has the cancer condition when a classifier inputted with at least each scaled respective amount of the plurality of sequence reads from the test subject indicates that the test subject has the cancer condition.

60. The method of claim 59, wherein:
the classifier is based on a logistic regression algorithm that individually weights each scaled respective amount of the plurality of sequence reads based on a corresponding amount of sequence reads mapping to a sequence in the pathogen target reference of the corresponding pathogen observed in a training cohort of subjects that includes subjects that have the cancer condition and subjects that do not have the cancer condition.

61. The method of claim 59, wherein the set of pathogens comprises between two and one hundred pathogens.

62. The method of claim 57 or 59, wherein the classifier is based on a logistic regression algorithm, a neural network algorithm, a support vector machine algorithm, or a decision tree algorithm that has been trained on a training cohort of subjects that includes subjects that have the cancer condition and subjects that do not have the cancer condition.

63. The method of claim 1, wherein
the determining (c) comprises thresholding the corresponding amount of the plurality of sequence reads from the test subject that map to a sequence in the pathogen target reference for the respective pathogen on an amount of sequence reads associated with a predetermined percentile of a respective distribution, wherein each respective subject in a respective cohort of subjects that do not have the cancer condition contributes to the respective distribution an amount of sequence reads from the respective subject that map to a sequence in the pathogen target reference for the respective pathogen, thereby determining a scaled respective amount of the plurality of sequence reads from the test subject, and
the using (d) sums each scaled respective amount of the plurality of sequence reads from the test subject to determine an overall oncopathogen load, wherein the using (d) indicates that the test subject has the cancer condition or the likelihood that the test subject has the cancer condition when the overall oncopathogen load satisfies a threshold cutoff condition.

64. The method of claim 1, wherein
the using (d) calls the test subject as having the cancer condition or the likelihood that the test subject has the cancer condition when the set of amounts of sequence reads exceeds a threshold cutoff condition that is a predetermined specificity for overall oncopathogen load across the set of pathogens determined for a pool of subjects that do not have the cancer condition.

65. The method of claim 64, wherein the predetermined specificity is the 95^{th} percentile.

66. The method of any one of claims 1-65, wherein
the determining a corresponding amount of the plurality of sequence reads that map to a sequence in the pathogen target reference for the respective pathogen comprises translating the plurality of sequence reads from the test subject in a reading frame to form a plurality of translated sequence reads and comparing the plurality of translated sequence reads to a translation of each sequence in the pathogen target reference.

67. The method of any one of claims 1-66, wherein
the determining a corresponding amount of the plurality of sequence reads that map to a sequence in the pathogen target reference for the respective pathogen comprises k-mer matching the plurality of sequence reads from the test subject to the pathogen target reference in nucleic acid, ribonucleic acid, or protein space.

68. The method of any one of claims 1-67, wherein
the test subject is human, and
the method further comprises performing an end-point analysis of the corresponding amount of the plurality of sequence reads within the human genome, and
the using (d) further uses the end-point analysis to determine whether the test subject has the cancer condition or a likelihood that the test subject has the cancer condition.

69. The method of any one of claims 1-68, further comprising:
(e) providing a therapeutic intervention or imaging of the test subject based on the determination of whether the test subject has the cancer condition or the likelihood that the test subject has the cancer condition of step (d).

70. A method of screening for a cancer condition in a test subject, the method comprising:
(a) obtaining a first biological sample from the test subject, wherein the first biological sample comprises cell-free nucleic acid from the test subject and potentially cell-free nucleic acid from at least one pathogen in a set of pathogens;
(b) performing a first assay comprising measuring an amount of a first feature of the cell-free nucleic acid in the first biological sample;
(c) performing a second assay comprising:
i. sequencing the cell-free nucleic acid in a second biological sample to generate a plurality of sequence reads from the test subject, wherein the second biological sample is from the test subject, and wherein the second biological sample comprises cell-free nucleic acid from the test subject and potentially cell-free nucleic acid from at least one pathogen in the set of pathogens, and
ii. determining, for each respective pathogen in the set of pathogens, a corresponding amount of the plurality of sequence reads that map to a sequence in a pathogen target reference for the respective pathogen, thereby obtaining a set of amounts of sequence reads, each respective amount of sequence reads in the set of amounts of sequence reads for a corresponding pathogen in the set of pathogens; and
(d) screening for the cancer condition based on step (b) and step (c), wherein the test subject is deemed to have a likelihood of having the cancer condition or to have the cancer condition when either the first assay or the second assay, or both the first assay and the second assay, indicate that the test subject has or does not have the cancer condition or provides a likelihood that the test subject has or does not have the cancer condition.

71. The method of claim 70, wherein the method further comprises:
evaluating the plurality of sequence reads to obtain an indication as to whether an APOBEC induced mutational signature associated with a first pathogen in the set of pathogens is present or absent; and wherein
the screening (d) uses (i) the indication as to whether the signature fragment signature associated with a first pathogen is present or absent, (ii) the amount of the first feature, and (iii) the indication as to whether the APOBEC induced mutational signature associated with the first pathogen is present or absent to determine whether the test subject has the cancer condition or the likelihood that the test subject has the cancer condition.

72. The method of claim 70, wherein the method further comprises:
evaluating, via k-mer analysis, the plurality of sequence reads to obtain an indication as to whether an APOBEC induced mutational signature is present or absent; and wherein
the screening (d) uses (i) the indication as to whether the signature fragment signature associated with a first pathogen is present or absent, (ii) the amount of the first feature, and (iii) the indication as to whether the APOBEC induced mutational signature associated with the first pathogen is present or absent to determine whether the test subject has the cancer condition or the likelihood that the test subject has the cancer condition.

73. The method of any one of claims 71-73, wherein
the indication as to whether the APOBEC induced mutational signature associated with the first pathogen is present or absent further includes a measure of enrichment of the APOBEC induced mutational signature; and
the screening (d) uses (i) the indication as to whether the signature fragment signature associated with a first pathogen is present or absent, (ii) the amount of the first feature, and (iii) the measure of enrichment of the APOBEC induced mutational signature to determine whether the test subject has the cancer condition or the likelihood that the test subject has the cancer condition.

74. The method of any one of claims 70-73, wherein the method further comprises:
analyzing the first biological sample or a second biological sample from the test subject for an expression of an APOBEC protein associated with a first pathogen in the set of pathogens, and wherein
the screening (d) uses (i) the indication as to whether the signature fragment signature associated with a first pathogen is present or absent, (ii) the amount of the first feature, and (iii) the expression of the APOBEC protein associated with the first pathogen to determine whether the test subject has the cancer condition or the likelihood that the test subject has the cancer condition.

75. The method of claim 74, wherein the APOBEC protein is APOBEC1, APOBEC2, APOBEC3A, APOBEC3B, APOBEC3C, APOBEC3D, APOBEC3F, APOBEC3G, APOBEC3H, or APOBEC4.

76. The method of any one of claims 70-75, the method further comprising:
performing a third assay comprising measuring an amount of an APOBEC induced mutational signature of the cell-free nucleic acid in the first biological sample; and wherein
the screening (d) uses (i) the indication as to whether the signature fragment signature associated with a first pathogen is present or absent, (ii) the amount of the first feature, and (iii) the amount of the APOBEC induced mutational signature to determine whether the test subject has the cancer condition or the likelihood that the test subject has the cancer condition.

77. The method of any one of claims 70-75, wherein performing the second assay further comprises:
measuring an amount of an APOBEC induced mutational signature of the cell-free nucleic acid in the second biological sample; and wherein
the screening (d) uses (i) the indication as to whether the signature fragment signature associated with a first pathogen is present or absent, (ii) the amount of the first feature, and (iii) the amount of the APOBEC induced mutational signature to determine whether the test subject has the cancer condition or the likelihood that the test subject has the cancer condition.

78. The method of any one of claims 71-77, wherein the APOBEC induced mutational signature is selected from either mutation signature type 2 or mutation signature type 13.

79. The method of claim 70, wherein the sequencing (c)(i) is performed by whole genome sequencing, targeted panel sequencing, or whole genome bisulfite sequencing.

80. The method of claim 70, wherein the test subject is human.

81. The method of any one of claims 70-80, wherein the cancer condition is cervical cancer, hepatocellular carcinoma, bladder cancer, breast cancer, esophageal cancer, prostate cancer, nasopharyngeal cancer, lung cancer, lymphoma, or leukemia.

82. The method of claim 81, wherein the cancer condition is early stage cancer.

83. The method of claim 70 or 80, wherein the cancer condition is renal cancer, hepatocellular carcinoma, colorectal cancer, esophageal cancer, breast cancer, lung cancer, nasopharyngeal cancer, thyroid cancer, lymphoma, ovarian cancer, or cervical cancer.

84. The method of claim 83, wherein the cancer condition is late stage cancer.

85. The method of claim 70, wherein the cancer condition is a liquid cancer, a liver cancer, or lung cancer.

86. The method of any one of claims 70-85, wherein the first biological sample and the second biological sample are plasma.

87. The method of any one of claims 70-85, wherein the first biological sample and the second biological sample are different aliquots of the same biological sample from the test subject.

88. The method of any one of claims 70-85, wherein the first biological sample and the second biological sample are the same biological sample.

89. The method of any one of claims 70-88, wherein the first biological sample or the second biological sample comprises blood, whole blood, plasma, serum, urine, cerebrospinal fluid, fecal, saliva, sweat, tears, pleural fluid, pericardial fluid, or peritoneal fluid of the test subject.

90. The method of any one of claims 70-88, wherein the first biological sample or the second biological sample consists of blood, whole blood, plasma, serum, urine, cerebrospinal fluid, fecal, saliva, sweat, tears, pleural fluid, pericardial fluid, or peritoneal fluid of the test subject.

91. The method of any one of claims 70-90, wherein the respective pathogen is Epstein-Barr virus (EBV), human cytomegalovirus (HCMV), hepatitis B virus (HBV), hepatitis C virus (HCV), human herpes virus (HHV), human mammary tumor virus (HMTV), human papillomavirus 16 (HPV16), human papillomavirus 18 (HPV18), human papillomavirus 60 (HPV-60), human papillomavirus ZM130 (HPV8-ZM130), human T-cell leukemia virus type 1 (HTLV-1), John Cunningham virus (JCV), molluscum contagiosum virus (MCV), or simian vacuolating virus 40 (SV40).

92. The method of any one of claims 70-90, wherein the set of pathogens is all or a subset of the RefSeq viral genome database.

93. The method of any one of claims 70-90, wherein the set of pathogens comprises any combination of the Epstein-Barr virus (EBV), human cytomegalovirus (HCMV), hepatitis B virus (HBV), hepatitis C virus (HCV), human herpes virus (HHV), human mammary tumor virus (HMTV), human papillomavirus 16 (HPV16), human papillomavirus 18 (HPV18), human papillomavirus 60 (HPV-60), human papillomavirus ZM130 (HPV8-ZM130), human T-cell leukemia virus type 1 (HTLV-1), John Cunningham virus (JCV), molluscum contagiosum virus (MCV), and simian vacuolating virus 40 (SV40).

94. The method of any one of claims 70-93, wherein
the test subject is human, and
the first feature is somatic copy number alteration count across a targeted panel of genes in the human genome.

95. The method of claim 94, wherein the targeted panel of genes consists of between twenty and six hundred genes.

96. The method of any one of claims 70-93, wherein
the test subject is human, and
the first feature is somatic copy number alteration count across the human genome.

97. The method of any one of claims 70-93, wherein
the test subject is human, and
the first feature is a single nucleotide variant count, an insertion mutation count, a deletion mutation count, or a nucleic acid rearrangement count across a targeted panel of genes in the human genome.

98. The method of any one of claims 70-90, wherein the set of pathogens comprises any combination of human herpes virus 5 CINCY-TOWNE (HHV5-CINCY-TOWNE) virus, Epstein-Barr B95-8 (EBV-B95-8 virus), molluscum contagiosum virus R17b (MCV-R17b) virus, human papillomavirus 16 (HPV16) virus, human cytomegalovirus AD169 (HCMV-AD169) virus, hepatitis B virus (HBV) virus, hepatitis B virus 18 (HPV18) virus, hepatitis C virus (HCV) virus, human papillomavirus 8-ZM130 (HPV8-ZM130) virus, and John Cunningham virus PLYCG (JCV-PLYCG) virus.

99. The method of claim 70, wherein the pathogen target reference for the respective pathogen consists of a corresponding targeted panel of sequences from the reference genome for the respective pathogen and the performing (c)(ii) limits, for the respective pathogen, the mapping of each sequence read in the plurality of sequence reads to the corresponding targeted panel of sequences from the reference genome of the respective pathogen.

100. The method of claim 99, wherein the mapping comprises a sequence alignment between (i) one or more sequence reads in the plurality of sequence reads and (ii) a sequence in the corresponding targeted panel of sequences from the reference genome of the respective pathogen.

101. The method of claim 99, wherein the mapping comprises a comparison of a methylation pattern between (i) one or more sequence reads in the plurality of sequence reads and (ii) a sequence in the corresponding targeted panel of sequences from the reference genome of the respective pathogen.

102. The method of claim 70, wherein the pathogen target reference comprises a reference genome of the respective pathogen or a portion thereof, and the performing (c)(ii) aligns, for the respective pathogen, one or more sequence reads in the plurality of sequence reads using the entire reference genome of the respective pathogen.

103. The method of claim 70, wherein the pathogen target reference is a reference genome of the respective pathogen or a portion thereof, and the performing (c)(ii) compares, for the respective pathogen, a methylation pattern of one or more sequence reads in the plurality of sequence reads to a methylation pattern across the entire reference genome of the respective pathogen.

104. The method of any one of claims 70-103, wherein the set of pathogens is a single pathogen.

105. The method of any one of claims 70-103, wherein
the set of pathogens comprises a plurality of pathogens, and
the performing (c)(ii) is performed for each respective pathogen in the plurality of pathogens.

106. The method of any one of claims 70-105, wherein the second assay further comprises:
determining a reference amount of sequence reads for a first pathogen in the set of pathogens associated with a predetermined percentile of a first distribution, wherein
each respective subject in a first cohort of subjects contributes to the first distribution an amount of sequence reads from the respective subject that map to a sequence in the pathogen target reference for the first pathogen, wherein each subject in a first portion of the first cohort of subjects has the cancer condition and each subject in a second portion of the first cohort of subjects does not have the cancer condition, and
comparing (i) a first amount that is the amount of the plurality of sequence reads that map to a sequence in a pathogen target reference for the first pathogen from the test subject to (ii) a second amount that is the reference amount of sequence reads for the first pathogen in the set of pathogens associated with the predetermined percentile of the first distribution, wherein, when the first amount exceeds the second amount by a threshold amount the second assay dictates a likelihood that the test subject has the cancer condition or determines that the test subject has the cancer condition.

107. The method of any one of claims 70-105, wherein the second assay further comprises:
determining a reference amount of sequence reads for a first pathogen in the set of pathogens associated with a predetermined percentile of a first distribution, wherein
each respective subject in a first cohort of subjects that do not have the cancer condition contributes to the first distribution an amount of sequence reads from the respective subject that map to a sequence in the pathogen target reference for the first pathogen,
thresholding the amount of the plurality of sequence reads that map to a sequence in a pathogen target reference for the first pathogen from the test subject by the reference amount of sequence reads for the first pathogen in the set of pathogens associated with the predetermined percentile of the first distribution to thereby form a scaled amount of the plurality of sequence reads, and
comparing (i) the scaled amount of the plurality of sequence reads to (ii) a scaled amount of the plurality of sequence reads associated with a predetermined percentile of a second distribution, wherein each respective subject in a second cohort of subjects contributes to the second distribution a scaled amount of sequence reads from the respective subject that map to a sequence in the pathogen target reference for the first pathogen, wherein each subject in a first portion of the subjects in the second cohort have the cancer condition and each subject in a second portion of the subjects in the second cohort do not have the cancer condition.

108. The method of claim 107, wherein the first cohort comprises twenty subjects that each contribute an amount of sequence reads from the respective subject that map to a sequence in the pathogen target reference for the first pathogen to the first distribution.

109. The method of claim 107, wherein the first cohort comprises one hundred subjects that each contribute an amount of sequence reads from the respective subject that map to a sequence in the pathogen target reference for the first pathogen to the first distribution.

110. The method of claim 107, wherein the predetermined percentile for the first distribution is the 95^{th} percentile.

111. The method of claim 107, wherein the predetermined percentile for the first distribution is the 98^{th} percentile.

112. The method of claim 70, wherein
the determining (c)(ii) determines a corresponding first amount of the plurality of sequence reads that map to a sequence in a pathogen target reference for a first pathogen,
the determining (c)(ii) determines a corresponding second amount of the plurality of sequence reads that map to a sequence in a pathogen target reference for a second pathogen,
the first amount is thresholded on an amount of sequence reads associated with a predetermined percentile of a first distribution, wherein each respective subject in a first cohort of subjects that do not have the cancer condition contributes to the first distribution an amount of sequence reads from the respective subject that map to a sequence in the pathogen target reference for the first pathogen, thereby determining a scaled first amount of the plurality of sequence reads from the test subject,
the second amount is thresholded on an amount of sequence reads associated with a predetermined percentile of a second distribution, wherein each respective subject in a second cohort of subjects that do not have the cancer condition contributes to the second distribution an amount of sequence reads from the respective subject that map to a sequence in the pathogen target reference for the second pathogen, thereby determining a scaled second amount of the plurality of sequence reads from the test subject, and
the second assay indicates that the test subject has or does not have the cancer condition or provides a likelihood that the test subject has or does not have the cancer condition based, at least in part, on the scaled first amount and the scaled second amount.

113. The method of claim 112, wherein the test subject is deemed by the second assay to have or not have the cancer condition or the second assay provides a likelihood that the test subject has or does not have the cancer by inputting at least the scaled first amount of the plurality of sequence reads and the scaled second amount of the plurality of sequence reads into a classifier.

114. The method of claim 113, wherein,
the classifier is a logistic regression,
the logistic regression individually weights the scaled first amount of the plurality of sequence reads based on an amount of sequence reads mapping to a sequence in the pathogen target reference for the first pathogen observed in a training cohort of subjects that includes subjects that have the cancer condition and subjects that do not have the cancer condition, and
the logistic regression individually weights the scaled second amount of the plurality of sequence reads based on an amount of sequence reads mapping to a sequence in the pathogen target reference for the second pathogen observed in the training cohort.

115. The method of any one of claims 70-105, wherein the performing (c) further comprises:
applying the corresponding amount of sequence reads that map to a sequence in the pathogen target reference for the respective pathogen to a classifier to thereby have the second assay call either (i) whether the test subject has the cancer condition or (ii) a likelihood that test subject has the cancer condition.

116. The method of claim 115, wherein the applying also applies the amount of the first feature to the classifier.

117. The method of claim 115, the method further comprising:
training the classifier, prior to the performing (c), by inputting into the classifier, for each respective subject in a first cohort of subjects, an amount of sequence reads from the respective subject that map to a sequence in the pathogen target reference for the respective pathogen,
wherein each subject in a first portion of the subjects in the first cohort have the cancer condition and each subject in a second portion of the subjects in the first cohort do not have the cancer condition.

118. The method of claim 115, the method further comprising:
training the classifier, prior to the performing (c), by inputting into the classifier, for each respective subject in a first cohort of subjects, a normalized amount of sequence reads from the respective subject that map to a sequence in the pathogen target reference for the respective pathogen, wherein
each subject in a first portion of the subjects in the first cohort have the cancer condition,
each subject in a second portion of the subjects in the first cohort do not have the cancer condition,
the normalized amount of sequence reads from the respective subject that map to a sequence in the pathogen target reference for the respective pathogen is obtained by normalizing the amount of sequence reads from the respective subject that map to a sequence in the pathogen target reference for the respective pathogen by a reference amount of sequence reads for the respective pathogen associated with a predetermined percentile of a second distribution,
each respective subject in a second cohort of subjects that do not have the cancer condition contributes to the second distribution an amount of sequence reads from the respective subject that map to a sequence in the pathogen target reference for the respective pathogen.

119. The method of claim 117 or 118, wherein the classifier is a binomial classifier.

120. The method of claim 119, wherein the classifier is a logistic regression.

121. The method of claim 120, wherein the logistic regression algorithm provides a likelihood that the test subject has or does not have the cancer condition.

122. The method of claim 120, wherein the logistic regression algorithm provides a binomial assessment of whether the test subject has or does not have the cancer condition.

123. The method of claim 120, wherein
the logistic regression algorithm provides a plurality of likelihoods,
each respective likelihood in the plurality of likelihoods is a likelihood that the test subject has a corresponding cancer condition in a plurality of cancer conditions, and
the plurality of cancer conditions includes the cancer condition.

124. The method of claim 117 or 118, wherein the classifier is a multinomial classifier.

125. The method of claim 124, wherein the classifier is based on a logistic regression algorithm, a neural network algorithm, a support vector machine algorithm, or a decision tree algorithm.

126. The method of any one of claims 70-125, wherein the method further comprises:
evaluating the plurality of sequence reads to obtain an indication as to whether a sequence fragment signature associated with a first pathogen in the set of pathogens is present or absent; and wherein
the screening (d) uses (i) the indication as to whether the signature fragment signature associated with a first pathogen is present or absent, (ii) the amount of the first feature, and (iii) the set of amounts of sequence reads to determine whether the test subject has the cancer condition or the likelihood that test subject has the cancer condition.

127. The method of any one of claims 70-125, wherein the method further comprises:
evaluating the plurality of sequence reads to obtain an indication as to whether a methylation signature associated with a first pathogen in the set of pathogens is present or absent; and wherein
the screening (d) uses the (i) indication as to whether the methylation signature associated with a first pathogen is present or absent, (ii) the amount of the first feature, and (iii) the set of amounts of sequence reads to determine whether the test subject has the cancer condition or the likelihood that test subject has the cancer condition.

128. The method of any one of claims 70-125, wherein the method further comprises:
evaluating the plurality of sequence reads to obtain an indication as to whether a sequence fragment signature associated with a first pathogen in the set of pathogens is present or absent; and
evaluating the plurality of sequence reads to obtain an indication as to whether a methylation signature associated with the first pathogen in the set of pathogens is present or absent; and wherein
the screening (d) uses (i) the indication as to whether the signature fragment signature associated with the first pathogen is present or absent, (ii) an indication as to whether a methylation signature associated with the first pathogen is present or absent, (iii) the amount of the first feature, and (iv) the set of amounts of sequence reads to determine whether the test subject has the cancer condition or the likelihood that test subject has the cancer condition.

129. The method of claim 70, wherein
the performing (c) further comprises, for each respective pathogen in the set of pathogens, thresholding the corresponding amount of the plurality of sequence reads that map to a sequence in the pathogen target reference for the respective pathogen on an amount of sequence reads associated with a predetermined percentile of a respective distribution, wherein each respective subject in a respective cohort of subjects that do not have the cancer condition contributes to the respective distribution an amount of sequence reads from the respective subject that map to a sequence in the pathogen target reference for the respective pathogen, thereby determining a scaled respective amount of the plurality of sequence reads from the test subject, and
the test subject is deemed by the second assay to have the likelihood of having the cancer condition or to have the cancer condition when a classifier inputted with at least each scaled respective amount of the plurality of sequence reads from the test subject indicates that the test subject has the cancer condition.

130. The method of claim 129, wherein,
the classifier is a logistic regression that individually weights each scaled respective amount of the plurality of sequence reads based on a corresponding amount of sequence reads mapping a sequence in the pathogen target reference for the respective pathogen observed in a training cohort of subjects that includes subjects that have the cancer condition and subjects that do not have the cancer condition.

131. The method of claim 129, wherein the set of pathogens comprises between two and one hundred pathogens.

132. The method of claim 129, wherein the classifier is based on a logistic regression algorithm, a neural network algorithm, a support vector machine algorithm, or a decision tree algorithm that has been trained on a training cohort of subjects that includes subjects that have the cancer condition and subjects that do not have the cancer condition.

133. The method of claim 70, wherein
the performing (c) further comprises, for each respective pathogen in the set of pathogens, thresholding the corresponding amount of the plurality of sequence reads that map to a sequence in the pathogen target reference for the respective pathogen on an amount of sequence reads associated with a predetermined percentile of a respective distribution, wherein each respective subject in a respective cohort of subjects that do not have the cancer condition contributes to the respective distribution an amount of sequence reads from the respective subject that map to a sequence in the pathogen target reference for the respective pathogen, thereby determining a scaled respective amount of the plurality of sequence reads from the test subject,
summing each scaled respective amount of the plurality of sequence reads from the test subject to determine an overall oncopathogen load, and wherein the second assay indicates that the test subject has the cancer condition when the overall oncopathogen load satisfies a threshold cutoff condition.

134. The method of claim 133, wherein the threshold cutoff condition is a predetermined specificity for overall oncopathogen load across the set of pathogens determined for a pool of subjects that do not have the cancer condition.

135. The method of claim 134, wherein the predetermined specificity is the 95^{th} percentile.

136. The method of claim 70, wherein
the first assay has a sensitivity for a first set of markers indicative of the cancer condition, and
the first feature is one of a copy number, a fragment size distribution, a fragmentation pattern, a methylation status, or a mutational status of the cell-free nucleic acid in the first biological sample across the first set of markers.

137. The method of claim 136, wherein
the amount of the first feature is thresholded on an amount of the first feature associated with a predetermined percentile of a second distribution to thereby form a scaled amount of the first feature, wherein each respective subject in a second cohort of subjects that do not have the cancer condition contributes to the second distribution a value for the first feature measured from the respective subject, and
the test subject is deemed by the first assay to have the cancer condition when the scaled amount of the first feature exceeds the amount of the first feature associated with the predetermined percentile of the second distribution by a second predetermined cutoff value.

138. The method of claim 137, wherein the second predetermined cutoff value is zero.

139. The method of claim 137, wherein the second predetermined cutoff value is a single standard deviation greater than a measure of central tendency of the second distribution.

140. The method of claim 137, wherein the second predetermined cutoff value is three standard deviations greater than a measure of central tendency of the second distribution.

141. The method of claim 70, wherein the corresponding amount of the plurality of sequence reads that map to a sequence in a pathogen target reference for the respective pathogen is a percentage of the plurality of sequence reads from the test subject that map to a sequence in a pathogen target reference for the respective pathogen measured in the second biological sample.

142. The method of any one of claims 70-141, wherein the determining a corresponding amount of the plurality of sequence reads that map to a sequence in a pathogen target reference for the corresponding pathogen comprises translating the plurality of sequence reads in a reading frame to form a plurality of translated sequence reads and comparing the plurality of translated sequence reads to a translation of the pathogen target reference.

143. The method of any one of claims 70-141, wherein the determining a corresponding amount of the plurality of sequence reads that map to a sequence in a pathogen target reference for the corresponding pathogen comprises k-mer matching the plurality of sequence reads to the pathogen target reference in nucleic acid, ribonucleic acid or protein space.

144. The method of any one of claims 70-143, wherein
the test subject is human, and
the second assay further comprises performing an end-point analysis of each respective amount of the plurality of sequence reads within the human genome.

145. The method of any one of claims 70-144, further comprising providing a therapeutic intervention or imaging of the test subject based on an outcome of the screening step (d).

146. A method of screening for a cancer condition in a test subject, the method comprising:
(a) obtaining a first biological sample from the test subject, wherein the first biological sample comprises cell-free nucleic acid from the test subject and potentially cell-free nucleic acid from at least one pathogen in a set of pathogens;
(b) sequencing the cell-free nucleic acid in the biological sample to generate a plurality of sequence reads from the test subject;
(c) evaluating the plurality of sequence reads to obtain an indication as to whether a sequence fragment signature associated with a respective pathogen in the set of pathogens is present or absent; and
(d) using the indication as to whether the signature fragment signature associated with the respective pathogen is present or absent to determine whether the test subject has the cancer condition or the likelihood that test subject has the cancer condition.

147. The method of claim 146, wherein the method further comprises:
evaluating the plurality of sequence reads to obtain an indication as to whether an APOBEC induced mutational signature associated with a first pathogen in the set of pathogens is present or absent; and wherein
the using (d) uses the indication as to whether the APOBEC induced mutational signature associated with the first pathogen is present or absent along with the indication as to whether the signature fragment signature associated with the respective pathogen is present or absent to determine whether the test subject has the cancer condition or the likelihood that the test subject has the cancer condition.

148. The method of claim 146, wherein the method further comprises:
evaluating, via k-mer analysis, the plurality of sequence reads to obtain an indication as to whether an APOBEC induced mutational signature is present or absent; and wherein
the using (d) uses the indication as to whether the APOBEC induced mutational signature associated with the first pathogen is present or absent along with the indication as to whether the signature fragment signature associated with the respective pathogen is present or absent to determine whether the test subject has the cancer condition or the likelihood that the test subject has the cancer condition.

149. The method of any one of claims 147-148, wherein
the indication as to whether the APOBEC induced mutational signature associated with the first pathogen is present or absent further includes a measure of enrichment of the APOBEC induced mutational signature; and
the using (d) uses the measure of enrichment of the APOBEC induced mutational signature along with the indication as to whether the signature fragment signature associated with the respective pathogen is present or absent to determine whether the test subject has the cancer condition or the likelihood that the test subject has the cancer condition.

150. The method of any one of claims 146-149, wherein the method further comprises:
analyzing the first biological sample or a second biological sample from the test subject for an expression of an APOBEC protein associated with a first pathogen in the set of pathogens, and wherein
the using (d) uses the expression of the APOBEC protein along with the indication as to whether the signature fragment signature associated with the respective pathogen is present or absent to determine whether the test subject has the cancer condition or the likelihood that the test subject has the cancer condition.

151. The method of claim 150, wherein the APOBEC protein is APOBEC1, APOBEC2, APOBEC3A, APOBEC3B, APOBEC3C, APOBEC3D, APOBEC3F, APOBEC3G, APOBEC3H, or APOBEC4.

152. The method of any one of claims 146-151, the method further comprising:
performing an assay comprising measuring an amount of an APOBEC induced mutational signature of the cell-free nucleic acid in the first biological sample; and wherein
the using (d) comprises using the amount of the APOBEC induced mutational signature and the set of amounts of sequence reads to determine whether the test subject has the cancer condition or the likelihood that the test subject has the cancer condition.

153. The method of claim 146, the method further comprising:
obtaining a second biological sample from the test subject, wherein the second biological sample comprises cell-free nucleic acid from the test subject and potentially cell-free nucleic acid from a first pathogen in the set of pathogens; and
performing an assay comprising measuring an amount of an APOBEC induced mutational signature of the cell-free nucleic acid in the second biological sample; and wherein
the using (d) comprises using the amount of the APOBEC induced mutational signature and the set of amounts of sequence reads to determine whether the test subject has the cancer condition or the likelihood that the test subject has the cancer condition.

154. The method of any one of claims 147-153, wherein the APOBEC induced mutational signature is selected from either mutation signature type 2 or mutation signature type 13.

155. The method of claim 146, wherein the sequencing (b) is performed by whole genome sequencing, targeted panel sequencing, or whole genome bisulfite sequencing.

156. The method of claim 146, wherein the test subject is human.

157. The method of claim 156, wherein the cancer condition is cervical cancer, hepatocellular carcinoma, bladder cancer, breast cancer, esophageal cancer, prostate cancer, nasopharyngeal cancer, lung cancer, lymphoma, or leukemia.

158. The method of claim 157, wherein the cancer condition is early stage cancer.

159. The method of claim 156, wherein the cancer condition is renal cancer, hepatocellular carcinoma, colorectal cancer, esophageal cancer, breast cancer, lung cancer, nasopharyngeal cancer, thyroid cancer, lymphoma, ovarian cancer, or cervical cancer.

160. The method of claim 159, wherein the cancer condition is late stage cancer.

161. The method of claim 146, wherein the cancer condition is a liquid cancer, a liver cancer, or lung cancer.

162. The method of any one of claims 146-161, wherein the first biological sample and the second biological sample are plasma.

163. The method of any one of claims 146-161, wherein the sample and the second biological sample are different aliquots of the same biological sample from the test subject.

164. The method of any one of claims 146-161, wherein the first biological sample and the second biological sample are the same biological sample.

165. The method of any one of claims 146-161, wherein the first biological sample or the second biological sample comprises blood, whole blood, plasma, serum, urine, cerebrospinal fluid, fecal, saliva, sweat, tears, pleural fluid, pericardial fluid, or peritoneal fluid of the test subject.

166. The method of any one of claims 146-161, wherein the first biological sample or the second biological sample consists of blood, whole blood, plasma, serum, urine, cerebrospinal fluid, fecal, saliva, sweat, tears, pleural fluid, pericardial fluid, or peritoneal fluid of the test subject.

167. The method of any one of claims 146-166, wherein a respective pathogen in the set of pathogens is Epstein-Barr virus (EBV), human cytomegalovirus (HCMV), hepatitis B virus (HBV), hepatitis C virus (HCV), human herpes virus (HHV), human mammary tumor virus (HMTV), human papillomavirus 16 (HPV16), human papillomavirus 18 (HPV18), human papillomavirus 60 (HPV-60), human papillomavirus ZM130 (HPV8-ZM130), human T-cell leukemia virus type 1 (HTLV-1), John Cunningham virus (JCV), molluscum contagiosum virus (MCV), or simian vacuolating virus 40 (SV40).

168. The method of any one of claims 146-166, wherein the set of pathogens is all or a subset of the RefSeq viral genome database.

169. The method of any one of claims 146-166, wherein the set of pathogens comprises any combination of the Epstein-Barr virus (EBV), human cytomegalovirus (HCMV), hepatitis B virus (HBV), hepatitis C virus (HCV), human herpes virus (HHV), human mammary tumor virus (HMTV), human papillomavirus 16 (HPV16), human papillomavirus 18 (HPV18), human papillomavirus 60 (HPV-60), human papillomavirus ZM130 (HPV8-ZM130), human T-cell leukemia virus type 1 (HTLV-1), John Cunningham virus (JCV), molluscum contagiosum virus (MCV), and simian vacuolating virus 40 (SV40)

170. The method of any one of claims 146-166, wherein the set of pathogens comprises any combination of human herpes virus 5 CINCY-TOWNE (HHV5-CINCY-TOWNE) virus, Epstein-Barr B95-8 (EBV-B95-8 virus), molluscum contagiosum virus R17b (MCV-R17b) virus, human papillomavirus 16 (HPV16) virus, human cytomegalovirus AD169 (HCMV-AD169) virus, hepatitis B virus (HBV) virus, hepatitis B virus 18 (HPV18) virus, hepatitis C virus (HCV) virus, human papillomavirus 8-ZM130 (HPV8-ZM130) virus, and John Cunningham virus PLYCG (JCV-PLYCG) virus.

171. A method of screening for a cancer condition in a test subject, the method comprising:
(a) obtaining a first biological sample from the test subject, wherein the first biological sample comprises cell-free nucleic acid from the test subject and potentially cell-free nucleic acid from at least one pathogen in a set of pathogens;
(b) sequencing the cell-free nucleic acid in the first biological sample to generate a plurality of sequence reads from the test subject;
(c) evaluating the plurality of sequence reads to obtain an indication as to whether a methylation signature associated with a respective pathogen in the set of pathogens is present or absent; and
(d) using the indication as to whether the methylation signature associated with the respective pathogen is present or absent to determine whether the test subject has the cancer condition or the likelihood that test subject has the cancer condition.

172. The method of claim 171, wherein the method further comprises:
evaluating the plurality of sequence reads to obtain an indication as to whether an APOBEC induced mutational signature associated with a first pathogen in the set of pathogens is present or absent; and wherein
the using (d) uses the indication as to whether the APOBEC induced mutational signature associated with the first pathogen is present or absent along with the indication as to whether the methylation signature associated with the respective pathogen is present or absent to determine whether the test subject has the cancer condition or the likelihood that the test subject has the cancer condition.

173. The method of claim 171, wherein the method further comprises:
evaluating, via k-mer analysis, the plurality of sequence reads to obtain an indication as to whether an APOBEC induced mutational signature is present or absent; and wherein
the using (d) uses the indication as to whether the APOBEC induced mutational signature associated with the first pathogen is present or absent along with the indication as to whether the methylation signature associated with the respective pathogen is present or absent to determine whether the test subject has the cancer condition or the likelihood that test subject has the cancer condition.

174. The method of any one of claims 172-173, wherein
the indication as to whether the APOBEC induced mutational signature associated with the first pathogen is present or absent further includes a measure of enrichment of the APOBEC induced mutational signature; and
the using (d) uses the measure of enrichment of the APOBEC induced mutational signature along with the indication as to whether the methylation signature associated with the respective pathogen is present or absent to determine whether the test subject has the cancer condition or the likelihood that the test subject has the cancer condition.

175. The method of any one of claims 171-174, wherein the method further comprises:
analyzing the first biological sample or a second biological sample from the test subject for an expression of an APOBEC protein associated with a first pathogen in the set of pathogens, and wherein
the using (d) uses the expression of the APOBEC protein along with the indication as to whether the methylation signature associated with the respective pathogen is present or absent to determine whether the test subject has the cancer condition or the likelihood that the test subject has the cancer condition.

176. The method of claim 175, wherein the APOBEC protein is APOBEC1, APOBEC2, APOBEC3A, APOBEC3B, APOBEC3C, APOBEC3D, APOBEC3F, APOBEC3G, APOBEC3H, or APOBEC4.

177. The method of any one of claims 171-176, the method further comprising:
performing an assay comprising measuring an amount of an APOBEC induced mutational signature of the cell-free nucleic acid in the first biological sample; and wherein
the using (d) uses the indication as to whether the APOBEC induced mutational signature associated with the first pathogen is present or absent along with the indication as to whether the methylation signature associated with the respective pathogen is present or absent to determine whether the test subject has the cancer condition or the likelihood that the test subject has the cancer condition.

178. The method of claim 171, the method further comprising:
obtaining a second biological sample from the test subject, wherein the second biological sample comprises cell-free nucleic acid from the test subject and potentially cell-free nucleic acid from a first pathogen in the set of pathogens; and
performing an assay comprising measuring an amount of an APOBEC induced mutational signature of the cell-free nucleic acid in the second biological sample; and wherein
the using (d) uses the indication as to whether the APOBEC induced mutational signature associated with the first pathogen is present or absent along with the indication as to whether the methylation signature associated with the respective pathogen is present or absent to determine whether the test subject has the cancer condition or the likelihood that the test subject has the cancer condition.

179. The method of any one of claims 172-178, wherein the APOBEC induced mutational signature is selected from either mutation signature type 2 or mutation signature type 13.

180. The method of claim 171, wherein the sequencing (b) is performed by whole genome sequencing, targeted panel sequencing, or whole genome bisulfite sequencing.

181. The method of claim 171, wherein the test subject is human.

182. The method of claim 181, wherein the cancer condition is cervical cancer, hepatocellular carcinoma, bladder cancer, breast cancer, esophageal cancer, prostate cancer, nasopharyngeal cancer, lung cancer, lymphoma, or leukemia.

183. The method of claim 182, wherein the cancer condition is early stage cancer.

184. The method of claim 181, wherein the cancer condition is renal cancer, hepatocellular carcinoma, colorectal cancer, esophageal cancer, breast cancer, lung cancer, nasopharyngeal cancer, thyroid cancer, lymphoma, ovarian cancer, or cervical cancer.

185. The method of claim 184, wherein the cancer condition is late stage cancer.

186. The method of claim 171, wherein the cancer condition is a liquid cancer, a liver cancer, or lung cancer.

187. The method of any one of claims 171-186, wherein the first biological sample and the second biological sample are plasma.

188. The method of any one of claims 171-186, wherein the first biological sample and the second biological sample are different aliquots of the same biological sample from the test subject.

189. The method of any one of claims 171-186, wherein the first biological sample and the second biological sample are the same biological sample.

190. The method of any one of claims 171-186, wherein the first biological sample or the second biological sample comprises blood, whole blood, plasma, serum, urine, cerebrospinal fluid, fecal, saliva, sweat, tears, pleural fluid, pericardial fluid, or peritoneal fluid of the test subject.

191. The method of any one of claims 171-186, wherein the first biological sample or the second biological sample consists of blood, whole blood, plasma, serum, urine, cerebrospinal fluid, fecal, saliva, sweat, tears, pleural fluid, pericardial fluid, or peritoneal fluid of the test subject.

192. The method of any one of claims 171-190, wherein a respective pathogen in the set of pathogens is Epstein-Barr virus (EBV), human cytomegalovirus (HCMV), hepatitis B virus (HBV), hepatitis C virus (HCV), human herpes virus (HHV), human mammary tumor virus (HMTV), human papillomavirus 16 (HPV16), human papillomavirus 18 (HPV18), human papillomavirus 60 (HPV-60), human papillomavirus ZM130 (HPV8-ZM130), human T-cell leukemia virus type 1 (HTLV-1), John Cunningham virus (JCV), molluscum contagiosum virus (MCV), or simian vacuolating virus 40 (SV40).

193. The method of any one of claims 171-190, wherein the set of pathogens is all or a subset of the RefSeq viral genome database.

194. The method of any one of claims 171-190, wherein the set of pathogens comprises any combination of the Epstein-Barr virus (EBV), human cytomegalovirus (HCMV), hepatitis B virus (HBV), hepatitis C virus (HCV), human herpes virus (HHV), human mammary tumor virus (HMTV), human papillomavirus 16 (HPV16), human papillomavirus 18 (HPV18), human papillomavirus 60 (HPV-60), human papillomavirus ZM130 (HPV8-ZM130), human T-cell leukemia virus type 1 (HTLV-1), John Cunningham virus (JCV), molluscum contagiosum virus (MCV), and simian vacuolating virus 40 (SV40).

195. The method of any one of claims 171-190, wherein the set of pathogens comprises any combination of human herpes virus 5 CINCY-TOWNE (HHV5-CINCY-TOWNE) virus, Epstein-Barr B95-8 (EBV-B95-8 virus), molluscum contagiosum virus R17b (MCV-R17b) virus, human papillomavirus 16 (HPV16) virus, human cytomegalovirus AD169 (HCMV-AD169) virus, hepatitis B virus (HBV) virus, hepatitis B virus 18 (HPV18) virus, hepatitis C virus (HCV) virus, human papillomavirus 8-ZM130 (HPV8-ZM130) virus, and John Cunningham virus PLYCG (JCV-PLYCG) virus.

196. A method of screening for a cancer condition in a test subject, the method comprising:
(a) obtaining a first biological sample from the test subject, wherein the first biological sample comprises cell-free nucleic acid from the test subject and potentially cell-free nucleic acid from at least one pathogen in a set of pathogens;
(b) sequencing the cell-free nucleic acid in the first biological sample to generate a plurality of sequence reads from the test subject;
(c) evaluating the plurality of sequence reads to obtain an indication as to whether a sequence fragment signature associated with a respective pathogen in the set of pathogens is present or absent;
(d) evaluating the plurality of sequence reads to obtain an indication as to whether a methylation signature associated with a respective pathogen in the set of pathogens is present or absent; and
(e) using the indication as to whether the signature fragment signature associated with a respective pathogen is present or absent and the indication as to whether the methylation signature associated with a respective pathogen is present or absent to determine whether the test subject has the cancer condition or the likelihood that test subject has the cancer condition.

197. The method of claim 196, wherein the method further comprises:
evaluating the plurality of sequence reads to obtain an indication as to whether an APOBEC induced mutational signature associated with a first pathogen in the set of pathogens is present or absent; and wherein
the using (e) comprises using (i) the indication as to whether the signature fragment signature associated with a respective pathogen is present or absent, (ii) the indication as to whether the methylation signature associated with a respective pathogen is present or absent, and (iii) the indication as to whether an APOBEC induced mutational signature associated with a first pathogen in the set of pathogens to determine whether the test subject has the cancer condition or the likelihood that the test subject has the cancer condition.

198. The method of claim 196, wherein the method further comprises:
evaluating, via k-mer analysis, the plurality of sequence reads to obtain an indication as to whether an APOBEC induced mutational signature is present or absent; and wherein
the using (e) comprises using (i) the indication as to whether the signature fragment signature associated with a respective pathogen is present or absent, (ii) the indication as to whether the methylation signature associated with a respective pathogen is present or absent, and (iii) the indication as to whether an APOBEC induced mutational signature associated with a first pathogen in the set of pathogens to determine whether the test subject has the cancer condition or the likelihood that the test subject has the cancer condition.

199. The method of any one of claims 197-198, wherein
the indication as to whether the APOBEC induced mutational signature associated with the first pathogen is present or absent further includes a measure of enrichment of the APOBEC induced mutational signature; and
the using (e) comprises using (i) the indication as to whether the signature fragment signature associated with a respective pathogen is present or absent, (ii) the indication as to whether the methylation signature associated with a respective pathogen is present or absent, and (iii) the measure of enrichment of the APOBEC induced mutational signature to determine whether the test subject has the cancer condition or the likelihood that the test subject has the cancer condition.

200. The method of any one of claims 196-199, wherein the method further comprises:
analyzing the first biological sample or a second biological sample from the test subject for an expression of an APOBEC protein associated with a first pathogen in the set of pathogens, and wherein
the using (e) comprises using (i) the indication as to whether the signature fragment signature associated with a respective pathogen is present or absent, (ii) the indication as to whether the methylation signature associated with a respective pathogen is present or absent, and (iii) the expression of an APOBEC protein associated with a first pathogen in the set of pathogens to determine whether the test subject has the cancer condition or the likelihood that the test subject has the cancer condition.

201. The method of claim 200, wherein the APOBEC protein is APOBEC1, APOBEC2, APOBEC3A, APOBEC3B, APOBEC3C, APOBEC3D, APOBEC3F, APOBEC3G, APOBEC3H, or APOBEC4.

202. The method of any one of claims 196-201, the method further comprising:
performing an assay comprising measuring an amount of an APOBEC induced mutational signature of the cell-free nucleic acid in the first biological sample; and wherein
the using (e) comprises using (i) the indication as to whether the signature fragment signature associated with a respective pathogen is present or absent, (ii) the indication as to whether the methylation signature associated with a respective pathogen is present or absent, and (iii) the amount of the APOBEC induced mutational signature and the set of amounts of sequence reads to determine whether the test subject has the cancer condition or the likelihood that the test subject has the cancer condition.

203. The method of claim 196, the method further comprising:
obtaining a second biological sample from the test subject, wherein the second biological sample comprises cell-free nucleic acid from the test subject and potentially cell-free nucleic acid from a first pathogen in the set of pathogens; and
performing an assay comprising measuring an amount of an APOBEC induced mutational signature of the cell-free nucleic acid in the second biological sample; and wherein
the using (e) comprises using (i) the indication as to whether the signature fragment signature associated with a respective pathogen is present or absent, (ii) the indication as to whether the methylation signature associated with a respective pathogen is present or absent, and (iii) the amount of the APOBEC induced mutational signature and the set of amounts of sequence reads to determine whether the test subject has the cancer condition or the likelihood that the test subject has the cancer condition.

204. The method of any one of claims 197-203, wherein the APOBEC induced mutational signature is selected from either mutation signature type 2 or mutation signature type 13.

205. The method of claim 196, wherein the sequencing (b) is performed by whole genome sequencing, targeted panel sequencing, or whole genome bisulfite sequencing.

206. The method of claim 196, wherein the test subject is human.

207. The method of claim 206, wherein the cancer condition is cervical cancer, hepatocellular carcinoma, bladder cancer, breast cancer, esophageal cancer, prostate cancer, nasopharyngeal cancer, lung cancer, lymphoma, or leukemia.

208. The method of claim 196, wherein the cancer condition is early stage cancer.

209. The method of claim 206, wherein the cancer condition is renal cancer, hepatocellular carcinoma, colorectal cancer, esophageal cancer, breast cancer, lung cancer, nasopharyngeal cancer, thyroid cancer, lymphoma, ovarian cancer, or cervical cancer.

210. The method of claim 209, wherein the cancer condition is late stage cancer.

211. The method of claim 196, wherein the cancer condition is a liquid cancer, a liver cancer, or lung cancer.

212. The method of any one of claims 196-211, wherein the first biological sample and the second biological sample are plasma.

213. The method of any one of claims 196-211, wherein the first biological sample and the second biological sample are different aliquots of the same biological sample from the test subject.

214. The method of any one of claims 196-211, wherein the first biological sample and the second biological sample are the same biological sample.

215. The method of any one of claims 196-211, wherein the first biological sample or the second biological sample comprises blood, whole blood, plasma, serum, urine, cerebrospinal fluid, fecal, saliva, sweat, tears, pleural fluid, pericardial fluid, or peritoneal fluid of the test subject.

216. The method of any one of claims 196-211, wherein the first biological sample or the second biological sample consists of blood, whole blood, plasma, serum, urine, cerebrospinal fluid, fecal, saliva, sweat, tears, pleural fluid, pericardial fluid, or peritoneal fluid of the test subject.

217. The method of any one of claims 196-216, wherein a respective pathogen in the set of pathogens is Epstein-Barr virus (EBV), human cytomegalovirus (HCMV), hepatitis B virus (HBV), hepatitis C virus (HCV), human herpes virus (HHV), human mammary tumor virus (HMTV), human papillomavirus 16 (HPV16), human papillomavirus 18 (HPV18), human papillomavirus 60 (HPV-60), human papillomavirus ZM130 (HPV8-ZM130), human T-cell leukemia virus type 1 (HTLV-1), John Cunningham virus (JCV), molluscum contagiosum virus (MCV), or simian vacuolating virus 40 (SV40).

218. The method of any one of claims 196-216, wherein the set of pathogens is all or a subset of the RefSeq viral genome database.

219. The method of any one of claims 196-216, wherein the set of pathogens comprises any combination of the Epstein-Barr virus (EBV), human cytomegalovirus (HCMV), hepatitis B virus (HBV), hepatitis C virus (HCV), human herpes virus (HHV), human mammary tumor virus (HMTV), human papillomavirus 16 (HPV16), human papillomavirus 18 (HPV18), human papillomavirus 60 (HPV-60), human papillomavirus ZM130 (HPV8-ZM130), human T-cell leukemia virus type 1 (HTLV-1), John Cunningham virus (JCV), molluscum contagiosum virus (MCV), and simian vacuolating virus 40 (SV40).

220. The method of any one of claims 196-216, wherein the set of pathogens comprises any combination of human herpes virus 5 CINCY-TOWNE (HHV5-CINCY-TOWNE) virus, Epstein-Barr B95-8 (EBV-B95-8 virus), molluscum contagiosum virus R17b (MCV-R17b) virus, human papillomavirus 16 (HPV16) virus, human cytomegalovirus AD169 (HCMV-AD169) virus, hepatitis B virus (HBV) virus, hepatitis B virus 18 (HPV18) virus, hepatitis C virus (HCV) virus, human papillomavirus 8-ZM130 (HPV8-ZM130) virus, and John Cunningham virus PLYCG (JCV-PLYCG) virus.

221. A method of screening for a cancer condition in a test subject, the method comprising:
(a) obtaining a first biological sample from the test subject, wherein the first biological sample comprises cell-free nucleic acid from the test subject and potentially cell-free nucleic acid from a first pathogen in a set of pathogens; and
(b) performing an assay comprising:
i. sequencing of the cell-free nucleic acid in the biological sample to generate a plurality of sequence reads from the test subject,
ii. determining an amount of the plurality of sequence reads that align to a reference genome of the first pathogen, and
iii. thresholding the amount on an amount of sequence reads associated with a predetermined percentile of a first distribution, wherein each respective subject in a cohort of subjects that do not have the cancer condition contributes to the first distribution an amount of sequence reads from the respective subject that align to the reference genome of the first pathogen, thereby determining a scaled first amount of the plurality of sequence reads from the test subject; and
wherein the test subject is deemed to have the cancer condition when a metric based, at least in part, on the scaled first amount of the plurality of sequence reads satisfies a threshold associated with the cancer condition.

222. The method of claim 221, wherein the method further comprises:
evaluating the plurality of sequence reads to obtain an indication as to whether an APOBEC induced mutational signature associated with a first pathogen in the set of pathogens is present or absent; and wherein
the test subject is deemed to have the cancer condition when a metric, based on the APOBEC induced mutational signature associated with the first pathogen is present or absent and the scaled first amount of the plurality of sequence reads, satisfies a threshold associated with the cancer condition.

223. The method of claim 221, wherein the method further comprises:
evaluating, via k-mer analysis, the plurality of sequence reads to obtain an indication as to whether an APOBEC induced mutational signature is present or absent; and wherein
the test subject is deemed to have the cancer condition when a metric, based on the APOBEC induced mutational signature associated with the first pathogen is present or absent and the scaled first amount of the plurality of sequence reads, satisfies a threshold associated with the cancer condition.

224. The method of any one of claims 222-223, wherein
the indication as to whether the APOBEC induced mutational signature associated with the first pathogen is present or absent further includes a measure of enrichment of the APOBEC induced mutational signature; and wherein
the test subject is deemed to have the cancer condition when a metric, based on the measure of enrichment of the APOBEC induced mutational signature and the scaled first amount of the plurality of sequence reads, satisfies a threshold associated with the cancer condition.

225. The method of any one of claims 221-224, wherein the method further comprises:
analyzing the first biological sample or a second biological sample from the test subject for an expression of an APOBEC protein associated with a first pathogen in the set of pathogens, and wherein
the test subject is deemed to have the cancer condition when a metric, based on the expression of an APOBEC protein associated with a first pathogen in the set of pathogens and the scaled first amount of the plurality of sequence reads, satisfies a threshold associated with the cancer condition.

226. The method of claim 225, wherein the APOBEC protein is APOBEC1, APOBEC2, APOBEC3A, APOBEC3B, APOBEC3C, APOBEC3D, APOBEC3F, APOBEC3G, APOBEC3H, or APOBEC4.

227. The method of any one of claims 221-226, the method further comprising:
performing an assay comprising measuring an amount of an APOBEC induced mutational signature of the cell-free nucleic acid in the first biological sample; and wherein
the test subject is deemed to have the cancer condition when a metric, based on the amount of an APOBEC induced mutational signature and the scaled first amount of the plurality of sequence reads, satisfies a threshold associated with the cancer condition.

228. The method of claim 221, the method further comprising:
obtaining a second biological sample from the test subject, wherein the second biological sample comprises cell-free nucleic acid from the test subject and potentially cell-free nucleic acid from a first pathogen in the set of pathogens; and
performing an assay comprising measuring an amount of an APOBEC induced mutational signature of the cell-free nucleic acid in the second biological sample; and wherein
the test subject is deemed to have the cancer condition when a metric, based on the amount of an APOBEC induced mutational signature and the scaled first amount of the plurality of sequence reads, satisfies a threshold associated with the cancer condition.

229. The method of any one of claims 222-228, wherein the APOBEC induced mutational signature is selected from either mutation signature type 2 or mutation signature type 13.

230. The method of claim 221, wherein the sequencing (b)(i) is performed by whole genome sequencing, targeted panel sequencing, or whole genome bisulfite sequencing.

231. The method of claim 221, wherein the test subject is deemed by the assay to have the cancer condition when the scaled first amount of the plurality of sequence reads from the test subject exceeds the amount of sequence reads associated with the predetermined percentile of the distribution by a predetermined cutoff value.

232. The method of claim 231, wherein the first predetermined cutoff value is a single standard deviation greater than a measure of central tendency of the distribution.

233. The method of claim 231, wherein the first predetermined cutoff value is three standard deviations greater than a measure of central tendency of the distribution.

234. The method of claim 221, wherein the test subject is human.

235. The method of claim 234, wherein the cancer condition is cervical cancer, hepatocellular carcinoma, bladder cancer, breast cancer, esophageal cancer, prostate cancer, nasopharyngeal cancer, lung cancer, lymphoma, or leukemia.

236. The method of claim 235, wherein the cancer condition is early stage cancer.

237. The method of claim 221, wherein the cancer condition is renal cancer, hepatocellular carcinoma, colorectal cancer, esophageal cancer, breast cancer, lung cancer, nasopharyngeal cancer, thyroid cancer, lymphoma, ovarian cancer, or cervical cancer.

238. The method of claim 237, wherein the cancer condition is late stage cancer.

239. The method of claim 221, wherein the cancer condition is a liquid cancer, a liver cancer, or lung cancer.

240. The method of any one of claims 221-239, wherein the first biological sample and the second biological sample are plasma.

241. The method of any one of claims 221-239, wherein the first biological sample and the second biological sample are different aliquots of the same biological sample from the test subject.

242. The method of any one of claims 221-239, wherein the first biological sample and the second biological sample are the same biological sample.

243. The method of any one of claims 221-239, wherein the first biological sample or the second biological sample comprises blood, whole blood, plasma, serum, urine, cerebrospinal fluid, fecal, saliva, sweat, tears, pleural fluid, pericardial fluid, or peritoneal fluid of the test subject.

244. The method of any one of claims 221-239, wherein the first biological sample or the second biological sample consists of blood, whole blood, plasma, serum, urine, cerebrospinal fluid, fecal, saliva, sweat, tears, pleural fluid, pericardial fluid, or peritoneal fluid of the test subject.

245. The method of any one of claims 221-242, wherein a respective pathogen in the set of pathogens is Epstein-Barr virus (EBV), human cytomegalovirus (HCMV), hepatitis B virus (HBV), hepatitis C virus (HCV), human herpes virus (HHV), human mammary tumor virus (HMTV), human papillomavirus 16 (HPV16), human papillomavirus 18 (HPV18), human papillomavirus 60 (HPV-60), human papillomavirus ZM130 (HPV8-ZM130), human T-cell leukemia virus type 1 (HTLV-1), John Cunningham virus (JCV), molluscum contagiosum virus (MCV), or simian vacuolating virus 40 (SV40).

246. The method of any one of claims 221-242, wherein the set of pathogens is all or a subset of the RefSeq viral genome database.

247. The method of any one of claims 221-242, wherein the set of pathogens comprises any combination of the Epstein-Barr virus (EBV), human cytomegalovirus (HCMV), hepatitis B virus (HBV), hepatitis C virus (HCV), human herpes virus (HHV), human mammary tumor virus (HMTV), human papillomavirus 16 (HPV16), human papillomavirus 18 (HPV18), human papillomavirus 60 (HPV-60), human papillomavirus ZM130 (HPV8-ZM130), human T-cell leukemia virus type 1 (HTLV-1), John Cunningham virus (JCV), molluscum contagiosum virus (MCV), and simian vacuolating virus 40 (SV40).

248. The method of any one of claims 221-242, wherein the set of pathogens comprises any combination of human herpes virus 5 CINCY-TOWNE (HHV5-CINCY-TOWNE) virus, Epstein-Barr B95-8 (EBV-B95-8 virus), molluscum contagiosum virus R17b (MCV-R17b) virus, human papillomavirus 16 (HPV16) virus, human cytomegalovirus AD169 (HCMV-AD169) virus, hepatitis B virus (HBV) virus, hepatitis B virus 18 (HPV18) virus, hepatitis C virus (HCV) virus, human papillomavirus 8-ZM130 (HPV8-ZM130) virus, and John Cunningham virus PLYCG (JCV-PLYCG) virus.

249. A method of screening for each cancer condition in a plurality of cancer conditions in a test subject, the method comprising:
(a) obtaining a first biological sample from the test subject, wherein the first biological sample comprises cell-free nucleic acid from the test subject and potentially cell-free nucleic acid from any pathogen in a set of pathogens;
(b) sequencing of the cell-free nucleic acid in the biological sample to generate a plurality of sequence reads from the test subject;
(c) performing a procedure, for each respective pathogen in the set of pathogens, the procedure comprising:
i. determining a respective amount of the plurality of sequence reads that align to a reference genome of the respective pathogen, and
ii. thresholding the respective amount on an amount of sequence reads associated with a predetermined percentile of a respective distribution, wherein each respective subject in a respective cohort of subjects that do not have a cancer condition in the plurality of cancer conditions contributes to the respective distribution an amount of sequence reads from the respective subject that align to the reference genome of the respective pathogen, thereby determining a scaled respective amount of the plurality of sequence reads from the respective subject; and
(d) inputting at least each scaled respective amount of the plurality of sequence reads into a first classifier, thereby obtaining a classifier result that indicates whether the test has a cancer condition in the plurality of cancer conditions.

250. The method of claim 249, wherein the method further comprises:
evaluating the plurality of sequence reads to obtain an indication as to whether an APOBEC induced mutational signature associated with a first pathogen in the set of pathogens is present or absent; and wherein
the inputting (d) inputs the indication as to whether the APOBEC induced mutational signature associated with the first pathogen is present or absent along with each scaled respective amount of the plurality of sequence reads into the first classifier, thereby obtaining a classifier result that indicates whether the test has a cancer condition in the plurality of cancer conditions.

251. The method of claim 249, wherein the method further comprises:
evaluating, via k-mer analysis, the plurality of sequence reads to obtain an indication as to whether an APOBEC induced mutational signature is present or absent; and wherein
the inputting (d) inputs the indication as to whether the APOBEC induced mutational signature associated with the first pathogen is present or absent along with each scaled respective amount of the plurality of sequence reads into the first classifier, thereby obtaining a classifier result that indicates whether the test has a cancer condition in the plurality of cancer conditions.

252. The method of any one of claims 250-251, wherein
the indication as to whether the APOBEC induced mutational signature associated with the first pathogen is present or absent further includes a measure of enrichment of the APOBEC induced mutational signature; and
the inputting (d) inputs the measure of enrichment of the APOBEC induced mutational signature along with each scaled respective amount of the plurality of sequence reads into the first classifier, thereby obtaining a classifier result that indicates whether the test has a cancer condition in the plurality of cancer conditions.

253. The method of any one of claims 249-252, wherein the method further comprises:
analyzing the first biological sample or a second biological sample from the test subject for an expression of an APOBEC protein associated with a first pathogen in the set of pathogens, and wherein
the inputting (d) inputs the expression of the APOBEC protein along with each scaled respective amount of the plurality of sequence reads into the first classifier, thereby obtaining a classifier result that indicates whether the test has a cancer condition in the plurality of cancer conditions.

254. The method of claim 253, wherein the APOBEC protein is APOBEC1, APOBEC2, APOBEC3A, APOBEC3B, APOBEC3C, APOBEC3D, APOBEC3F, APOBEC3G, APOBEC3H, or APOBEC4.

255. The method of any one of claims 249-254, the method further comprising:
performing an assay comprising measuring an amount of an APOBEC induced mutational signature of the cell-free nucleic acid in the first biological sample; and wherein
the inputting (d) inputs the amount of an APOBEC induced mutational signature along with each scaled respective amount of the plurality of sequence reads into the first classifier, thereby obtaining a classifier result that indicates whether the test has a cancer condition in the plurality of cancer conditions.

256. The method of claim 249, the method further comprising:
obtaining a second biological sample from the test subject, wherein the second biological sample comprises cell-free nucleic acid from the test subject and potentially cell-free nucleic acid from a first pathogen in the set of pathogens; and
performing an assay comprising measuring an amount of an APOBEC induced mutational signature of the cell-free nucleic acid in the second biological sample; and wherein
the inputting (d) inputs the amount of an APOBEC induced mutational signature along with each scaled respective amount of the plurality of sequence reads into the first classifier, thereby obtaining a classifier result that indicates whether the test has a cancer condition in the plurality of cancer conditions.

257. The method of any one of claims 250-256, wherein the APOBEC induced mutational signature is selected from either mutation signature type 2 or mutation signature type 13.

258. The method of claim 249, wherein the sequencing (b) is performed by whole genome sequencing, targeted panel sequencing, or whole genome bisulfite sequencing.

259. The method of claim 249, wherein the test subject is human.

260. The method of claim 258, wherein a cancer condition in the plurality of cancer conditions is cervical cancer, hepatocellular carcinoma, bladder cancer, breast cancer, esophageal cancer, prostate cancer, nasopharyngeal cancer, lung cancer, lymphoma, or leukemia.

261. The method of claim 260, wherein the cancer condition is early stage cancer.

262. The method of claim 258, wherein a cancer condition in the plurality of cancer conditions is renal cancer, hepatocellular carcinoma, colorectal cancer, esophageal cancer, breast cancer, lung cancer, nasopharyngeal cancer, thyroid cancer, lymphoma, ovarian cancer, or cervical cancer.

263. The method of claim 262, wherein the cancer condition is late stage cancer.

264. The method of claim 260, wherein a cancer condition in the plurality of cancer conditions is a liquid cancer, a liver cancer, or lung cancer.

265. The method of any one of claims 249-264, wherein the first biological sample and the second biological sample are plasma.

266. The method of any one of claims 249-264, wherein the first biological sample and the second biological sample are different aliquots of the same biological sample from the test subject.

267. The method of any one of claims 249-264, wherein the first biological sample and the second biological sample are the same biological sample.

268. The method of any one of claims 249-264, wherein the first biological sample or the second biological sample comprises blood, whole blood, plasma, serum, urine, cerebrospinal fluid, fecal, saliva, sweat, tears, pleural fluid, pericardial fluid, or peritoneal fluid of the test subject.

269. The method of any one of claims 249-264, wherein the first biological sample or the second biological sample consists of blood, whole blood, plasma, serum, urine, cerebrospinal fluid, fecal, saliva, sweat, tears, pleural fluid, pericardial fluid, or peritoneal fluid of the test subject.

270. The method of any one of claims 249-269, wherein a respective pathogen in the set of pathogens is Epstein-Barr virus (EBV), human cytomegalovirus (HCMV), hepatitis B virus (HBV), hepatitis C virus (HCV), human herpes virus (HHV), human mammary tumor virus (HMTV), human papillomavirus 16 (HPV16), human papillomavirus 18 (HPV18), human papillomavirus 60 (HPV-60), human papillomavirus ZM130 (HPV8-ZM130), human T-cell leukemia virus type 1 (HTLV-1), John Cunningham virus (JCV), molluscum contagiosum virus (MCV), or simian vacuolating virus 40 (SV40).

271. The method of any one of claims 249-269, wherein the set of pathogens is all or a subset of the RefSeq viral genome database.

272. The method of any one of claims 249-269, wherein the set of pathogens comprises any combination of the Epstein-Barr virus (EBV), human cytomegalovirus (HCMV), hepatitis B virus (HBV), hepatitis C virus (HCV), human herpes virus (HHV), human mammary tumor virus (HMTV), human papillomavirus 16 (HPV16), human papillomavirus 18 (HPV18), human papillomavirus 60 (HPV-60), human papillomavirus ZM130 (HPV8-ZM130), human T-cell leukemia virus type 1 (HTLV-1), John Cunningham virus (JCV), molluscum contagiosum virus (MCV), and simian vacuolating virus 40 (SV40).

273. The method of any one of claims 249-269, wherein the set of pathogens comprises any combination of human herpes virus 5 CINCY-TOWNE (HHV5-CINCY-TOWNE) virus, Epstein-Barr B95-8 (EBV-B95-8 virus), molluscum contagiosum virus R17b (MCV-R17b) virus, human papillomavirus 16 (HPV16) virus, human cytomegalovirus AD169 (HCMV-AD169) virus, hepatitis B virus (HBV) virus, hepatitis B virus 18 (HPV18) virus, hepatitis C virus (HCV) virus, human papillomavirus 8-ZM130 (HPV8-ZM130) virus, and John Cunningham virus PLYCG (JCV-PLYCG) virus.

274. The method of any one of claims 249-269, wherein the set of pathogens comprises at least two pathogens.

275. The method of any one of claims 249-269, wherein the set of pathogens comprises at least twenty pathogens.

276. A method of screening for each cancer condition in a plurality of cancer conditions in a test subject, the method comprising:
(a) obtaining a first biological sample from the test subject, wherein the biological sample comprises cell-free nucleic acid from the test subject and potentially cell-free nucleic acid from any pathogen in a set of pathogens;
(b) sequencing of the cell-free nucleic acid in the biological sample to generate a plurality of sequence reads from the test subject;
(c) performing a procedure, for each respective pathogen in the set of pathogens, the procedure comprising:
i. determining a respective amount of the plurality of sequence reads that align to a reference genome of the respective pathogen, and
ii. thresholding the respective amount on an amount of sequence reads associated with a predetermined percentile of a respective distribution, wherein each respective subject in a respective cohort of subjects that do not have a cancer condition in the plurality of cancer conditions contributes to the respective distribution an amount of sequence reads from the respective subject that align to the reference genome of the respective pathogen, thereby determining a scaled respective amount of the plurality of sequence reads from the respective subject; and
(d) inputting at least each scaled respective amount of the plurality of sequence reads into each classifier in a plurality of classifiers, wherein each classifier in the plurality of classifier indicates whether the respective subject has or does not have a corresponding single cancer condition in the plurality of cancer conditions.

277. The method of claim 276, wherein the method further comprises:
evaluating the plurality of sequence reads to obtain an indication as to whether an APOBEC induced mutational signature associated with a first pathogen in the set of pathogens is present or absent; and wherein
the inputting (d) inputs the indication as to whether the APOBEC induced mutational signature associated with the first pathogen is present or absent along with each scaled respective amount of the plurality of sequence reads into each classifier in the plurality of classifiers, wherein each classifier in the plurality of classifier indicates whether the respective subject has or does not have a corresponding single cancer condition in the plurality of cancer conditions.

278. The method of claim 276, wherein the method further comprises:
evaluating, via k-mer analysis, the plurality of sequence reads to obtain an indication as to whether an APOBEC induced mutational signature is present or absent; and wherein
the inputting (d) inputs the indication as to whether the APOBEC induced mutational signature associated with the first pathogen is present or absent along with each scaled respective amount of the plurality of sequence reads into each classifier in the plurality of classifiers, wherein each classifier in the plurality of classifier indicates whether the respective subject has or does not have a corresponding single cancer condition in the plurality of cancer conditions.

279. The method of any one of claims 277-278, wherein
the indication as to whether the APOBEC induced mutational signature associated with the first pathogen is present or absent further includes a measure of enrichment of the APOBEC induced mutational signature; and
the inputting (d) inputs the measure of enrichment of the APOBEC induced mutational signature along with each scaled respective amount of the plurality of sequence reads into each classifier in a plurality of classifiers, wherein each classifier in the plurality of classifier indicates whether the respective subject has or does not have a corresponding single cancer condition in the plurality of cancer conditions.

280. The method of any one of claims 276-279, wherein the method further comprises:
analyzing the first biological sample or a second biological sample from the test subject for an expression of an APOBEC protein associated with a first pathogen in the set of pathogens, and wherein
the inputting (d) inputs the expression of the APOBEC protein along with each scaled respective amount of the plurality of sequence reads into each classifier in the plurality of classifiers, wherein each classifier in the plurality of classifier indicates whether the respective subject has or does not have a corresponding single cancer condition in the plurality of cancer conditions.

281. The method of claim 280, wherein the APOBEC protein is APOBEC1, APOBEC2, APOBEC3A, APOBEC3B, APOBEC3C, APOBEC3D, APOBEC3F, APOBEC3G, APOBEC3H, or APOBEC4.

282. The method of any one of claims 276-281, the method further comprising:
performing an assay comprising measuring an amount of an APOBEC induced mutational signature of the cell-free nucleic acid in the first biological sample; and wherein
the inputting (d) inputs the amount of an APOBEC induced mutational signature along with each scaled respective amount of the plurality of sequence reads into each classifier in the plurality of classifiers, wherein each classifier in the plurality of classifier indicates whether the respective subject has or does not have a corresponding single cancer condition in the plurality of cancer conditions.

283. The method of claim 276, the method further comprising:
obtaining a second biological sample from the test subject, wherein the second biological sample comprises cell-free nucleic acid from the test subject and potentially cell-free nucleic acid from a first pathogen in the set of pathogens; and
performing an assay comprising measuring an amount of an APOBEC induced mutational signature of the cell-free nucleic acid in the second biological sample; and wherein
the inputting (d) inputs the amount of an APOBEC induced mutational signature along with each scaled respective amount of the plurality of sequence reads into each classifier in the plurality of classifiers, wherein each classifier in the plurality of classifier indicates whether the respective subject has or does not have a corresponding single cancer condition in the plurality of cancer conditions.

284. The method of any one of claims 277-283, wherein the APOBEC induced mutational signature is selected from either mutation signature type 2 or mutation signature type 13.

285. The method of claim 276, wherein the sequencing (b) is performed by whole genome sequencing, targeted panel sequencing, or whole genome bisulfite sequencing.

286. The method of claim 276, wherein the test subject is human.

287. The method of claim 285, wherein a cancer condition in the plurality of cancer conditions is cervical cancer, hepatocellular carcinoma, bladder cancer, breast cancer, esophageal cancer, prostate cancer, nasopharyngeal cancer, lung cancer, lymphoma, or leukemia.

288. The method of claim 287, wherein the cancer condition is early stage cancer.

289. The method of claim 285, wherein a cancer condition in the plurality of cancer conditions is renal cancer, hepatocellular carcinoma, colorectal cancer, esophageal cancer, breast cancer, lung cancer, nasopharyngeal cancer, thyroid cancer, lymphoma, ovarian cancer, or cervical cancer.

290. The method of claim 289, wherein the cancer condition is late stage cancer.

291. The method of claim 285, wherein a cancer condition in the plurality of cancer conditions is a liquid cancer, a liver cancer, or lung cancer.

292. The method of any one of claims 276-291, wherein the first biological sample and the second biological sample are plasma.

293. The method of any one of claims 276-291, wherein the first biological sample and the second biological sample are different aliquots of the same biological sample from the test subject.

294. The method of any one of claims 276-291, wherein the first biological sample and the second biological sample are the same biological sample.

295. The method of any one of claims 276-291, wherein the first biological sample or the second biological sample comprises blood, whole blood, plasma, serum, urine, cerebrospinal fluid, fecal, saliva, sweat, tears, pleural fluid, pericardial fluid, or peritoneal fluid of the test subject.

296. The method of any one of claims 276-291, wherein the first biological sample or the second biological sample consists of blood, whole blood, plasma, serum, urine, cerebrospinal fluid, fecal, saliva, sweat, tears, pleural fluid, pericardial fluid, or peritoneal fluid of the test subject.

297. The method of any one of claims 276-296, wherein a respective pathogen in the set of pathogens is Epstein-Barr virus (EBV), human cytomegalovirus (HCMV), hepatitis B virus (HBV), hepatitis C virus (HCV), human herpes virus (HHV), human mammary tumor virus (HMTV), human papillomavirus 16 (HPV16), human papillomavirus 18 (HPV18), human papillomavirus 60 (HPV-60), human papillomavirus ZM130 (HPV8-ZM130), human T-cell leukemia virus type 1 (HTLV-1), John Cunningham virus (JCV), molluscum contagiosum virus (MCV), or simian vacuolating virus 40 (SV40).

298. The method of any one of claims 276-296, wherein the set of pathogens is all or a subset of the RefSeq viral genome database.

299. The method of any one of claims 276-296, wherein the set of pathogens comprises any combination of the Epstein-Barr virus (EBV), human cytomegalovirus (HCMV), hepatitis B virus (HBV), hepatitis C virus (HCV), human herpes virus (HHV), human mammary tumor virus (HMTV), human papillomavirus 16 (HPV16), human papillomavirus 18 (HPV18), human papillomavirus 60 (HPV-60), human papillomavirus ZM130 (HPV8-ZM130), human T-cell leukemia virus type 1 (HTLV-1), John Cunningham virus (JCV), molluscum contagiosum virus (MCV), and simian vacuolating virus 40 (SV40).

300. The method of any one of claims 276-296, wherein the set of pathogens comprises any combination of human herpes virus 5 CINCY-TOWNE (HHV5-CINCY-TOWNE) virus, Epstein-Barr B95-8 (EBV-B95-8 virus), molluscum contagiosum virus R17b (MCV-R17b) virus, human papillomavirus 16 (HPV16) virus, human cytomegalovirus AD169 (HCMV-AD169) virus, hepatitis B virus (HBV) virus, hepatitis B virus 18 (HPV18) virus, hepatitis C virus (HCV) virus, human papillomavirus 8-ZM130 (HPV8-ZM130) virus, and John Cunningham virus PLYCG (JCV-PLYCG) virus.

301. The method of any one of claims 276-296, wherein the set of pathogens comprises at least two pathogens.

302. The method of any one of claims 276-296, wherein the set of pathogens comprises at least twenty pathogens.

303. A computer system for screening for a cancer condition in a test subject, the computer system comprising:
one or more processors;
a memory; and
one or more programs, wherein the one or more programs are stored in the memory and are configured to be executed by the one or more processors, the one or more programs including instructions for:
(a) obtaining, in electronic form, a plurality of sequence reads from a first biological sample from the test subject, wherein the first biological sample comprises cell-free nucleic acid from the test subject and potentially cell-free nucleic acid from at least one pathogen in a set of pathogens;
(b) determining, for each respective pathogen in the set of pathogens, a corresponding amount of the plurality of sequence reads that map to a sequence in a pathogen target reference for the respective pathogen, thereby obtaining a set of amounts of sequence reads, each respective amount of sequence reads in the set of amounts of sequence reads for a corresponding pathogen in the set of pathogens; and
(c) using the set of amounts of sequence reads to determine whether the test subject has the cancer condition or a likelihood that the test subject has the cancer condition.

304. A non-transitory computer readable storage medium and one or more computer programs embedded therein for classification, the one or more computer programs comprising instructions which, when executed by a computer system, cause the computer system to perform a method for screening for a cancer condition in a test subject comprising:
(a) obtaining, in electronic form, a plurality of sequence reads from a first biological sample from the test subject, wherein the first biological sample comprises cell-free nucleic acid from the test subject and potentially cell-free nucleic acid from at least one pathogen in a set of pathogens;
(b) determining, for each respective pathogen in the set of pathogens, a corresponding amount of the plurality of sequence reads that map to a sequence in a pathogen target reference for the respective pathogen, thereby obtaining a set of amounts of sequence reads, each respective amount of sequence reads in the set of amounts of sequence reads for a corresponding pathogen in the set of pathogens; and
(c) using the set of amounts of sequence reads to determine whether the test subject has the cancer condition or a likelihood that the test subject has the cancer condition.
